(19) United States
(12) Patent Application Publication
(10) Pub. No.: US 2019/0256592 A1
(43) Pub. Date: Aug. 22, 2019

(54) BINDING AGENTS FOR USE IN THERAPY

(71) Applicant: GENAGON THERAPEUTICS AB, Solna (SE)

(72) Inventors: Olof Andries BLOKZIJL, Stockholm (SE); Johan Erik Simon FREDRIKSSON, Bromma (SE)

(21) Appl. No.: 16/333,552

(22) PCT Filed: Sep. 15, 2017
(86) PCT No.: PCT/EP2017/073338

§ 371 Date: Mar. 14, 2019

(30) Foreign Application Priority Data
Sep. 16, 2016 (GB) 1615844.6

(51) Int. Cl. C07K 16/28; A61P 1/00; A61P 3/08; A61P 19/02; A61P 37/00

(52) U.S. Cl. CPC C07K 16/28; A61P 1/00; A61P 3/08; A61K 2039/505; A61P 37/00; C07K 2317/34; A61P 19/02

(57) ABSTRACT

The present invention providesabinding agent capable of binding to the extracellular domain of CLPTM1 for use in the treatment or prevention of a condition which is responsive to, or benefits from, (i) immunosuppression, (ii) the reduction or reversal of one or more pro-inflammatory cytokines or the induction of an anti-inflammatory cytokine), (iii) an increase in insulin sensitivity, or (iv) therapy with GDF15 and/or TGF-β and/or IFNβ, wherein said binding agent has an EC 50 value of 1 μg/ml or less when determined by measuring binding to membrane-permeabilised O-876 cells expressing native CLPTM1 by flow cytometry, and wherein said binding agent is not a natural ligand for CLPTM.

The present invention relates to binding agents for Cleft Lip and Palate Transmembrane protein 1 (CLPTM1), and in particular high affinity binding agents, which are capable of binding to CLPTM1, and their use in therapy, including particularly as anti-inflammatory and/or immunosuppressive agents, that is in the treatment or prevention of inflammatory conditions and/or conditions which respond to, or benefit from, immunosuppression, as well as metabolic disorders, including particularly insulin resistance and disorders or conditions associated therewith, and/or other conditions responsive to therapy with GDF15 and/or TGF-β and/or interferon β (IFNβ). In particular, as reported herein, the binding agents are able to mediate immunosuppressive effects (or put more generally, mediate immunosuppression) through (or alternatively expressed, by binding to) the CLPTM1 receptor.

CLPTM1 is a transmembrane protein with a ˜350 amino acid extracellular domain (the amino acid sequence of the ECD of human CLPTM1 is shown in SEQ ID NO:2 and has 353 amino acids (representing amino acids 2-354 of SEQ ID NO:1)). CLPTM1 has an unusual expression pattern and is expressed, for instance, in the cells of the immune system. In the work leading to the present invention, we have discovered that CLPTM1 is expressed in Natural Killer cells (NK-cells) and macrophages, and we have further shown that it may be expressed by other cells of the immune system, such as various classes of lymphocytes, particularly various classes of T-lymphocytes; particular sub-sets of CD4+, CD8+ T-cells may express CLPTM1, as may particular subsets of CD3 CD45+ non-T-cells. In particular we have demonstrated cell surface expression. Thus, whilst CLPTM1 is expressed by a wide range of different cell types, as described in greater detail below, in many cases this expression is intracellular and it is only in certain cell types and in response to certain physiological stimuli that the CLPTM1 is expressed at the cell surface.

We have previously reported that CLPTM1 binds to Growth and Differentiation Factor 15 (GDF15) (also known as MIC-1) and have proposed that CLPTM1 may be a receptor for GDF15. We have also identified that GDF15 is capable of binding to the pyroglutaminated RFamide Peptide Receptor (QRFPR). Binding agents for these receptors, specifically binding agents which inhibit the binding and/or effect of GDF15 at these receptors (i.e. antagonistic binding agents, or more particularly agents which are antagonistic with respect to the effect of GDF15 at the receptors) and polypeptides derived from these receptors and in their use in various therapies are the subject of commonly-owned patent application WO 2017/013188, the disclosure of which is hereby incorporated by reference in its entirety.

GDF15 is a member of the TGF-β superfamily, and has a relatively low (24%) sequence homology with other members of the TGF-β superfamily. Elevated levels of GDF15 have been implicated in cancer, anorexia nervosa, osteoporosis, kidney disorders, pulmonary arterial hypertension, and cardiovascular disease, and also in cachexia and more generally in loss or suppression of appetite. GDF15 is a marker for mortality by any cause. The therapies proposed in WO 2017/013188 are based on inhibiting the effect of GDF15, and thus for treating or preventing a condition associated with elevated or unwanted levels of GDF15, including the conditions listed above, and with respect to the receptor CLPTM1, particularly on reducing the immunosuppressive effects of GDF15, for example in the treatment of cancer.

However, we now believe that the interactions of various ligands at the CLPTM1 receptor, and the role played by the receptor, may be more complex, and that in fact, agents which bind to CLPTM1 may have an immunosuppressive and/or anti-inflammatory effect. In the work leading to the present invention, we have identified that the level of CLPTM1 expressed at the cell surface in immune cells is increased in response to an inflammatory stimulus. As described in more detail in the Examples below, stimulation of both macrophages and NK-cells with LPS and CD3/CD28 beads respectively results in elevated levels of CLPTM1 at the cell surface. This inflammatory stimulus is thought to drive CLPTM1, which is normally located in the endoplasmic reticulum (ER), to localise to the cell surface, and immune cells stimulated in this way may represent a model for the immune system that has been primed or activated in response to an inflammatory stimulus. It is therefore believed that immune cells stimulated in this way in vitro may be representative of immune cells in conditions such as autoimmune conditions or cancers.

We now propose that agents which bind to the extracellular domain of CLPTM1 with a high affinity, and/or which agents which bind to CLPTM1 and are capable of down-regulating the STING receptor (Stimulator of Interferon Genes), also known as TMEM173, may exert anti-inflammatory and/or immunosuppressive effects, as described in more detail below. STING is the master interferon regulator in humans and the effects of binding agents on this protein are discussed further below,

Surprisingly, we have observed that certain antibodies raised against CLPTM1 can alter the cytokine secretome of a number of different immune cells. Specifically, and as described in greater detail in the Examples below, contacting immune cells which have been stimulated with LPS with an antibody raised against CLPTM1 was found to result in alterations in the cytokine secretion profile, with anti-inflammatory cytokines increased, and pro-inflammatory cytokines decreased.

These data suggest that CLPTM1 may represent a potential therapeutic target for the treatment of a number of inflammatory conditions. Put another way, CLPTM1 may be targeted to achieve immunosuppression.

We have further observed that there is a correlation between the affinity of a binding agent for CLPTM1 and the ability of a binding agent to cause the effects described above. Specifically, we have identified that whilst binding agents having a higher affinity for CLPTM1 are capable of causing the above effects, antibodies with lower affinity for CLPTM1 do not show these effects, or they are seen to a much lesser extent. High-affinity binding agents may accordingly be considered to be effective agents for causing the reduction in inflammation described above.

Based on these observations, we propose that binding agents for CLPTM1 may be of benefit in the treatment of a number of inflammatory diseases or disorders and autoimmune conditions, or indeed any condition where immunosuppression may be of benefit.

Such proposals are also supported by further work. Thus, we have also discovered that Transforming Growth Factor-beta proteins (TGF-β), including TGF-β1, 2 and 3 are also capable of binding to CLPTM1. TGF-β cytokines belong to the TGF-β superfamily, and TGF-β3 is the closest relative to GDF15. TGF-β cytokines are involved in many different aspects of cell differentiation, embryogenesis and development, and are thought to be involved in regulating cellular adhesion and extracellular matrix formation during palate development. Knockout of TGF-β3 is associated with cleft lip formation in mice and is only partially rescued by TGF-β1 knock-in, indicating that this protein has specific biological roles. TGF-β, including particularly TGF-β3, also have a known immunosuppressive role (Okamura T et. al Nat Commun (2015), and elevated levels of TGF-β3 have been found to be associated a number of different cancers, including breast, pancreatic and prostate cancers, and mesothelioma and sarcoma (The cancer genome atlas, RNA-seq database as accessed through UCSC) (https://genome-cancer.soe.ucsc.edu).

Without wishing to be bound by theory, given the degree of homology between GDF15 and TGF-β, and their partially overlapping range of biological functions, it is possible that both GDF15 and TGF-β effect the same or similar biological roles, possibly through the same receptor, CLPTM1.

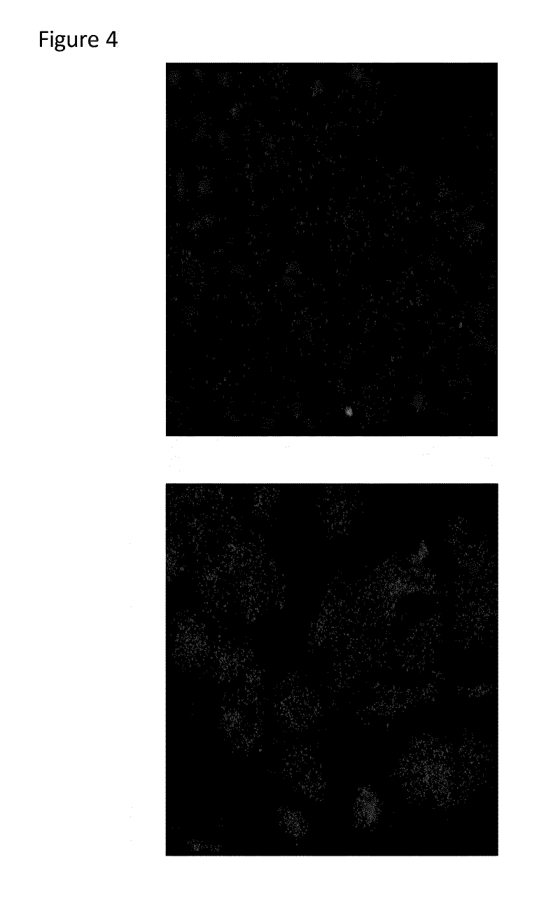

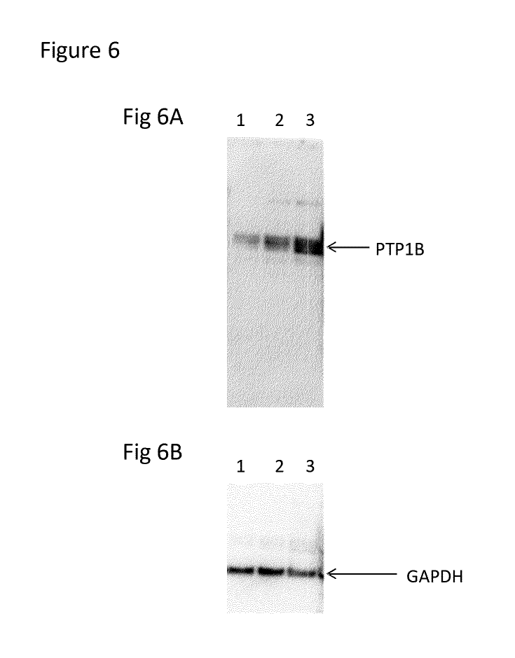

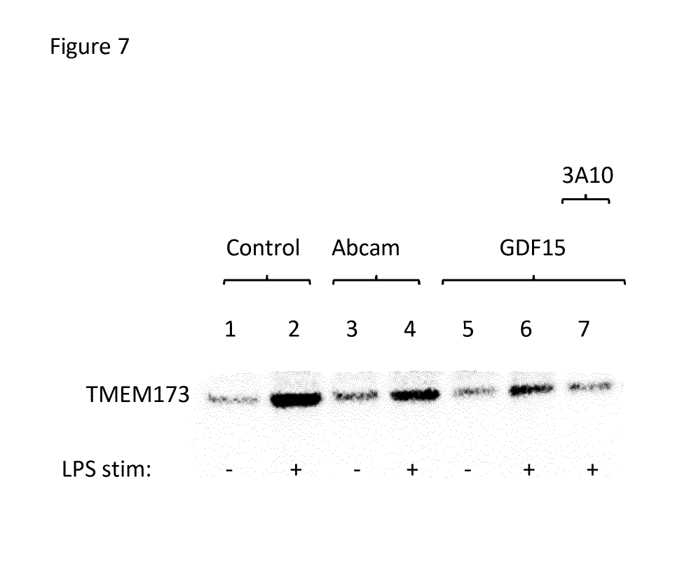

We have found that GDF15 or TGF-β3 can alter the cytokine secretome of a variety of different immune cells, increasing secretion of anti-inflammatory cytokines and decreasing secretion of pro-inflammatory cytokines, an effect similar to that seen with antibodies which bind to CLPTM1. We have also demonstrated that GDF15 and TGF-β3 can reduce the activity of TMEM173/STING, and can also induce degradation of protein-tyrosine phosphatase 1B (PTP1B), a phosphatase known to be implicated in several inflammatory diseases (and in regulating insulin sensitivity—see further below). Specifically, we have found that the addition of GDF15 to immune cells expressing CLPTM1 results in formation of the CLPTM1-TMEM173/STING complex (FIG. 4), accumulation of TMEM173/STING-Rab11 double positive endosomes (FIG. 7), and degradation of PTP1B (FIG. 6). We further report that the activity of TMEM173/STING can be reduced using an antibody binding to CLPTM1 (Example 13).

Without wishing to be bound by theory, it is thought that the changes in the cytokine secretome seen for TFGβ and GDF15 occur through the changes in TMEM173/STING and PTP1B described above, and it is proposed that anti-CLPTM1 antibodies may also exert their effects via these proteins (specifically through down-regulation of one or both of these proteins).

The STING receptor plays an important role in innate immunity, and is capable of detecting cytosolic bacterial and viral DNA to promote the production of type I interferon (including IFN-alpha and IFN-beta) in response to intracellular pathogens. TMEM173/STING mediates the type I interferon immune response by functioning as both a direct DNA sensor and a signalling adaptor protein. It has also been shown to activate downstream transcription factors STAT6 and IRF3 through TBK1, which are responsible for antiviral response and innate immune response against intracellular pathogens. Phosphorylated IRF3 and STAT6 each form homodimers which enter the nucleus and stimulate expression of genes involved in immune responses, such as CCL2 and CCL20 (Chen H et al. 2011. Cell. 147, 436-46). STING activation also utilises nuclear factor KB (NF-κB) to exert its effects. Interleukin-6 (IL-6), IL-1β, and tumour necrosis factor alpha (TNF-α) expression is also known to require the IRF3 and NF-κB pathways (Paludan S R and Bowie A G. 2013. Immunity 23, 870-880). The protein has also been shown to play a role in apoptotic signalling by associating with the type II major histocompatibility complex (Type II MHC) (Jin et al. 2008. Mol Cell Biol 28, 5014-5026).

PTP1B is known to dephosphorylate phosphotyrosine residues of the activated insulin receptor kinase resulting in reduced insulin sensitivity (Goldstein et al J Biol Chem 2000, February 11; 275(6):4283-9). Knockout studies of PTP1B in mice have observed enhanced insulin sensitivity (Elchebly M. et al. 1999. Science. 283, 1544-8; Klaman L D. et al. 2000. Mol. Cell. Biol. 20, 5479-89). Other recent studies have also shown that PTP1B inhibitors can enhance insulin sensitivity, as described for example in WO 2013158970, US2016/012099, WO 2015198199 and US2016/0015784. Down-regulation of PTP1B activity is therefore thought to represent a possible mechanism for enhancing insulin sensitivity in a subject with reduced insulin sensitivity (or a subject with insulin resistance), in particular patients with type 2 diabetes. We have found that binding of ligands such as GDF15 and TFGβ to CLPTM1 results in degradation of PTP1B, and binding agents for CLPTM1 which result in PTP1B down-regulation may also be suitable for this purpose.

PTP1B is also known to be associated with a number of inflammatory diseases and may therefore provide a key link between metabolism and inflammation. PTP1B is known to regulate the VEGF pathway, dephosphorylating and inactivating the VEGF receptor VEGFR2 (Lanahan et al. 2010. Dev Cell 18, 713-724). VEGFR2 is normally expressed in the endothelium, but has been found to be expressed in certain tumour-associated macrophages (TAMS), which secrete placental growth factor (PIGF) in an autocrine loop as pair of their cytokine secretion profile. Overexpression of VEGF in brown and white adipose tissue has also been found to result in increased adipose vascularisation and M2 anti-inflammatory macrophages in adipose tissue, as well as reduced insulin resistance (Elias et al. 2012. Diabetes 61, 1801-1813). Together, these studies suggest that reduced PTP1B activity may result in increased PIGF secretion via increased VEGFR2 activity. CLPTM1 may therefore represent an attractive target for treating any such disease.

As noted above, and as described in greater detail in the Examples below, we show that GDF15 or TGF-β3, possibly acting through CLPTM1, may change the cytokine secretome of a number of different immune cells, in particular, in a reduction of certain pro-inflammatory cytokines, and an increase in certain anti-inflammatory cytokines, which will result in reduced inflammation. Notably, PIGF expression was found to be elevated upon antibody-induced activation of CLPTM1, which suggests that at least a component of the change in the cytokine expression profile shown by immune cells contacted with a binding agent of the present invention may be due to degradation of PTP1B.

Although the signalling pathways underlying TMEM173/STING and PTP1B and the involvement of CLPTM1 have recently been elucidated (Elisa Reimer, PhD Thesis, Identification and characterization of proteins involved in proper functioning of UNC93B-Toll-like receptor complexes, Technical University Braunschweig 2014) the connection between these pathways and the CLPTM1 circulating ligands had previously been unknown, and the link between GDF15 or TGF-β proteins and lowering the activities of TMEM173/STING or PTP1B has until now not been known.

The present application is therefore directed towards binding agents for CLPTM1, and particularly those which have been found to mimic the effects of GDF15 and/or TGF-β, particularly the effects of GDF15 and/or TGF-β in altering the cytokine secretome of immune cells, and/or in down-regulating the activity of STING.

Accordingly, in a first aspect, the present invention provides a binding agent capable of binding to the extracellular domain of CLPTM1 for use in the treatment or prevention of a condition which is responsive to, or benefits from, (i) immunosuppression, (ii) the reduction or reversal of one or more pro-inflammatory cytokines or the induction of an anti-inflammatory cytokine), (iii) an increase in insulin sensitivity, or (iv) therapy with GDF15 and/or TGF-β and/or IFNβ, wherein said binding agent has an EC₅₀ value of 1 μg/ml or less when determined by measuring binding to membrane-permeabilised 0-876 cells expressing native CLPTM1 by flow cytometry, and wherein said binding agent is not a natural ligand for CLPTM1.

In another aspect, the present invention provides a method of treating or preventing a condition which is responsive to, or benefits from, (i) immunosuppression, (ii) the reduction or reversal of one or more pro-inflammatory cytokines or the induction of an anti-inflammatory cytokine), (iii) an increase in insulin sensitivity, or (iv) therapy with GDF15 and/or TGF-β and/or IFNβ, said method comprising administering to a subject in need thereof an effective amount of a binding agent for CLPTM1, wherein said binding agent has an EC₅₀ value of 1 μg/ml or less when determined by measuring binding to membrane-permeabilised 0-876 cells expressing native CLPTM1 by flow cytometry, and wherein said binding agent is not a natural ligand for CLPTM1.

In yet another aspect, the present invention also provides the use of a binding agent for CLPTM1 in the manufacture of a medicament for use in the treatment or prevention of a condition which is responsive to, or benefits from, (i) immunosuppression, (ii) the reduction or reversal of one or more pro-inflammatory cytokines or the induction of an anti-inflammatory cytokine), (iii) an increase in insulin sensitivity, or (iv) therapy with GDF15 and/or TGF-β and/or IFNβ, wherein said binding agent has an EC₅₀ value of 1 μg/ml or less when determined by measuring binding to membrane-permeabilised 0-876 cells expressing native CLPTM1 by flow cytometry, and wherein said binding agent is not a natural ligand for CLPTM1.

As noted above, we have observed that the changes in the cytokine secretome of immune cells seen on addition of a high affinity binding agent for CLPTM1 are similar to those seen on the addition of GDF15, and that said addition also results in similar reductions in the levels of IFN-α to TGF-β3. We have also observed that the activity of TMEM173/STING can be lowered using such a binding agent. Without wishing to be bound by theory, it is thought that the changes in the cytokine secretome may occur through the changes in TMEM173/STING and PTP1B described herein, which are otherwise associated with TGFβ and GDF15.

In particular, based on our in vitro studies, it is proposed that in one embodiment the binding agent binding at the CLPTM1 receptor mimics at least one effect of GDF15 and/or TGF-β (e.g. TGF-β1, 2 and/or 3) on cells expressing CLPTM1). Without wishing to be bound by theory, this may be an effect of GDF15 and/or TGF-β (e.g. TGF-β1, 2 and/or 3) binding at the CLPTM1 receptor. Alternatively, this may be an effect of GDF15 and/or TGF-β (e.g. TGF-β1, 2 and/or 3) on CLPTM1-expressing cells which is achieved by another mechanism.

In other particular embodiments the binding agent is capable of down-regulating (or inhibiting, or reducing the activity of, etc.) TMEM173/STING and/or PTP1B (e.g. it is capable of resulting in, or causing or inducing, the degradation of PTP1B), and/or capable of promoting or inducing an anti-inflammatory cytokine profile (or cytokine environment) or reducing or reversing a pro-inflammatory cytokine profile (or cytokine environment). It will be seen therefore that the binding agent for use according to the invention is immunosuppressive. That is, it is capable of causing, promoting or inducing an immunosuppressive effect when administered to a subject. Alternatively expressed, the binding agent is capable of exerting an immunosuppressive effect, e.g. on immune cells, and/or it is capable of exerting an anti-inflammatory effect, e.g. on immune cells.

The property of down-regulating TMEM173/STING is of particular interest. Accordingly, in a further aspect, the present invention provides a binding agent capable of binding to the extracellular domain of CLPTM1 for use in the treatment or prevention of a condition which is responsive to, or benefits from, (i) immunosuppression, (ii) the reduction or reversal of one or more pro-inflammatory cytokines or the induction of an anti-inflammatory cytokine), (iii) an increase in insulin sensitivity, or (iv) therapy with GDF15 and/or TGF-β and/or IFNβ, wherein said binding agent is capable of down-regulating TMEM173/STING and wherein said binding agent is not a natural ligand for CLPTM1.

In another aspect, the present invention provides a method of treating or preventing a condition which is responsive to, or benefits from, (i) immunosuppression, (ii) the reduction or reversal of one or more pro-inflammatory cytokines or the induction of an anti-inflammatory cytokine), (iii) an increase in insulin sensitivity, or (iv) therapy with GDF15 and/or TGF-β and/or IFNβ, said method comprising administering to a subject in need thereof an effective amount of a binding agent for CLPTM1, wherein said binding agent is capable of down-regulating TMEM173/STING, and wherein said binding agent is not a natural ligand for CLPTM1.

In yet another aspect, the present invention also provides the use of a binding agent for CLPTM1 in the manufacture of a medicament for use in the treatment or prevention of a condition which is responsive to, or benefits from, (i) immunosuppression, (ii) the reduction or reversal of one or more pro-inflammatory cytokines or the induction of an anti-inflammatory cytokine), (iii) an increase in insulin sensitivity, or (iv) therapy with GDF15 and/or TGF-β and/or IFNβ, wherein said binding agent is capable of down-regulating TMEM173/STING, and wherein said binding agent is not a natural ligand for CLPTM1.

Down-regulation of TMEM173/STING according to the present invention encompasses any effect of reducing the activity and/or expression of TMEM173/STING. In particular, the activity of TMEM173/STING is reduced. This may be by any effect on the TMEM173/STING protein, and may include a reduction in the amount of TMEM173/STING protein, e.g. by degradation of the protein, or any effect in reducing the activity of TMEM173/STING, howsoever achieved, e.g. by inhibiting the activity of the protein. In a particular embodiment, the down-regulation may be down-regulation of the amount of TMEM173/STING protein. This may lead to reduced, or down-regulated activity of TMEM173/STING.

Metabolic disorders, including particularly metabolic disorders associated with insulin resistance, represent conditions of particular interest according to the present invention.

In a still further aspect, the invention therefore also provides a binding agent capable of binding to CLPTM1 for use in the treatment or prevention of a metabolic disorder, including insulin resistance or a condition associated therewith.

According to this aspect also provided is use of a binding agent capable of binding to CLPTM1 for the manufacture of a medicament for use in the treatment or prevention of a metabolic disorder, including insulin resistance or a condition associated therewith.

Further also provided is a method of treating or preventing a metabolic disorder in a subject, including insulin resistance or a condition associated therewith, said method comprising administering a binding agent capable of binding to CLPTM1 (particularly an effective amount thereof) to a subject in need thereof.

Further aspects of the invention also provide a product comprising a binding agent capable of binding to CLPTM1 and interferon-G3 as a combined preparation for separate, simultaneous or sequential use in the treatment of an autoimmune or inflammatory condition, including multiple sclerosis; and

a product comprising a binding agent capable of binding to CLPTM1 and a TNF-blocker as a combined preparation for separate, simultaneous or sequential use in the treatment of an autoimmune or inflammatory condition.

The binding agent for use according to such further aspects of the invention may be any binding agent as defined and described herein. It is believed that one of the roles of CLPTM1 may be to down-regulate signalling by certain members of the TGFβ superfamily, by inhibiting TGFβ receptor activation. More particularly, it is believed that this may be achieved by the action (or effect) of CLPTM1 down-regulating TGFBR1 activity. Without wishing to be bound by theory, it is proposed that CLPTM1 may act a negative regulator, or brake, to reduce or down-regulate TGF-β signalling (e.g. via TGFBR1). Thus, CLPTM1 may act to promote, or up-regulate, an immune response in the body, by down-regulating or reducing immunosuppressive effects. In other words CLPTM1 (or more specifically signalling through CLTPM1) may have an effect of reducing immune dampening, or reducing immunosuppression. Elevated levels of CLPTM1 at the cell surface, which as noted above may occur in certain conditions, e.g. in inflammatory conditions, may therefore lead to a reduction in the level of TGFβ signalling in a cell. Without wishing to be bound by theory, binding agents (notably binding agents with sufficiently high affinity for CLPTM1) may be able to bind to CLPTM1 and prevent such inhibition of TGFβ signalling, thereby allowing increased TGFβ signalling to take place. Both GDF15 and TGFβ, which may be natural ligands for CLPTM1 as described in greater detail below, are believed also to be ligands to TGFBR1, and thus signalling by either of these proteins might be expected to increase once CLPTM1 inhibition of TGFBR1 is relieved. This may provide a possible mechanism for how the binding agents of the present invention are able to replicate or mimic the downstream effects of GDF15 and TGFβ.

A natural ligand for CLPTM1 is any ligand (i.e. any compound or molecule or moiety etc.) which binds to CLPTM1 when present in a human or animal body. The term thus includes any endogenous ligand of CLPTM1. As noted above, we believe that GDF15 and TGF-β may be endogenous ligands for CLPTM1. Thus, in particular, the binding agent is not GDF15 and/or TGF-β, whether human or from any species. In another embodiment the binding agent is not GDF15 and/or TGF-β1, 2 and/or 3. The term “natural ligand” also includes other native, natural, or wild-type ligands (in the sense of molecules, or compounds etc.), other than antibodies, which may bind to CLPTM1. (Thus the term “natural ligand” does not include an antibody, and more generally the term “ligand” as used herein does not include an antibody or antibody-like molecule). Accordingly, for the avoidance of doubt an autoimmune antibody, or indeed any other antibody, is not included under the term “natural ligand”, and is accordingly not excluded from the scope of the invention under the disclaimer that the binding agent is not a natural ligand for CLPTM1.

In certain other embodiments the binding agent for use according to the invention is not an interferon and in particular it is not IFNβ.

As used herein, a pro-inflammatory cytokine profile means that a set of cytokines (which term is used broadly herein to include all cytokines and chemokines) is produced and secreted (and may be detected in a subject) which includes an increase in cytokines associated with a pro-inflammatory effect. Thus, the levels of one or more cytokines which are predominantly associated with a pro-inflammatory effect are increased. The overall cytokine environment is thus, or tends towards being, pro-inflammatory. Conversely, an anti-inflammatory cytokine profile means that a set of cytokines (which term is used broadly herein to include all cytokines and chemokines) is produced and secreted (and may be detected in a subject) which includes an increase in cytokines associated with an anti-inflammatory effect. Thus, the levels of one or more cytokines which are predominantly associated with an anti-inflammatory effect are increased. The overall cytokine environment is thus, or tends towards being, anti-inflammatory.

More generally, the binding agent may be capable of promoting or inducing an anti-inflammatory secretome (or environment) or reducing or reversing a pro-inflammatory secretome (or environment), and/or the condition may be a condition which is responsive to, or benefits from, a reduction or reversal of a pro-inflammatory secretome (or environment) or the induction of an anti-inflammatory secretome (or environment). A secretome (or environment) may comprise, in addition to cytokines, other secreted proteins or components, for example binding proteins or receptors for the cytokines, enzymes, signalling molecules etc. Thus, a secretome may comprise other immune effector or signalling molecules, pathway components etc. which are involved in how the cytokines work, and which may therefore modulate (increase or decrease) their function.

In certain embodiments, the binding agent of the invention may be capable of decreasing the level of TNFα. In particular, the binding agent when pre-incubated at 1 μg/ml for 16 hours with CD14+ cells induces a 3-fold decrease in the level of secreted TNFα when the CD14+ cells are stimulated with 1 ng/ml LPS for 4 hours.

In another embodiment, the binding agent may be capable of decreasing the level of IL12. In particular, the binding agent when pre-incubated with CD14+ cells at 1 μg/ml for 16 hours induces a 2-fold decrease in the level of secreted IL12 when the CD14+ cells are stimulated with 1 ng/ml LPS for 4 hours.

The present invention may thus be used to normalise, or to counteract, a dysregulated cytokine secretome. It may thus be used in the treatment or prevention of conditions associated with unwanted or elevated cytokine activity, e.g. hypercytokinemia, (also known as a cytokine storm or surge). Hypercytokinemia is a potentially fatal immune reaction consisting of a positive feedback loop between cytokines and white blood cells, resulting in severe and uncontrolled release of cytokines, with levels of various different cytokines being highly elevated, and may be a result of various diseases and conditions, including a disease or condition as described herein, and in particular conditions such as sepsis, malaria, graft versus host disease (GVHD) etc. Hypercytokinemia can result in, or increase the risk of, multiple organ dysfunction syndrome (MODS), which is also known as multiple organ failure (MOF) or multisystem organ failure (MSOF). In particular, hypercytokinemia may be the result of a very severe infection, e.g. in the case of sepsis and septicaemia, where it is thought that overproduction of cytokines contributes to symptoms and complications associated with these conditions, e.g. multiple organ dysfunction syndrome (i.e. multiple organ failure).

Indeed uncontrolled or excessive cytokine release (e.g. hypercytokinemia) from immune cells in blood, such as monocytes, is associated with a variety of disorders. For instance, it is thought that the excess release of pro-inflammatory cytokines is the primary driving force of disease and death caused by Plasmodium falciparum infections, i.e. malaria. Other infectious diseases, including bacterial, fungal and some viral infections (e.g. influenza, such as swine influenza, avian influenza), may result in excessive pro-inflammatory cytokine release in blood. Such infections may be local or systemic. In addition, allogenic tissue transplants can result in graft versus host disease, in which immune cells produce excess levels of cytokines such as TNFα.

In particular embodiments of the above aspects of the invention (and indeed any other aspects, as indicated below), the binding agent is used, or for use, in the treatment or prevention of an inflammatory condition, a condition which is responsive to, or which benefits from, immunosuppression (which condition may include aberrant (e.g. unwanted or increased) immune activity), a metabolic disorder, particularly a metabolic disorder associated with insulin resistance (or reduced sensitivity to insulin), or a condition involving damage to the heart, whether caused by injury or disease.

Thus, the binding agent may be used, or may be for use, as an immunosuppressive agent, an anti-inflammatory agent, an anti-insulin resistance agent and/or a cardioprotective agent. In other words, the agent may be used to achieve, or promote, immunosuppression, or cardioprotection, or an anti-inflammatory effect, or an effect of increasing insulin sensitivity (or reducing insulin resistance).

In other particular embodiments, or indeed more generally in certain aspects of the invention, the binding agent may be used to treat an autoimmune condition, a haematological disorder (e.g. a haematopoietic cancer), an infectious disease or condition, particularly an infection with an intracellular pathogen in macrophages, an allergy or allergic reaction, organ or tissue rejection following transplant, insulin resistance or a condition associated therewith, damage to the heart associated with myocardial infarction (MI) or any acute coronary syndrome or any ischaemic condition, NASH, including NAFLD, preeclampsia, lung disorders, including particularly emphysema or COPD, local internal inflammation, scars, fibrosis or radiation-induced damage.

Particularly in the context of treating or preventing an inflammatory condition or for immunosuppression, the invention may be used to achieve a selective immune modulation.

The various therapies proposed above, and various rationales therefor, will now be discussed in more detail below.

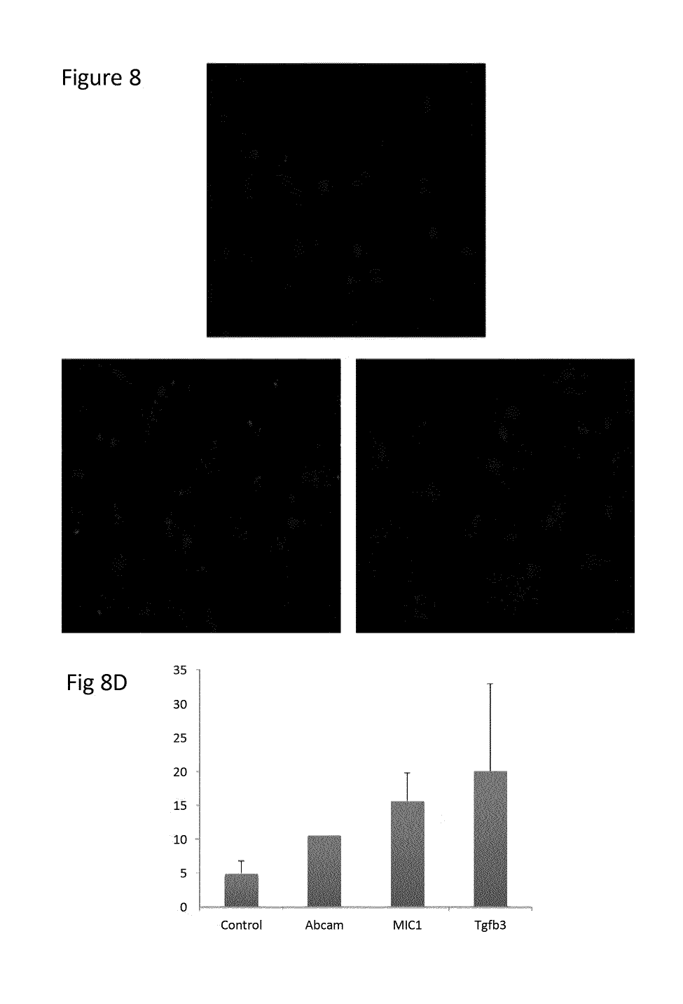

The binding agents of the present invention may act to reduce the level of expression of CLPTM1 on the surface of a cell. Example 7 (FIG. 8D) indicates that an anti-CLPTM1 antibody may cause the receptor to become internalised in endosomes. It is believed that certain binding agents for CLPTM1 may result in the efficient internalisation of CLPTM1, (which may be determined by measuring the degree of uptake of fluorescently-labelled antibodies for CLPTM1) However, whilst this may be a beneficial property of certain binding agents of the invention, it is not an essential requirement.

The binding agents of the present invention may in some embodiments be used to treat any condition that is associated with aberrant (i.e. elevated or unwanted) expression or activity of CLPTM1. In particular, any condition associated with increased or elevated expression of CLPTM1 on the extracellular surface of a cell, particularly an immune cell and especially an immune effector cell such as a NK cell, a macrophage or a T-cell, may be treated or prevented using a binding agent of the present invention. Alternatively expressed, the condition may be responsive to, or may benefit from, a reduction in CLPTM1 expression or activity at a cell surface. This may include various inflammatory conditions, and in particular autoimmune conditions (including multiple sclerosis (MS), Systemic Lupus Erythematosus (SLE) and juvenile arthritis, as discussed further below).

It is noted, however, that certain non-haematopoietic cancers are known to have elevated levels of CLPTM1, in particular at their cell surface, and such conditions may be considered as falling outside the scope of the present invention. Accordingly, in certain embodiments, a condition associated with aberrant expression or activity of CLPTM1 does not include non-haematopoietic cancers. Put another way, in certain embodiments, such a condition is not a non-haematopoietic cancer.

As described above, the work leading to the present invention resulted in the discovery that CLPTM1 may be found at the cell surface and that surprisingly, binding agents for CLPTM1 were found to induce similar effects to those seen when cells expressing CLPTM1 were contacted with GDF15 or TGFβ. In particular, these proteins ware found to activate intracellular signalling pathways, culminating in the reduction in the activity and/or level of TMEM173/STING and PTP1B. Surprisingly, an antibody for CLPTM1 (i.e. a binding agent as described herein) was found to mimic this effect, and suggests that such binding agents could be used to inhibit and modulate these downstream signalling pathways. Such binding agents may therefore be useful in the treatment or prevention of any condition (i.e. any disease or disorder) which is known to be associated with such pathways, particularly conditions associated with increased or elevated levels or activity of TMEM173/STING or PTP1B. Accordingly, the binding agents of the present invention may be for use, or used, in the treatment of a condition associated with elevated levels or activity TMEM173/STING or PTP1B. This category will also include autoimmune conditions, including e.g. MS, as well as infectious diseases.

In one aspect of the present invention, CLPTM1 binding agents may reduce the response of certain types of immune cell to an inflammatory stimulus. In this aspect of the invention, binding agents capable of binding to CLPTM1 may be capable of treating or preventing a condition associated with unwanted or elevated immune activity, or in other words, may result in a reduction in immune activity (i.e. an immunosuppressive effect).

A reduction in immune activity may be a reduction in the activation or efficacy of the immune system, i.e. immunosuppression. Such a reduction may be specific, i.e. targeted only at hyperactive components of the immune system, or may be systemic, i.e. targeted broadly at a number of different components of the immune system. A reduction in immune activity may also encompass decreasing the rate of growth, division, proliferation and/or maturation of immune cells, including immature immune cells.

A reduction in immune activity may be achieved, for example, by altering the expression of one or more cytokines by cells of the immune system. As noted above and described in more detail in the Examples, CLPTM1 binding agents can alter the secretion of particular cytokines by immune cells. As described below in greater detail, contacting immune cells with a binding agent of the present invention has been found to alter the expression profiles of a number of (10 or more) different cytokines and associated proteins. Thus, up-regulation of anti-inflammatory cytokines and down-regulation of pro-inflammatory cytokines has been observed in response to a binding agent of the present invention. The expression of a number of different cytokines may therefore be altered in this way, using a binding agent of the present invention.

Accordingly, CLPTM1 represents an attractive new treatment target, since advantageously a single active agent may (i.e. a binding agent according to the present invention) be used to alter, or regulate, the production of a number of different cytokines. Thus conditions that are associated with an elevated level of a pro-inflammatory cytokine may be treated using the binding agents of the present invention, by reducing the expression or secretion of the pro-inflammatory cytokine through binding to CLPTM1. Pro-inflammatory cytokines are cytokines that are important in cell signalling and promote inflammation, and work with other components of the immune system such as neutrophils and leukocytes to effect an inflammatory response. Pro-inflammatory cytokines include chemokines (e.g. CXCL9, CXCL10 and CXC11), IL-1 IL-1β, IL-2, IL-6, IL-8, IL-12, IL-13, TNF-alpha, IFNα, IFNβ, and IFNγ. Anti-inflammatory cytokines include GDF15, PIGF, FGF19, TGF-β1, MIC-A and IL10. Similarly, any condition that may be treated or prevented by an increase in the level of an anti-inflammatory cytokine in response to binding of a CLPTM1 binding agent to CLPTM1 is included in the scope of the invention.

The proposed immunosuppressive effects of the binding agents of the present invention may be of particular utility in the treatment or prevention of inflammatory conditions, including autoimmune conditions (autoimmune diseases).

Inflammation is part of a biological response to certain harmful stimuli, such as pathogens, damaged cells or irritants, and is a mechanism of innate immunity that serves to eliminate the initial cause of cell injury, clear out necrotic cells and damaged tissue, and initiate tissue repair.

Acute inflammation is an initial response to a harmful stimulus and is characterised by increased movement of plasma and leukocytes (particularly granulocytes) into the injured tissue. Acute inflammation is initiated by immune cells resident in tissue, typically including macrophages, dendritic cells, histiocytes, Kupffer cells and mast cells. Activation of the immune cells results in release of inflammatory mediators responsible for the clinical signs of inflammation, including certain cytokines. Chronic inflammation leads to a progressive shift in the type of cells present at the site of inflammation (such as mononuclear cells), and is characterised by simultaneous destruction and healing of tissue.

An inflammatory response may typically be initiated by activation of a pattern recognition receptor, which may detect microbial or damage-associated molecular patterns. Examples of pattern recognition receptors include membrane-bound PRRs, such as Toll-like receptors (TLR) (which as discussed elsewhere detect intracellular DNA), C-type lectin receptors (CLR) (which detect specific sugars on the surface of bacteria), and cytoplasmic PRRs, such as Nucleotide-binding Oligomerisation Domain (NOD) (which detect peptidoglycan). These proteins typically transduce signals in the NF-κB and MAP kinase pathways to effect an inflammatory response. Activated immune cells in tissue release pro-inflammatory cytokines in response to an inflammatory stimulus. For example, macrophages in tissue release cytokines such as IL-1 and TNF-α, whereas T-cells and NK cells release IL-8.

The invention includes the treatment or prevention of any inflammatory condition (which term includes any inflammatory disease or any condition having an inflammatory component), including conditions associated with acute or chronic inflammation. “Chronic inflammation” generally means an inflammation (e.g. an inflammatory condition) that is of persistent or prolonged duration in the body of a subject. Generally speaking this means an inflammatory response or condition of duration of 20, 25 or 30 days or more or 1 month or more, more particular of at least 2 or 3 months. Chronic inflammation leads to a progressive shift in the type of cells present at the site of inflammation. Chronic inflammation may occur as a result of persistent or prolonged injury or infection, prolonged exposure to toxic substances or by autoimmune responses or conditions. Chronic inflammation may be a factor in the development of a number of diseases or disorders, including particularly degenerative diseases, or diseases or conditions associated with loss of youthful function or ageing.

Included under inflammatory conditions are conditions associated with systemic inflammation, that is inflammation which is not confined to a particular tissue or site or location in the body. The inflammation may be generalised throughout the body. Systemic inflammation typically involves the endothelium and other organ systems.

An inflammatory condition may alternatively involve local inflammation, including for example local internal inflammation, which may occur as a result of an autoimmune condition, e.g. as discussed further below, or local inflammation on an external surface of the body e.g. associated with a wound or trauma, or infection.

The invention includes also the treatment or prevention of “Low-level inflammation” (which term is used herein as synonymous with “low-grade inflammation”). This is a condition characterised by a 2- to threefold increase in the systemic concentrations of cytokines such as TNF-alpha, IL-6 and CRP, e.g. as measured in the plasma or serum. The increase may be relative to, or as compared with, normal concentrations or reference concentrations, for example concentrations as determined in a particular reference cohort or population of subjects, e.g. young subjects (e.g. young adults) or healthy subjects, for example subjects who are not suffering from any disease or condition, including any inflammatory condition, or who do not have inflammation. The increase may also be relative to the level of concentration in a subject prior to development of the inflammation. Low-level inflammation may be observed in the absence of overt signs or symptoms of disease. Thus, low-level inflammation may be sub-clinical inflammation. Alternatively, a subject with low-level inflammation may not have a clinically diagnosed condition or disease, but may exhibit certain signs or symptoms of an inflammatory response or inflammatory condition. In other words, there may be signs or symptoms of the effect of inflammation in the body, but this may not yet have progressed to an overt or recognised disease.

Exemplary inflammatory conditions include inflammatory bowel disease, osteoarthritis and other forms of arthritis, cancer-associated inflammation, cardiovascular diseases (i.e. CVD which is associated with inflammation or has an inflammatory component) and nephritis, as well as any inflammation associated with infection. Effectively, any inflammatory condition may be treated where CLPTM1 is expressed at the cell surface and is thus accessible to the binding agents of the invention, and where it is of clinical value to dampen the inflammation. As noted above, the binding agents used according to the invention may reduce inflammation of any cytokines regulated downstream of CLPTM1, and may thus more generally be used to treat any condition involving an undesirable or elevated cytokine profile.

The binding agents of the present invention may be of particular utility in the treatment or prevention of an autoimmune condition, (or alternatively termed, an autoimmune disease). Autoimmune diseases may be defined as any disease or condition which arises from an abnormal immune response in which a subject's immune system recognises and attacks ‘host’ cells and tissue from the subject.

Treatment of autoimmune conditions typically involves some degree of immunosuppression. In view of the discovery that binding agents for CLPTM1 may down-regulate certain immune responses in inflammatory disease model systems, such binding agents may be of benefit in the treatment of such diseases. Thus, although not limited to the following, autoimmune diseases which may be benefit from therapy from the binding agents of the present invention include conditions such as multiple sclerosis (MS), Crohn's disease, ulcerative colitis, systemic lupus erythematosus (SLE), ankylosing spondylitis, juvenile arthritis, rheumatoid arthritis, spondyloarthritis, psoriasis, systemic sclerosis (scleroderma), type 1 diabetes, polymyalgia rheumatica (PMR) and interferonopathies.

In a particular embodiment, a binding agent of the present invention may be used in combination with a TNF blocker (or TNFα blocker) in the treatment or prevention of an autoimmune or anti-inflammatory condition. In such a combination therapy, the binding agent and TNF-blocker may be provided together in a single composition (i.e. they may be co-formulated together in a pharmaceutical composition), or they may be provided separately, e.g. as a kit, or combination (e.g. a combined product) comprising the binding agent and TNF blocker. The binding agent and TNF blocker may be provided for simultaneous or sequential use. Such a combination may be used to treat any inflammatory or autoimmune condition defined or described herein, but especially in the treatment of prevention of arthritis, in particular rheumatoid arthritis. TNF blockers (also known as TNF inhibitors) are widely known and described in the art, and used clinically. Any such TNF blocker may be used, for example an anti-TNFα antibody (e.g. infliximab (Remicade) or adalimubab (Humira) or other anti-TNF monoclonal antibody), or receptor fusion protein (such as Etanercept (Enbrel (Pfizer)), or a small molecule (e.g. a xanthine derivative such as pentoxifylline or bupropion). Preferably, the TNFα blocker may be Etanercept (Enbrel (Pfizer)). In certain embodiments, the binding agent and the TNF blocker may exhibit a synergistic effect.

We have observed that IL-10 is markedly increased in response to a CLPTM1 binding agent. This indicates a utility of such binding agents in dampening autoimmune disorders, or indeed, more generally, any inflammatory condition. For example such an autoimmune disorder may affect the intestine or rectum—a binding agent capable of targeting the increased levels of CLPTM1 expressed on the cell surface in inflammation and inducing a high local level of IL-10 may have utility in treating autoimmune and/or inflammatory conditions of the GI tract, such as Crohn's Disease or ulcerative colitis, or more generally any inflammatory bowel disorder or localised inflammation in the GI tract (particularly the rectum or intestine) arising from whatsoever cause. Such a binding agent may for example be delivered locally (e.g., in an emulsion or other composition for topical or other administration to the rectum or intestine, or other site in the body, whether internal or external). Thus, advantageously, the binding agent may be administered locally, to treat local inflammation, including local internal inflammation, and thereby avoid or limit systemic effects.

In a particular embodiment, the binding agent of the present invention may be used in the treatment or prevention of multiple sclerosis.

We have identified that cytokines that are known to be dysregulated in MS patients may be altered by GDF15 and TGFβ, as well as the binding agents of the present invention. In particular, we have identified that such agents can increase secretion of Midkine, VEGF and ErbB4, all of which are known to be reduced or down-regulated in patients with MS. This suggests that the dys-regulation of cytokines in patients with MS may be reversed using the binding agents of the present invention, and highlights MS as a condition which might be particularly susceptible to treatment using the binding agents of the present invention.

In yet a further embodiment, a binding agent of the present invention may be used in combination with interferon β (IFN-β) in the treatment or prevention of multiple sclerosis, or more broadly in the treatment or prevention of an autoimmune or inflammatory condition. In such a combination therapy, the binding agent and IFN-β may be provided together in a single composition (i.e. they may be co-formulated together in a pharmaceutical composition), or they may be provided separately, e.g. as a kit, or combination (e.g. a combined product) comprising the binding agent and IFN-β. The binding agent and IFN-β may be provided for simultaneous or sequential use. Such a combination may be used to treat any inflammatory or autoimmune condition defined or described herein, but especially multiple sclerosis. In certain embodiments, the binding agent and the IFN-β may exhibit a synergistic effect.

In some subjects hypercytokinemia, which may be a result of a disease or condition as described herein, such as sepsis, malaria, graft versus host disease (GVHD) etc. can result in, or increase the risk of, multiple organ dysfunction syndrome (MODS), which is also known as multiple organ failure (MOF) or multisystem organ failure (MSOF). MODS, MOF or MOFS may therefore be treated or prevented using a binding agent of the present invention.

A particular condition which may be treated using a binding agent of the present invention is STING-associated vasculopathy with onset in infancy (SAVI). This disease is associated with a mutation in TMEM173/STING that leads to increased activation of the STAT1 pathway. Reducing the activity of TMEM/STING using a binding agent of the present invention may treat or prevent this condition.

An inflammatory response is induced via TMEM173/STING activation following radiotherapy treatment. Radiotherapy releases nucleic acids inside a cell, which can activate TMEM173/STING and result in activation of the immune system in and around a tumour. However, healthy cells that are harmed by radiotherapy treatment may also induce immune activation, which may cause, for example, radiation-induced dermatitis. Treatment using the binding agents of the present invention e.g. as a topically applied lotion or balm may prevent this.

The binding agents of the present invention may also reduce the activity of the immune system in a number of different ways to reduce the severity or risk of rejection following organ or tissue transplant. Transplant rejection occurs when transplanted tissue (e.g. a liver, kidney, heart, lung, cornea) is attacked by a recipient's immune system following a transplant. Conventionally this is treated using immunosuppressive agents, which have side-effects. As noted above and demonstrated further in the Examples below, exposure of immune cells to binding agents for CLPTM1 resulted in the down-regulation of interferon, and up-regulation of IL-10. Interferon is known to drive rejection following transplantation, and IL-10 is thought to have a protective (i.e. immunosuppressive) role in protection against transplant rejection. Furthermore, GDF15 has been found and reported to down-regulate MHC, which may reduce the extent to which transplanted tissue is attacked by a host immune system. Re-creating this effect using binding agents for CLPTM1 may therefore modulate the immune system in a number of different and complementary ways which may help reduce (i.e. treat) or prevent transplant rejection.

The immunosuppressive effects of the binding agents of the present invention may be of therapeutic use in other specific inflammatory diseases. For example, non-alcoholic Steatohepatitis (NASH) is a form of non-alcoholic fatty liver disease (NAFLD), defined by inflammation and fibrosis. NASH is progressive, and up to 20% of patients with NASH will develop cirrhosis of the liver over 10-year period, and 10% will suffer death related to liver disease. In NASH, hepatocyte mitochondrial DNA drives nonalcoholic steatohepatitis by activation of Toll-Like Receptor 9 (TLR-9). Disrupting TLR9 signalling using a binding agent of the present invention has the potential to break the circuitry in NASH.

In certain other embodiments, the binding agents of the present invention may find utility in down-regulating particular immune responses that are ‘hijacked’ by specific pathogenic microorganisms. For example, certain diseases are known to be associated and/or enhanced by elevated levels of type I interferon. Increased activity of TMEM173/STING may result in elevated levels of type I interferon, and thus targeting TMEM173/STING using a binding agent of the present invention may be of use in decreasing the levels of type I interferon. (Type I interferon includes IFN-α (alpha), IFN-β (beta), IFN-κ (kappa), IFN-δ (delta), IFN-ε (epsilon), IFN-τ (tau), IFN-ω (omega), and IFN-ζ (zeta, also known as limitin). Such a reduction may be viewed as a specific reduction in immune activity, allowing other components of a subject's immune system to combat the disease in question. Diseases associated with and/or enhanced by elevated levels of type I interferon may be therefore treated or prevented by the binding agents of the present invention. For example, several infectious diseases are known to hijack the response of a host immune system to type I interferon for their pathogenicity, such as intracellular infectious agents. In particular, in one example, the ESX1 secretion system of M. tuberculosis is known to induce phagocytosis by macrophages, allowing mycobacterial DNA to access the host cell's DNA sensors, inducing type I interferon secretion. Elevated levels of type I interferon are known to result in increased M. tuberculosis pathogenicity and prolonged infection. In another example, TMEM173/STING-mediated type I interferon response has been central to the pathogenesis of cerebral malaria in laboratory animals infected with Plasmodium berghei, and laboratory animals deficient in type I interferon response have been found to be resistant to cerebral malaria. Contacting CLPTM1 with a binding agent of the present invention would result in the down-regulation of type I interferon, and may therefore help treat or prevent infections with intracellular pathogens (e.g. malaria) and other such conditions.

More generally, type I interferonopathies represent a class of condition which may be treated or prevented according to the present invention. Type I interferonopathies include Alcardi-Goutière Syndrome (AGS), conditions due to mutations in the gene IF1H1 (coding for a cytosolic double-stranded RNA receptor protein, MDAS, familial chilblain lupus (FCL), spondyloenchondromatosis (SPENCO), Singleton-Merton Syndrome (SMS), SAVI (mentioned above), and chronic atypical neutrophilic dermatosis (CANDLE) in children and JNP in juveniles (joint contractures, muscle atrophy, microcytic anaemia, and panniculitis-induced lipodystrophy) syndromes, which are autosomal-recessive auto-inflammatory conditions.

Variants of the STING protein as well as dysregulated STING signalling have been implicated in a variety of inflammatory diseases as discussed for example by Li et al., in Journal of inflammation, 2017, 14:11. All such diseases, that is, conditions associated with dysfunction of STING, are candidates for treatment and prevention according to the present invention. This may include autoimmune conditions as discussed above, auto-inflammatory disease, and microbial infections, e.g. as discussed above, as STING is known to be activated by a number of pathogens, including viruses (for example single-stranded RNA viruses, such as Sendai virus and vesicular stomatitis virus (VSV), and double-stranded DNA viruses, including non-enveloped and enveloped double-stranded DNA viruses, e.g. HIV, Adenovirus, HSV, CMV, and Epstein-Barr virus), and bacteria including Streptococcus sp., Staphylococcus sp., Listeria monocytogenes, Vibrio cholerae and Mycobacterium tuberculosis. Thus, viral and bacterial infections are candidate conditions. STING may also regulate lipid metabolism, and therefore conditions involving altered lipid metabolism are also included.

The immunosuppressive effects of the binding agents of the present invention may further be of utility in reducing the activity of the immune system in haematological disorders, including haematopoietic cancers. Haematopoietic cancers (e.g. leukemias and lymphomas) are notably associated with low levels of GDF15 and TGF-β3. Without wishing to be bound by theory, this may place the immune system in a pro-inflammatory state, which drives or maintains an autocrine or paracrine feedback loop. In such a setting, the a binding agent of the present invention could mimic the downstream effect of these proteins and relieve the pro-inflammatory state of the immune system as discussed above by acting as a ‘brake’ by inactivating TMEM173/STING.

The term “haematopoietic cancer” includes any cancer of the blood or bone marrow, and includes both acute and chronic leukemia, and lymphomas. Leukemia may be further categorised as lymphoblastic (lymphocytic) and myelogenous. Acute lymphoblastic leukemia includes precursor B acute lymphoblastic leukemia, precursor T acute lymphoblastic leukemia, Burkitt's leukemia and acute bihphenoid leukemia. Chronic lymphocytic leukemia includes B-cell prolymphocytic leukemia. Acute myelogenous leukemia includes acute promyelocytic leukemia, acute myeloblastic leukemia, and acute megakaryoblastic leukemia. Chronic myelogenous leukemia includes chronic myelomonocytic leukemia. Further forms of leukemia include hairy cell leukemia (affecting B-cells), T-cell prolymphocytic leukemia, large granular lymphocytic leukemia (affecting T-cells or NK cells) and adult T-cell leukemia. Lymphomas include Hodgkin lymphoma (including nodular sclerosis, mixed cellularity Hodgkin lymphoma, nodular lymphocyte-predominant Hodgkin lymphoma) and non-Hodgkin lymphomas, small cell lymphoma, splenic marginal zone lymphoma, MALT lymphoma, nodal marginal zone B lymphoma, follicular lymphoma, primary cutaneous follicle centre lymphoma, mantle cell lymphoma, diffuse large B cell lymphoma, diffuse large B-cell lymphoma associated with inflammation, lymphomatoid granulomatosis, primary mediastinal large B-cell lymphoma, intravascular large B-cell lymphoma, plasmablastic lymphoma, primary effusion lymphoma, Burkitt lymphoma, Waldenström macroglobulinemia (WM), B-cell lymphoma (CBCL), lymphoplasmacytic lymphoma (LPL), marginal zone lymphoma (MZL), adult T-cell lymphoma, extranodal NK/T-cell lymphoma, tenteropathy-associated T-cell lymphoma, hepatosplenic T-cell lymphoma, blastic NK cell lymphoma, peripheral T-cell lymphoma, agioimmunoblastic T-cell lymphoma, anaplastic large cell lymphoma and primary CNS lymphoma (PCNSL).

In a particular embodiment the haematopoietic cancer may be associated with the MYD88L265P mutation which is typically found in WM (it occurs in almost all cases of WM) (Wang et al. 2016. Blood). This mutation may also occur in other haematopoietic cancers including, LPL (over 90%), CBCL (30-40%), and in rare cases of MZL (2-4%), and CLL. We have observed that this mutation is associated with an increased expression of CLPTM1 at the cell surface (as can be seen when HEK293 cells are transfected with the receptor, where a 3.5 fold increase of cell surface CLPTM1 was seen). Thus in haematological disorders where the MYD88 pathway is over-activated, such as occurs in connection with the MYD88L265P mutation, the use of a CLPTM1 binding agent may induce the “brake” upstream of MYD88, reducing the overall activity of TLRs, thereby altering the downstream signalling and secretome, and reducing the pathological axis. In such disorders the gain of function is cell-specific (i.e. in the tumour cells), and thus the tumour cells which over-express CLPTM1 may be specifically targeted by the binding agent of the present invention.

Other inflammatory diseases may also be treated according to this aspect of the present invention. As noted above, PTP1B inhibition or knockout has been found to increase the secretion of placental growth factor (PIGF). Elevated levels PIGF have been found to protect placenta during preeclampsia, and thus enhancing PTP1B degradation using binding agents of the present invention may represent a possible treatment for preeclampsia.

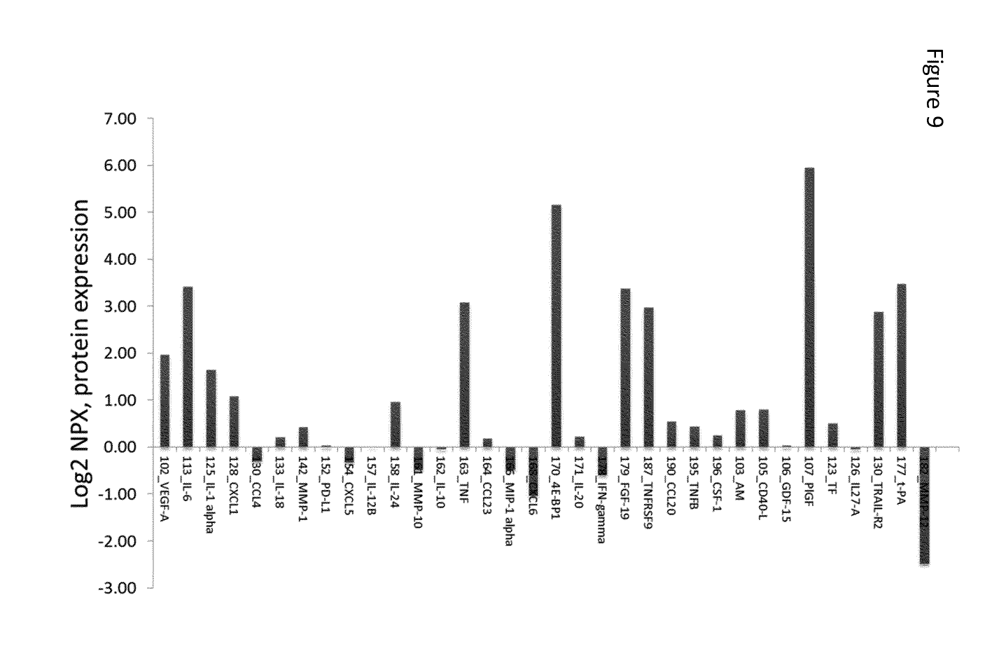

It has previously been also reported that loss of active TGF-β results in elevated expression of matrix metalloproteinase 12 (MMP12) (Morris et al. 2003. Nature 422, 169-173). MMP12 degrades elastin and is a key effector of emphysema and other lung disorders, such as COPD more generally. We have found that MMP12 is reduced by anti-CLPTM1 antibodies in the experiments performed in the process of making the present invention (see FIG. 9). Accordingly, lung disorders including specifically emphysema and COPD may therefore be treated using the binding agents of the present invention.

The binding agents of the present invention may also be used to treat or prevent coronary artery disease, The term ‘coronary artery disease’ (also known as ischemic heart disease) refers to diseases defined by narrowing of the coronary artery and luminal narrowing. In particular, the binding may be used to reverse, reduce or prevent the accumulation of atherosclerotic plaques which reduce the luminal area of coronary arteries, or in other words the binding agents may be used in the treatment or prevention of atherosclerosis.

Atherosclerosis, also known as arteriosclerotic vascular disease (ASVD) is a specific form or arteriosclerosis, a family of diseases characterised by thickening, hardening and loss of elasticity in the walls of arteries. Atherosclerosis is defined by thickening of the artery wall as a result of the formation of a fibrofatty plaque, formed by the accumulation of fatty deposits on the wall of an artery, and subsequent engulfment of the fatty deposit by foam cells (a form of fat-laden macrophage), coupled with proliferation of intimal-smooth-muscle cells. Atherosclerosis is therefore a syndrome affecting arterial blood vessels due to a chronic inflammatory response of white blood cells, particularly macrophages, in the walls of arteries

We have identified that GDF15 stimulation of immune cells results in increased CXCL5 expression. This cytokine is known to prevent the formation of foam cells (i.e. the engulfment of fatty deposits by white blood cells) which lead to the atherosclerotic plaques. Thus, the use of a CLPTM1 binding agent of the present invention represents a possible treatment by reducing inflammation at the site of an atherosclerotic plaque.

The present invention therefore provides binding agents capable of binding to CLPTM1, for use in treating or preventing any of the above-mentioned conditions.

Also provided are methods of treating the above-mentioned conditions, said methods comprising administering to a subject in need thereof a therapeutically effective amount of a binding agent capable of binding to CLPTM1.

In yet another aspect, the present invention also provides the use of a binding agent capable of binding to CLPTM1, in the manufacture of a medicament for treating or preventing any of the above-mentioned conditions.

In particular embodiments of these aspects, the conditions include NASH, nephritis, preeclampsia, haematological disorders, particularly haematological cancers, lung disorders, including particularly emphysema or COPD, an infection with an intracellular pathogen, transplant rejection, GvHD, MODS, MOF or MOFS, inflammation in the GI tract such as Crohn's disease, ulcerative colitis or other inflammatory bowel disease and coronary heart disease or atherosclerosis, or the binding agent of this aspect of the invention may be for use as a cardioprotective agent.

In another aspect of the present invention, CLPTM1 binding agents may increase insulin sensitivity, or alternatively expressed, may reduce insulin resistance. As discussed above, the binding agents of the present invention may enhance the degradation of PTP1B through CLPTM1. Dephosphorylation of the insulin receptor by PTP1B is known to reduce insulin sensitivity, and thus the binding agents of the present invention may be used to reduce PTP1B levels, thereby improving or increasing sensitivity to insulin, or alternatively expressed, reducing or overcoming insulin resistance. Furthermore, we have determined that a CLPTM1 binding agent may result in up-regulation of FGF19-FGF19 has been implicated as a key metabolic factor with the potential to aid in insulin resistance/diabetes pathology. This, thus, provides a rationale for treating or preventing insulin resistance or conditions associated therewith with a CLPTM1 binding agent according to the present invention. Such conditions include notably metabolic syndrome and type 2 diabetes, but may also include other metabolic disorders. Any metabolic disorder associated with insulin resistance or having insulin resistance as a component may be treated or prevented according to the present invention. This may include also NAFLD. According to this aspect of the present invention, metabolic diseases more broadly may be treated or prevented according to the present invention. Metabolic syndrome, defined by high blood pressure, decreased fasting serum HDL, elevated fasting serum triglyceride (VLDL), impaired fasting glucose, and insulin may therefore be treated or prevented according to the present invention. Associated conditions such as obesity, hyperuricemia, and PCOS may also be treated or prevented according to this aspect of the invention.

Accordingly, the present invention therefore provides a binding agent capable of binding to CLPTM1, for use in the treatment or prevention of insulin resistance or a condition associated therewith (or a condition associated with reduced sensitivity to insulin). In particular, the disease may be metabolic syndrome or type 2 diabetes.

Also provided is a method of treating or preventing insulin resistance or a condition associated therewith (or a disease associated with reduced sensitivity to insulin), in particular type 2 diabetes or metabolic syndrome, said method comprising administering to a subject in need thereof an effective amount of the binding agent capable of binding to CLPTM1.

The present invention also provides the use of a binding agent capable of binding to CLPTM1, in the manufacture of a medicament for treating or preventing insulin resistance or a condition associated therewith (or a disease associated with reduced insulin sensitivity), in particular type 2 diabetes or metabolic syndrome.

In yet another aspect of the present invention, the binding agents may be used to treat, prevent or reduce damage to the heart, more particularly damage caused by injury or disease. Accordingly the agents may be used as cardioprotective agents, or alternatively expressed, for cardioprotection. The term “cardioprotection” as used herein refers to an effect of protecting the heart from damage or of treating or alleviating damage to the heart (or more particularly of treating or alleviating the effects of damage to the heart). A “cardioprotective agent” thus mediates or has a cardioprotective effect. “Protection” encompasses both preventing and limiting or reducing damage. Thus, absolute prevention of damage is not required, and protection may be seen as any degree of reduction in damage incurred (for example as compared with a subject or individual who has not been treated with (e.g. administered) the cardioprotective agent). In one aspect, cardioprotection may thus be seen as rendering the heart less susceptible to damage. The amount or degree of damage may be limited or reduced, or in some cases damage may be prevented altogether. Alternatively, a cardioprotective agent may exert a therapeutic effect on damaged heart tissue. Thus, cardioprotection includes also an effect in alleviating or mitigating the effects of damage on the heart, or of reducing or ameliorating the damage.

“Damage” to the heart includes any effect on the heart which impedes it from working normally (or properly), i.e. which prevents or reduces cardiac function, or which causes the heart to function in a reduced or less effective way. Thus, damage may be any effect which results in cardiac dysfunction.

In particular, the damage to the heart is myocardial damage, in other words damage to the myocardium or to cardiac myocytes (cardiomyocytes). Damage to the heart may be seen as cellular damage, for example mitochondrial damage or damage to other sub-cellular organelles or structures, cell death, apoptosis, necrosis or infarction, or as hypertrophy of the heart or a change in cardiac structure, geometry, size or dimensions (e.g. remodelling of the heart). Damage to the heart may include ischaemic damage, namely damage (particularly cellular damage) seen as result of ischaemia, or damage due to reperfusion after ischaemia (ischaemia/reperfusion injury), or damage resulting from increased cardiac workload or cardiac stress, particularly chronic cardiac stress or a chronic increase in cardiac workload, damage resulting from cardiotoxic substances (e.g. chemotherapeutic drugs such as doxorubicin or anthracyclines, chronic alcohol abuse, or heavy metals, including iron and copper), as well as damage resulting from infection, e.g. viral infection, leading to myocarditis (e.g. cytomegalovirus or coxsackie virus).

Ischaemic damage or ischaemia/reperfusion injury may occur in ischaemic heart disease, e.g. coronary artery disease, and particularly in acute coronary syndromes (e.g. a myocardial infarction (MI), or during surgery, for example surgery on the heart or when a patient is on a heart-lung machine.

Damage resulting from cardiac stress or increased cardiac workload may occur in heart or cardiovascular disease or injury and may result from any condition (i.e. disease or disorder) which increases the pressure on the heart (pressure overload), for example hypertension or aortic stenosis. In this setting, the myocardial tissue remaining viable after myocardial infarction may also be subject to increased workload due to increased peripheral vascular resistance.

PTP1B has been found to be widely expressed in cardiovascular tissues, including the heart and endothelium and its degradation has been suggested to be a possible therapy in several cardiovascular diseases. Gene deletion of PTP1B reduces endothelial dysfunction in various cardiovascular diseases and blocking PTP1B increases cardiac angiogenesis (partly through increased VEGFR activity as described above) (Thiebault et al. 2016. J Mol Cell Cardiol. Role of protein tyrosine phosphatase 1B in cardiovascular diseases (in press)). PTP1B inhibition or gene deficiency has also been found to reduce heart failure (Gomez et al. 2012. J Mol Cel Cardiol. 52, 1257-1264). GDF15 has previously been found to protect against damage to cardiac myocytes during acute myocardial infarction (AMI). Without wishing to be bound by theory, it is thought that this effect is achieved through GDF15 binding to CLPTM1. Binding of binding agents for CLPTM1 to this receptor is believed to mimic the downstream effects of this role of GDF15, and thus the binding agents of the present invention may be used to treat or prevent damage to the heart, for example following AMI.

The binding agents may thus be used in the treatment or prevention of myocardial damage. The damage may arise from ischaemia, ischaemia reperfusion injury, hypoxia, increased cardiac workload or cardiac stress, increased pressure on the heart, cardiotoxic substances, infection, or a maladaptive response of the heart to injury or disease. In further embodiments the binding agents may be used in the treatment or prevention of heart failure, or a condition or disease which predisposes or leads to heart failure, or an acute coronary syndrome, or to protect the heart during or after surgery. In other embodiments the binding agents may be used in the treatment or prevention of ischaemic heart disease, cardiomyopathy, ventricular dysfunction, ventricular remodelling, unstable angina or myocardial infarction, or to protect an explanted heart during ex vivo transportation. More particularly, the binding agents may be used:

(i) in the treatment of chronic heart failure, or

(ii) to prevent or delay the onset or development of heart failure, or

(iii) to protect the heart from damage from a coronary event; or

(iv) to protect the heart from damage due to hypertension or aortic constriction; or

(v) to precondition the heart to protect from reperfusion injury following ischaemia and subsequent restoration of blood flow; or

(vi) to prevent or delay the onset or development of heart failure after myocardial infarction (MI); or

(vii) to prevent or reduce the extent of MI; or

(viii) to pre-condition the heart to prevent or reduce damage during or after surgery; or

(ix) before, during or after percutaneous coronary intervention (PCI).

The present invention therefore provides a binding agent capable of binding to CLPTM1 for use as a cardioprotective agent (i.e. in treating, preventing or reducing damage to cardiomyocytes).

Also provided is a method of cardioprotection (i.e. a method of treating, preventing or reducing damage to cardiomyocytes), said method comprising administering to a subject in need thereof an effective amount of the binding agent capable of binding to CLPTM1.

Use of a binding agent capable of binding to CLPTM1, in the manufacture of a cardioprotective agent (i.e. a medicament for use as a cardioprotective agent, or for treating, preventing or reducing damage to cardiomyocytes) is also provided.

In another aspect, the present invention provides use of the binding agent as defined herein in binding to CLPTM1 in vitro.

Thus, according to this aspect the invention also provides a method for inhibiting (or down-regulating) the CLPTM1 receptor in vitro, said method comprising contacting cells (e.g. a culture or preparation of cells, or a tissue ex vivo) expressing CLPTM1 (particularly cells expressing CLPTM1 on the cell surface) with a binding agent as defined herein. As noted above, the binding agents may bind to CLPTM1 and thereby inhibit or inactivate it, or reduce its effects. Since CLPTM1 appears to have an inhibitory role in down-regulating cell signalling, and thereby promoting immune activity (or reducing immunosuppression), a binding agent may act to down-regulate (block or inhibit) the inhibitory effect of CLPTM1, thereby achieving or promoting an immunosuppressive (or immune-inhibitory) effect. The binding agents effectively act as an inhibitor (or antagonist) of an inhibitory receptor and thereby promote or induce downstream effects which are inhibited by the receptor.

The term “treating” is used broadly herein to include any aspect of improving or ameliorating a condition or the clinical status of a subject suffering from or having the condition. Thus, a complete cure of the condition is not required and “treating” includes improving any aspect, parameter or symptom of a condition.

Similarly, the term “preventing” is used broadly herein to include any aspect of reducing or delaying a condition, or the onset or progression of a condition. Thus preventing does not require complete or absolute prevention of the development of a condition and may include delaying or slowing the progression or onset of any aspect, symptom or parameter of a condition. The severity of a symptom, parameter or aspect may be reduced and/or it may be delayed in developing. In a particular embodiment, the progression or development of one or more symptoms of a condition or disease may be delayed, reduced or prevented.

The present invention accordingly provides binding agents which are capable of binding to CLPTM1 in a subject, thereby mimicking a downstream effect of GDF15 or TGF-β (which may be natural ligands for CLPTM1, although it is not precluded that they may exert their effects by other mechanisms).

The binding agents of the present invention are capable of binding to CLPTM1 and, in some embodiments, inducing a downstream effect as described above that is normally associated with a natural ligand for CLPTM1, such as GDF15 or TGFβ. Binding agents are not limited to agents which bind to a receptor in the same manner as the natural ligand for a receptor, and include agents which bind to the receptor in any manner in order to effect its physiological function. Binding agents according to the present invention therefore include any agent which is capable of inducing a physiological effect on binding to the receptor, and may include any agent which binds to the receptor and induces such an effect at a detectable level. Binding agents which bind the receptor and induce one or more effects with partial or full efficacy relative to a natural ligand, or which bind to a receptor to produce a greater maximal response than the endogenous ligand for the target receptor. Binding agents may therefore have at least 10% of the efficacy of the natural ligand, but more preferably with have at least 20%, 30%, 40%, 50%, 60%, 70%, 80%, 90% or 100% of the efficacy of the natural ligand. In further preferred embodiments, a binding agent will have at least 110%, 120%, 130%, 140% or 150%, or 200%, 250%, 300%, 350% or 400% of the efficacy of the natural ligand.

As noted above, CLPTM1 is a transmembrane protein with a ˜350 amino acid extracellular domain. The amino acid sequence of this receptor in humans is shown in SEQ ID NO:1. The binding agents of the present invention bind to an accessible portion of CLPTM1, i.e. to the extracellular domain of CLPTM1. The extracellular domain of CLPM1 has 353 amino acids (representing amino acids 2-353 of SEQ ID NO:1) and is shown in SEQ ID NO:2.

A binding agent of the invention may bind to any part (i.e. any site in) the ECD of CLPTM1, and not necessarily the same binding site as a natural ligand. Thus in certain embodiments the binding agent may bind to all or any part of an amino acid sequence as shown in SEQ ID NO.2 or an amino acid sequence having at least 80% sequence identity thereto (e.g. at least 85, 90 or 95% sequence identity thereto).

Sequence identity may readily be determined by methods and software known and readily available in the art. Thus, sequence identity may be assessed by any convenient method. However, for determining the degree of sequence identity between sequences, computer programs that make multiple alignments of sequences are useful, for instance Clustal W (Thompson et al., (1994) Nucleic Acids Res., 22: 4673-4680). Programs that compare and align pairs of sequences, like ALIGN (Myers et al., (1988) CABIOS, 4: 11-17), FASTA (Pearson et al., (1988) PNAS, 85:2444-2448; Pearson (1990), Methods Enzymol., 183: 63-98), BLAST and gapped BLAST (Altschul et al., (1997) Nucleic Acids Res., 25: 3389-3402) are also useful for this purpose, and may be used using default settings. Furthermore, the Dali server at the European Bioinformatics institute offers structure-based alignments of protein sequences (Holm (1993) J. Mol. Biol., 233: 123-38; Holm (1995) Trends Biochem. Sci., 20: 478-480; Holm (1998) Nucleic Acid Res., 26: 316-9). Multiple sequence alignments and percent identity calculations may be determined using the standard BLAST parameters, (using sequences from all organisms available, matrix Blosum 62, gap costs: existence 11, extension 1). Alternatively, the following program and parameters may be used: Program: Align Plus 4, version 4.10 (Sci Ed Central Clone Manager Professional Suite). DNA comparison: Global comparison, Standard Linear Scoring matrix, Mismatch penalty=2, Open gap penalty=4, Extend gap penalty=1. Amino acid comparison: Global comparison, BLOSUM 62 Scoring matrix. Variants of the naturally occurring polypeptide sequences as defined herein can be generated synthetically e.g. by using standard molecular biology techniques that are known in the art, for example standard mutagenesis techniques such as site-directed or random mutagenesis (e.g. using gene shuffling or error prone PCR).

In the work leading up to the present invention, the binding sites for GDF15 was identified in a peptide binding array, and the results of such a study is provided below in Example 2. A proposed binding site for GDF15 are described below and shown in SEQ ID NO: 18, 19, 22, 23, 29 and 30 corresponding to amino acids 162-181; 182-201; 242-261; 262-281; 292-311; and 273-292 of SEQ ID NO:1, respectively. In certain embodiment, the binding agents of the present invention may bind to an epitope within CLPTM1 which partially or fully overlaps with the binding epitope of GDF15. Thus, in one embodiment, the binding agent may bind to a polypeptide having an amino acid sequence as set forth in any one of SEQ ID NOs:34, 35, 36, 37, 38 or 39, which correspond to the peptides used in this experiment, but do not comprise C-terminal glycine residues (i.e. they represent the sequences from CLPTM1).

However, as noted above, it is not essential that the binding agents of the present invention bind to the same site within CLPTM1 as one or any of the native ligands for the receptor, and thus in other embodiments the binding agent does not bind to a binding site in CLPTM1 for a native ligand. Indeed, we have shown that binding agents which bind to CLPTM1 and which may be useful according to the present invention, notably antibody-based binding agents, may bind to various different regions of CLPTM1, and in particular throughout the ECD of CLPTM1.

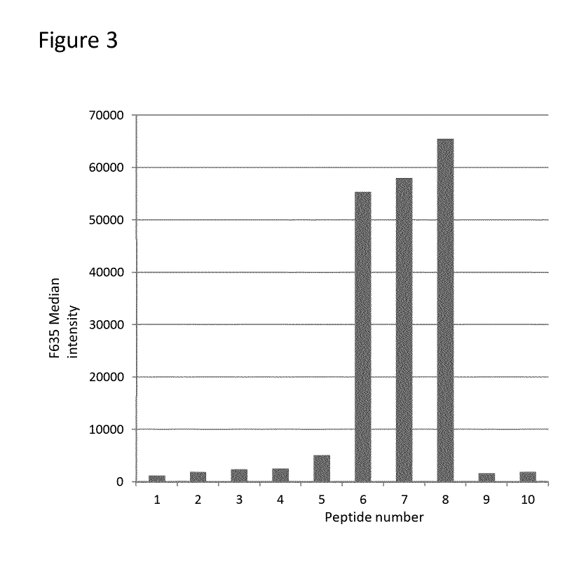

In the work leading to the present invention, we have identified an antibody which binds to an amino acid sequence situated toward the N-terminus of CLPTM1. The characterisation of the binding site is shown in FIG. 3 and Example 3 below. Accordingly, in a particular embodiment, the binding agent may bind to a polypeptide having an amino acid sequence as set forth in SEQ ID NO: 3. In further embodiments, the binding agent may also bind to a broader binding site having an amino acid sequence as set forth in SEQ ID NO:4, or to any of the peptides A5-A9 (see Table 2) represented by SEQ ID NOs:5-9.

We have also generated a mouse monoclonal antibody (7G12 in the Examples) which binds to a different sequence situated towards the C-terminus of the extracellular domain of CLPTM1. A peptide having the sequence set forth in SEQ ID NO: 40, corresponding to amino acids 326-340 of CLPTM1, was used to inoculate mice to generate antibodies for CLPTM1. The sequences of the variable heavy and light domains of an antibody generated against this peptide are provided as SEQ ID NOs:44 (IgG1) and 45 (Kappa). A further antibody (59D04 in the Examples) has also been identified by a phage display screen using a peptide target portion of CLPTM1 which encompasses SEQ ID NO:43. This longer peptide, set forth in SEQ ID NO:43, corresponds to amino acids 312-354 of CLPTM1. This antibody comprises variable heavy and light domains as set forth in SEQ ID NOs:41 and 42, respectively. As described in greater details below, these antibodies have been observed to have measurable effects both in in vivo and in in vitro studies. Accordingly, in certain embodiments, the binding agent may bind to a polypeptide having an amino acid sequence as set forth in SEQ ID NOs:40 or 43. The effect of antibodies capable of binding to peptides having sequences as set forth in SEQ ID NOs:48 and 49 was also assessed in the Examples, and in yet further embodiments, the binding agent for CLPTM1 described herein may bind to either of these peptides.

The term “binding agent” may refer to any agent e.g. any compound or molecule or entity having the ability to bind to CLPTM1, in particular to an extracellular domain of CLPTM1. In particular, the binding agent may bind specifically to CLPTM1. By binding specifically is meant that the agent is capable of binding to CLPTM1 in a manner which distinguishes it from binding to a non-target molecule. Thus, binding to a non-target molecule may be negligible or substantially reduced as compared to binding to CLPTM1. A binding agent may thus be any agent having a binding affinity for CLPTM1 i.e. an affinity binding partner for CLPTM1, or more particularly for the extracellular domain thereof.

Thus, in a particular embodiment, the binding agent is specific for CLPTM1. Specific binding agents advantageously would target only those signalling pathways that are linked to CLPTM1, as they would have no or low cross-reactivity with other receptors for GDF15 or TGF-β, and would therefore advantageously have more limited side effects when used in therapy over binding agents which resembled the natural CLPTM1 ligands GDF15 or TGF-β. In this way, the systemic activation of pathways associated with either or both of these proteins may be avoided, and a more targeted response may be achieved.

The binding agent may be any proteinaceous or non-proteinaceous molecule but, as discussed further below, will be preferably be an antibody. Binding agents of the present invention may thus be selected from proteins or polypeptides such as antibodies or fragments or derivatives thereof, a combinatorially derived polypeptide from phage display or ribosome display or any other peptide display system, or a nucleic acid molecule, such as an aptamer, or combinations thereof.

In a preferred embodiment of the invention, the binding agent is a protein, preferably an antibody or derivative or fragment thereof. Various antibody-like molecules are also known and described in the art and may be used, e.g. affibodies and such like.

In a preferred embodiment, the binding agent is an antibody. The antibody may be of any convenient or desired species, class or sub-type. Furthermore, the antibody may be natural, derivatised or synthetic. The term “antibody” as used herein thus includes all types of antibody molecules and antibody fragments.

The present invention therefore provides an antibody capable of binding to CLPTM1 for use as defined and described herein.

More particularly the “antibody” according to the present invention includes:

(a) any of the various classes or subclasses of immunoglobulin e.g. IgG, IgG1, IgG2, IgG3, IgG4, IgA, IgM, IgD or IgE derived from any animal e.g. any of the animals conventionally used e.g. sheep, rabbits, goats, or mice or egg yolk;

(b) monoclonal or polyclonal antibodies;

(c) intact antibodies or fragments of antibodies, monoclonal or polyclonal, the fragments being those which contain the binding region of the antibody e.g. fragments devoid of the Fc portion (e.g. Fab, Fab′, F(ab′)2, Fv), the so called “half molecule” fragments obtained by reductive cleavage of the disulphide bonds connecting the heavy chain components in the intact antibody. Fv may be defined as a fragment containing the variable region of the light chain and the variable region of the heavy chain expressed as two chains;

(d) antibodies produced or modified by recombinant DNA or other synthetic techniques, including monoclonal antibodies, fragments of antibodies, humanised antibodies, chimeric antibodies, or synthetically made or altered antibody-like structures. Also included are functional derivatives or “equivalents” of antibodies e.g. single chain antibodies.

A single chain antibody may be defined as a genetically engineered molecule containing the variable region of the light chain, the variable region of the heavy chain, linked by a suitable polypeptide linker as a fused single chain molecule. Single chain antibodies include ScFv antibodies.

Methods of making such antibody fragments and synthetic and derivatised antibodies are well known in the art. Also included are antibody fragments containing the complementarity-determining regions (CDRs) or hypervariable regions of the antibodies. These may be defined as the region comprising the amino acid sequences on the light and heavy chains of an antibody which form the three dimensional loop structure that contributes to the formation of the antigen binding site. CDRs may be used to generate CDR-grafted antibodies. As used herein “CDR grafted” defines an antibody having an amino acid sequence in which at least parts of one or more sequences in the light and/or variable domains have been replaced by analogous parts of CDR sequences from an antibody having a different binding specificity for a given antigen. One of skill in the art can readily produce such CDR grafted antibodies using methods well known in the art.

A chimeric antibody may be prepared by combining the variable domain of an anti-receptor antibody of one species with the constant regions of an antibody derived from a different species. Such techniques may be used to humanise antibodies for therapeutic use.

Monoclonal antibodies and their fragments and derivatives are preferred antibodies according to the present invention.

Preferably the antibody will be a humanised or chimeric antibody, i.e. an antibody which has been modified or created to comprise a binding domain (e.g. a complementarity determining region (CDR)) which recognises human CLPTM1 and a fixed (e.g. Fc) domain that has been modified to increase their similarity to antibody variants produced naturally in humans. In a particular aspect, a humanised or chimeric monoclonal antibody may be the IgG4 subtype. Modifications of this type are desirable to provide antibodies which have a minimal immunogenic effect.

In another preferred embodiment the antibody may be a human antibody. Human antibodies may be prepared using transgenic mice or other transgenic animals which may have been modified to express human immunoglobulin genes. They may also be obtained from phage display or indeed they may be isolated from human subjects, namely a human subject in whom the anti-receptor antibodies natively exist or are present (i.e. without the need to immunise the subject for antibody production), for example a human auto-immune subject.

Antibodies binding to CLPTM1 have been reported and are commercially available (see for example in the Examples below). One such example is rabbit monoclonal EPR8800 available under the name RabMab® from Abcam, Cambridge, UK, the binding site for which is elucidated in Example 3 below. Furthermore, antibodies to the receptors may readily be prepared using known and routine techniques. Methods for determining the binding site of an antibody in its antigen (the moiety to which it binds) are also routine (see also the Examples below) and so it is readily possible to determine the epitope, or antibody binding site in CLPTM1.

As described above, other antibodies which bind to the extracellular domain of CLPTM1 have been generated in the course of making the present invention, and the binding agent of the present invention may be an antibody comprising the variable regions of such antibodies. Thus, in a particular embodiment, the binding agent may be an antibody comprising the variable portions of their heavy and light chains as set forth in SEQ ID NOs:44 and 45 or 41 and 42, respectively.

In certain other embodiments the binding agent may be or comprise all or a portion of one of the natural ligands for CLPTM1. Thus, in one embodiment the binding agent may be or comprise GDF 15 or a TGF-β protein, e.g. TGF-β1, 2 or 3 or a derivative or fragment thereof, or a fusion protein comprising a GDF15 or a TGF-β or such a derivative or fragment, could be used as a binding agent of the present invention. However, it is preferred that the binding agent is not or does not comprise a natural ligand for CLPTM1, and thus in certain embodiments, the binding agent is not or does not comprise GDF15 or a TGF-β, or a derivative or fragment thereof.

Without wishing to be bound by theory, it is possible that binding agents having any binding affinity may bind to CLPTM1 and to a greater or lesser effect induce the effects which have been observed. However, we believe that a certain level of affinity may be required in order to achieve the effects at a level which is useful and clinically relevant, and that antibodies having a low affinity may not achieve observable, or relevant, effects, i.e. they may not be effective. In particular, we have observed that the efficacy of a binding agent correlates with its affinity for CLPTM1, and thus binding agents having low affinity for CLPTM1 are not believed to be able to induce the effects described herein at a sufficient level to be physiologically relevant. The present invention therefore utilises binding agents having a high affinity for CLPTM1, or in other words, high-affinity binding agents for CLPTM1. In the context of the present invention, the term “high-affinity” therefore refers to binding agents which are capable of binding to CLPTM1 and inducing one or more physiological changes which are described herein, and which may normally be associated with a natural ligand for CLPTM1, such as GDF15 or TGFβ. In particular, a high affinity antibody is capable of inducing a change in the cytokine profile, as described herein.

By measuring the binding affinities and efficacies of a number of different binding agents (more specifically, antibodies), it has been possible to identify a threshold binding affinity which a binding agent requires in order to be effective in inducing the effects described herein. A high affinity antibody is an antibody which meets this affinity threshold (affinity binding requirement).

The binding affinity of an antibody may be measured in any of a variety of different ways, including by various biophysical means such as Surface Plasmon Resonance (SPR) or Isothermal Titration calorimetry (ITC), or by spectroscopic means such as NMR shift-mapping in order to calculate a K_{D} value for a binding agent.

Alternatively, a dilution series of the binding agent may be prepared and binding of the binding agent to CLPTM1 may be measured for the each concentration of the binding agent, thereby to determine the concentration at which 50% of the maximal binding is achieved, i.e. measure an EC₅₀ value for a binding agent. Binding in such an assay may be measured in a variety of convenient, ways, including by ELISA, e.g. by immobilising CLPTM1 on the surface of wells of a multi-well plate and measuring the extent of binding of the binding agent at each different concentration, or by flow cytometry, by measuring the extent of binding of the binding agent to cells expressing CLPTM1. In each case, binding of the binding agent to CLPTM1 may be measured directly, e.g. using a binding agent that has been labelled with a detection label, or indirectly, e.g. using a secondary detection reagent, e.g. a secondary antibody, which carries such a label (the literal meaning of the term “secondary” antibody in this context refers to embodiments of the invention in which the detection agent is an antibody, but it should be understood that an equivalent detection assay may be performed for other binding agents using an antibody which carries a detection label, and thus that this term should not be considered to be limiting on the nature of the binding agent).

Accordingly, the binding agents which are to be used according to the present invention (e.g. which may be termed “high affinity”) have an EC₅₀ value of 1 μg/ml or less when determined by measuring binding to membrane-permeabilised 0-876 cells expressing native CLPTM1 by flow cytometry. More particularly, however, the EC₅₀ value when measured in this way is 0.9, 0.8, 0.7, 0.6, 0.5, 0.4, 0.3, 0.2 or 0.1 μg/ml or less. In representative particular embodiments, the EC₅₀ value when measured in this way is 0.5 or 0.1 μg/ml or less.

In experiments described in the Examples below, antibody 7G12 was found to demonstrate greater than 50% maximal binding at 1 μg/ml. The EC₅₀ value is accordingly lower than 1 μg/ml. Thus, the threshold for affinity of the binding agent according to the invention may be set at less than 1 μg/ml.

Alternatively, the affinity of the binding agent may be defined on the basis that the binding agent demonstrates greater than 50% maximal binding at 1 μg/ml or less when determined by measuring binding of the binding agent to membrane-permeabilised 0-876 cells expressing native CLPTM1 by flow cytometry. In other words the affinity may be defined as a value at which the binding agent shows greater than 50% of the maximal binding in the flow cytometry assay defined above. In particular embodiments, the affinity value when measured and defined in this way is 0.9, 0.8, 0.7, 0.6, 0.5, 0.4, 0.3, 0.2 or 0.1 μg/ml or less (i.e. the binding agent demonstrates greater than 50% maximal binding at 0.9, 0.8, 0.7, 0.6, 0.5, 0.4, 0.3, 0.2 or 0.1 μg/ml or less when measured in the flow cytometry assay defined above). In representative embodiments, the affinity value when measured in this way is 0.5 or 0.1 μg/ml or less.

In another aspect, the invention provides binding molecules, as novel products per se, which comprise, or are based on or derived from, the variable light (VL) and variable (VH) domains (or regions), or the CDR regions thereof, of the novel antibodies which have been developed in the making of this invention, that is binding molecules based on, or derived from, antibodies 7G12 and 59D04 as described herein.

Accordingly, in this aspect the invention further provides a binding agent (and in particular an isolated specific binding agent) which binds human CLPTM1, wherein said binding agent comprises

(i) a VL region having an amino acid sequence as set forth in SEQ ID NO: 45, or an amino acid sequence having at least 80% sequence identity thereto, and a VH region having an amino acid sequence as set forth in SEQ ID NO: 44, or an amino acid sequence having at least 80% sequence identity thereto; or

(ii) a VL region having an amino acid sequence as set forth in SEQ ID NO: 42, or an amino acid sequence having at least 80% sequence identity thereto, and a VH region having an amino acid sequence as set forth in SEQ ID NO: 41, or an amino acid sequence having at least 80% sequence identity thereto.

In particular embodiments the level of amino acid sequence identity may be at least 85, 86, 87, 88, 89, 90, 91, 92, 93, 94, 95, 96, 97, or 99%. Sequence identity may be determined as described above.

In some embodiments the framework regions of said VL and VH sequences have at least 80% amino acid sequence identity to the framework regions of SEQ ID NOS. 44 or 42 and/or 45 or 41, respectively. For example in some embodiments the framework regions of SEQ ID NOS: 44 and 45 may be humanised.

This aspect of the invention also provides a binding agent (and in particular an isolated specific binding agent) which binds human CLPTM1 and which comprises the complementarity-determining regions (CDRs) VLCDR1, VLCDR2, VLCDR3, VHCDR1, VHCDR2 and VHCDR3, wherein

(i) each of said CDRs has an amino acid sequence as follows:

VLCDR1 has the sequence set forth in SEQ ID NO: 51;

VLCDR2 has the sequence set forth in SEQ ID NO: 52;

VLCDR3 has the sequence set forth in SEQ ID NO: 53;

VHCDR1 has the sequence set forth in SEQ ID NO: 54;

VHCDR2 has the sequence set forth in SEQ ID NO: 55; and

VHCDR3 has the sequence set forth in SEQ ID NO: 56;

or, for each sequence, an amino acid sequence with at least 85% sequence identity thereto,

or wherein one or more of said CDR sequences of SEQ ID NOs: 51 to 56 (or more particularly one more of said CDR1 and CDR2 sequences) may optionally be modified by substitution, addition or deletion of 1 to 3 (e.g. 1 or 2) amino acids; or

(ii) each of said CDRs has an amino acid sequence as follows:

VLCDR1 has the sequence set forth in SEQ ID NO: 57;

VLCDR2 has the sequence set forth in SEQ ID NO: 58;

VLCDR3 has the sequence set forth in SEQ ID NO: 59;

VHCDR1 has the sequence set forth in SEQ ID NO: 60;

VHCDR2 has the sequence set forth in SEQ ID NO: 61; and

VHCDR3 has the sequence set forth in SEQ ID NO: 62;

or, for each sequence, an amino acid sequence with at least 85% sequence identity thereto,

or wherein one or more of said CDR sequences of SEQ ID NOs: 57 to 62 (or more particularly one more of said CDR1 and CDR2 sequences) may optionally be modified by substitution, addition or deletion of 1 to 3 (e.g. 1 or 2) amino acids.

By “or, for each sequence, an amino acid sequence with at least 85% sequence identity thereto” is meant that each of the said CDRs may have the amino acid sequence specified in the relevant SEQ ID NO, or an amino acid sequence with at least 85% sequence identity thereto. Thus, in parts (i) and (ii) respectively, VLCDR1 has the sequence set forth in SEQ ID NO: 51 or 57, or an amino acid sequence with at least 85% sequence identity to SEQ ID NO: 51 or 57; VLCDR2 has the sequence set forth in SEQ ID NO: 52 or 58, or an amino acid sequence with at least 85% sequence identity to SEQ ID NO: 52 or 58; VLCDR3 has the sequence set forth in SEQ ID NO: 53 or 59, or an amino acid sequence with at least 85% sequence identity to SEQ ID NO: 53 or 59; VHCDR1 has the sequence set forth in SEQ ID NO: 54 or 60, or an amino acid sequence with at least 85% sequence identity to SEQ ID NO: 54 or 60; VHCDR2 has the sequence set forth in SEQ ID NO: 55 or 61, or an amino acid sequence with at least 85% sequence identity to SEQ ID NO: 55 or 61; and VHCDR3 has the sequence set forth in SEQ ID NO: 56 or 62, or an amino acid sequence with at least 85% sequence identity to SEQ ID NO: 56 or 62.

In particular embodiments of the invention, in parts (i) and (ii) respectively VLCDR1 has (by which is meant herein consists of) the sequence set forth in SEQ ID NO: 51 or 57, VLCDR2 has the sequence set forth in SEQ ID NO: 52 or 58, VLCDR3 has the sequence set forth in SEQ ID NO: 53 or 59, VHCDR1 has the sequence set forth in SEQ ID NO: 54 or 60, VHCDR2 has the sequence set forth in SEQ ID NO: 55 or 61; and VHCDR3 has the sequence set forth in SEQ ID NO: 56 or 62. The sequences used in the binding molecule may comprise the sequences described herein.

The binding agent of this aspect of the invention may alternatively be defined as comprising a VL sequence and a VH sequence each comprising three CDR sequences, wherein the CDR sequences are as defined above.

In embodiments of this aspect of the invention the sequence identity may be at least 86, 87, 88, 89, 90, 91, 92, 93, 94, 95, 96, 97, or 99%. Sequence identity may be determined as described above.

In some embodiments the amino acid sequences of at least CDR3 of said VL and VH sequences are unmodified, and preferably the amino acid sequences of all of the CDRs are unmodified.

In some embodiments the combined sequences of the CDRs have at least 90% sequence identity to the combined sequences set forth in SEQ ID NOs: 51 to 55, or SEQ ID NOs: 57 to 62, respectively.

By the “combined sequence of the CDR sequences” (or the combined sequences of the CDRs) is meant the sequence formed when the sequences are assembled end-to-end (even if in the molecule of interest they would appear with intervening sequences). In other words, the combined sequence of the CDR sequences is the amino acid sequence obtained when the CDR sequences are joined together in the order listed above (i.e. VLCDR1-VLCDR2-VLCDR3-VHCDR1-VHCDR2-VHCDR3), thus the combined sequence has at its N-terminus the N-terminal amino acid of VLCDR1; the C-terminus of VLCDR1 is joined directly to the N-terminus of VLCDR2; the C-terminus of VLCDR2 is joined directly to the N-terminus of VLCDR3; the C-terminus of VLCDR3 is joined directly to the N-terminus of VHCDR1; the C-terminus of VHCDR2 is joined directly to the N-terminus of VHCDR3; and the C-terminal amino acid of VHCDR3 forms the C-terminus of the combined sequence. By “joined directly” herein is meant that the N-terminal amino acid of a particular CDR sequence is placed immediately next to the C-terminal amino acid of the preceding CDR sequence, with no intervening amino acids (for the purposes of sequence identity assessment).

When a CDR sequence is modified by substitution of a particular amino acid residue, the substitution may be a conservative amino acid substitution. The term “conservative amino acid substitution”, as used herein, refers to an amino acid substitution in which one amino acid residue is replaced with another amino acid residue having a similar side chain. Amino acids with similar side chains tend to have similar properties, and thus a conservative substitution of an amino acid important for the structure or function of a polypeptide may be expected to affect polypeptide structure/function less than a non-conservative amino acid substitution at the same position. Families of amino acid residues having similar side chains have been defined in the art, including basic side chains (e.g. lysine, arginine, histidine), acidic side chains (e.g. aspartic acid, glutamic acid), uncharged polar side chains (e.g. asparagine, glutamine, serine, threonine, tyrosine), non-polar side chains (e.g. glycine, cysteine, alanine, valine, leucine, isoleucine, proline, phenylalanine, methionine, tryptophan) and aromatic side chains (e.g. tyrosine, phenylalanine, tryptophan, histidine). Thus a conservative amino acid substitution may be considered to be a substitution in which a particular amino acid residue is substituted for a different amino acid in the same family. However, a substitution of a CDR residue may equally be a non-conservative substitution, in which one amino acid is substituted for another with a side-chain belonging to a different family.

Amino acid substitutions or additions in the scope of the invention may be made using a proteinogenic amino acid encoded by the genetic code, a proteinogenic amino acid not encoded by the genetic code, or a non-proteinogenic amino acid. Preferably any amino acid substitution or addition is made using a proteinogenic amino acid. The amino acids making up the sequence of the CDRs may include amino acids which do not occur naturally, but which are modifications of amino acids which occur naturally. Providing these non-naturally occurring amino acids do not alter the sequence and do not affect specificity, they may be used to generate CDRs described herein without reducing sequence identity, i.e. are considered to provide an amino acid of the CDR. For example derivatives of the amino acids such as methylated amino acids may be used.

Modifications to the amino acid sequences of the CDRs set out above may be made using any suitable technique, such as site-directed mutagenesis of the encoding DNA sequence or solid state synthesis.

Binding agents of the invention comprise the above described CDRs. Additionally such molecules may contain linker moieties or framework sequences to allow appropriate presentation of the CDRs. Suitable linker molecules are well known in the art. Additional sequences may also be present which may conveniently confer additional properties, e.g. peptide sequences which allow isolation or identification of the molecules containing the CDRs such as those described hereinbefore. In such cases a fusion protein may be generated.

The binding agent of these aspects of the invention may be an antibody, as defined and described above and herein. In particular it may be it may be an antibody fragment, e.g. a Fab or F(ab′)₂ or Fv antibody fragment, or a single chain antibody molecule, e.g. a scFv molecule. In the case of binding agents based on antibody 7G12 (i.e. as defined in parts (i) above), in certain embodiments the binding agent may be a chimeric, or humanised, antibody. A humanised antibody is an antibody derived from another species, e.g. a mouse, in which not only are the constant domains of the antibody chains replaced with human constant domains, but the amino acid sequences of the variable regions are modified, in particular to replace the foreign (e.g. murine) framework sequences with human framework sequences, such that, preferably, the only non-human sequences in the antibody are the CDR sequences.

In an antibody, the CDR sequences are located in the variable domains of the heavy and light chains. The CDR sequences sit within a polypeptide framework, which positions the CDRs appropriately for antigen binding. Thus the remainder of the variable domains (i.e. the parts of the variable domain sequences which do not form a part of any one of the CDRs) constitute framework regions. The N-terminus of a mature variable domain forms framework region 1 (FR1); the polypeptide sequence between CDR1 and CDR2 forms FR2; the polypeptide sequence between CDR2 and CDR3 forms FR3; and the polypeptide sequence linking CDR3 to the constant domain forms FR4. In an antibody of the invention the variable region framework regions may have any appropriate amino acid sequence such that the antibody binds to CLPTM1 (or more particularly the ECD thereof) via its CDRs. The constant regions may be the constant regions of any mammalian (preferably human) antibody isotype.

The binding agent may further have any one or more of the properties, e.g. activities or functional properties, described herein for binding agents for use according to the present invention. For example the binding agent may have an affinity as defined and described herein. The binding agent may additionally or alternatively have any one or more of the immunosuppressive activities described or defined herein, e.g. the binding agent may be capable of promoting or inducing an anti-inflammatory secretome (or environment) or cytokine or reducing or reversing a pro-inflammatory secretome (or environment) or cytokine, and/or it may be capable of down-regulating STING, and/or it may be capable of internalising CLPTM1.

The invention also provides pharmaceutical composition comprising a binding agent of the invention as defined above together with at least one pharmaceutically acceptable carrier, diluent or excipient, and a binding agent of the invention as defined above for use in therapy.

The binding agent may be synthesised by any method known in the art. In particular, the binding agent may be synthesised using a protein expression system, such as a cellular expression system using prokaryotic (e.g. bacterial) cells or eukaryotic (e.g. yeast, fungus, insect or mammalian) cells. Cells which may be used in the production of the specific binding molecule are discussed further below. An alternative protein expression system is a cell-free, in vitro expression system, in which a nucleotide sequence encoding the binding agent is transcribed into mRNA, and the mRNA translated into a protein, in vitro. Cell-free expression system kits are widely available, and can be purchased from e.g. ThermoFisher Scientific (USA). Alternatively, binding agents may be chemically synthesised in a non-biological system. Liquid-phase synthesis or solid-phase synthesis may be used to generate polypeptides which may form or be comprised within the binding agent of the invention. The skilled person can readily produce binding agents using appropriate methodology common in the art.

In further aspects the invention provides a nucleic acid molecule having or comprising a nucleotide sequence encoding the binding agent of the invention as defined herein.

In one embodiment such a nucleic acid may have or may comprise a nucleotide sequence as set forth in SEQ ID NO: 46 or 47, or a nucleotide sequence having at least 80% sequence identity thereto.

Also provided are recombinant constructs and vectors comprising a nucleic acid molecule of the invention. In the construct, the nucleic acid molecule of the invention may be flanked by restriction sites (i.e. nucleotide sequences recognised by one or more restriction enzymes) to enable easy cloning of the nucleic acid molecule of the invention. In the construct of the invention the nucleotide sequence encoding the specific binding molecule of the invention may conveniently be operably linked within said construct to an expression control sequence, which may be heterologous to the nucleic acid molecule, i.e. non-native. Such an expression control sequence is typically a promoter, though the nucleotide sequence encoding the specific binding molecule may alternatively or additionally be operably linked to other expression control sequences such as a terminator sequence, an operator sequence, an enhancer sequence or suchlike. Accordingly, the construct may comprise a native or non-native promoter. The term “vector” as used herein refers to a vehicle into which the nucleic acid molecule or construct of the invention may be introduced (e.g. be covalently inserted) from which the specific binding molecule or mRNA encoding it may be expressed and/or the nucleic acid molecule/construct of the invention may be cloned. The vector may accordingly be a cloning vector or an expression vector.

The invention also provides host cells comprising a nucleic acid molecule, construct or vector of the invention. It will be understood that the host cell does not include native cells i.e. which contain the nucleic acid molecule encoding the binding agent as a native or endogenous molecule. Thus, the nucleic acid molecule is heterologous to the cell, or has been introduced into the cell. The cell may thus be termed a transgenic host cell.

The host cell may be a prokaryotic (e.g. bacterial) or eukaryotic (e.g. mammalian) cell. A prokaryotic cell may in particular be used as a cloning host for the nucleic acid molecule, construct or vector of the invention. Suitable prokaryotic cells for use as cloning hosts include without limitation, eubacteria, such as Gram-negative or Gram-positive organisms, for example, Enterobacteriaceae such as Escherichia, in particular E. coli, and Bacilli such as B. subtilis. The cloning host may alternatively be a eukaryotic cell such as a fungal cell, e.g. Pichia pastoris, or a yeast cell, or a higher eukaryotic cell such as a mammalian cell.

The host cell of the invention may alternatively be a production host, i.e. a cell used to express and produce the binding agent of the invention. The production host cell may be a prokaryotic cell, as defined above, but is preferably a eukaryotic cell, for example a mammalian cell, particularly a rodent cell, a human cell or a cell of an alternative primate.

Particular examples of cells which may constitute a production host according to the invention include Cos cells, such as COS-7 cells, HEK293 cells, CHO cells, though any suitable cell type or line may be used.

Further provided is a method of preparing a binding agent of the invention as defined herein, said method comprising:

i) introducing into a host cell a nucleic acid molecule, a construct or a vector as defined herein;

ii) expressing the nucleic acid molecule such that the binding agent is produced; and

iii) collecting the binding agent, e.g. isolating, separating or purifying the binding agent.

A composition or a combination product (e.g. a kit) may be provided which comprises a natural ligand for CPTM1 (e.g. GDF15 or a TGF-β protein, such as TGF-β1, 2 or 3) in combination with another binding agent which is not a natural ligand for CLPTM1 (e.g. an antibody).

Although the mechanism of action of the binding agents at the CLPTM1 receptor to induce the observed effects is not yet clear, one possible theory is that the observed effects may be induced by receptor dimerisation. Without wishing to be bound by theory, it is hypothesised that a binding agent which is capable of simultaneously binding to two or more separate CLPTM1 molecules may induce dimerisation of CLPTM1; the known endogenous ligands are homodimers and thus binding agents such as antibodies may be acting through two contact points, analogously to the endogenous ligands. Thus, in a preferred embodiment of the present invention, the binding agent may comprise two or more separate binding sites for CLPTM1 According to such an embodiment, the binding agent may be an antibody or an Fc-fusion protein. In such an embodiment where an antibody is used it is bivalent, and accordingly where the antibody is an antibody fragment or modified antibody or derivative it is bivalent.

As noted above, the binding agents of the present invention may be provided in the form of a composition, in particular in the form a pharmaceutical composition which may be used or may be provided for use in treating or preventing a condition described above. Such compositions may comprise or contain a binding agent according to the present invention in combination with one or more pharmaceutically acceptable diluent, carrier or excipient. “Pharmaceutically acceptable” as referred to herein refers to ingredients that are compatible with other ingredients of the compositions as well as physiologically acceptable to the recipient. The nature of the composition and carriers or excipient materials, dosages etc. may be selected in routine manner according to choice and the desired route of administration, pH, temperature etc.

Thus, the binding agent may be incorporated optionally together with other active substances as a combined preparation, with one or more conventional carriers, diluents and/or excipients, to produce conventional galenic preparations such as powders, sachets, suspensions, emulsions, solutions, aerosols (as a solid or in a liquid medium), ointments, and the like. The compositions may be stabilized by use of freeze-drying, undercooling or Permazyme.

Suitable excipients, carriers or diluents are lactose, dextrose, sucrose, sorbitol, mannitol, starches, gum acacia, calcium phosphate, calcium carbonate, calcium lactose, corn starch, alginates, tragacanth, gelatin, calcium silicate, microcrystalline cellulose, polyvinylpyrrolidone, cellulose, water syrup, water, water/ethanol, water/glycol, water/polyethylene, glycol, propylene glycol, methyl cellulose, methylhydroxybenzoates, propyl hydroxybenzoates, talc, magnesium stearate, mineral oil or fatty substances such as hard fat or suitable mixtures thereof.

Agents for obtaining sustained release formulations, such as carboxypolymethylene, carboxymethyl cellulose, cellulose acetate phthalate, or polyvinylacetate may also be used. The compositions may additionally include lubricating agents, wetting agents, emulsifying agents, viscosity increasing agents, granulating agents, disintegrating agents, binding agents, osmotic active agents, suspending agents, preserving agents, adsorption enhancers, organic solvent, antioxidant, stabilizing agents, emollients, silicone, alpha-hydroxy acid, demulcent, anti-foaming agent, moisturizing agent, vitamin, ionic or non-ionic thickeners, surfactants, filler, ionic or non-ionic thickener, sequestrant, polymer, propellant, alkalinizing or acidifying agent, opacifier, colouring agents and fatty compounds and the like.

The compositions of the invention may be formulated so as to provide quick, sustained or delayed release of the binding agent after administration to a patient by employing techniques well known in the art.

The use of solutions, suspensions, gels and emulsions (or powders which may be made into such forms) are preferred, e.g. the active ingredient may be carried in water, a water-based liquid, an oil, a gel, an emulsion, an oil-in water or water-in-oil emulsion, a dispersion or a mixture thereof.

Topical compositions and administration are use which include gels, creams, ointments, sprays, lotions, salves, sticks, soaps, powders, films, aerosols, drops, foams, solutions, emulsions, suspensions, dispersions e.g. non-ionic vesicle dispersions, milks and any other conventional pharmaceutical forms in the art. Ointments, gels and creams may, for example, be formulated with an aqueous or oily base with the addition of suitable thickening and/or gelling agents. Lotions may be formulated with an aqueous or oily base and will, in general, also contain one or more emulsifying, dispersing, suspending, thickening or colouring agents. Powders may be formed with the aid of any suitable powder base. Drops and solutions may be formulated with an aqueous or non-aqueous base also comprising one or more dispersing, solubilising or suspending agents. Aerosol sprays are conveniently delivered from pressurised packs, with the use of a suitable propellant.

Likewise the binding agent may be administered in any convenient or desired manner, e.g. parenterally or non-parenterally, for example by enteral administration, e.g. orally (depending on the nature and/or formulation of the agent), or by intravenous, sub-cutaneous, intramuscular, intraperitoneal injection or infusion. The administration may be systemic or local, depending e.g. on the condition to be treated, the nature of the agent and/or formulation etc. Thus, for example the binding agent may be delivered locally e.g. by infusion or direct injection, e.g. to the site or location of a cancer.

In addition to the pharmaceutically acceptable carrier or excipient, the composition may comprise at least 0.0005% of the binding agent (w/w) as a percentage of its total composition. Thus, the composition (at the point of administration) may comprise at least 0.0005, 0.001 or 0.005 to 25%, e.g. 0.005 to 1% or 0.001 to 10%, such as 0.005 to 0.5% of the binding agent (w/w of the final preparation for administration, particularly for topical administration).

Dosages of the binding agent may also be determined in routine manner according to standard clinical practice. Doses of 0.1-10 mg/kg, such as doses of 0.1-0.5 mg/kg, 0.1-1 mg/kg, 0.1-2 mg/kg, 0.1-5 mg/kg, 0.5-1 mg/kg, 0.5-2 mg/kg, 0.5-5 mg/kg, 0.5-10 mg/kg, 1-2 mg/kg, 1-5 mg/kg, 1-10 mg/kg, 2-5 mg/kg, 2-mg/kg or 5-10 mg/kg may be administered daily, weekly, or every 10 days, 2 weeks, 3 weeks or monthly until disease progression, or until unacceptable toxicity is observed.

Likewise the therapeutic agents may be administered in any convenient or desired manner, e.g. parenterally or non-parenterally, for example by enteral administration, e.g. orally (depending on the nature and/or formulation of the agent), or by intravenous, sub-cutaneous, intramuscular, intraperitoneal injection or infusion. The administration may be systemic or local, depending e.g. on the condition to be treated, the nature of the agent and/or formulation etc. Thus, for example the agent may be delivered locally e.g. by infusion or direct injection, e.g. to the site or location of a cancer.

The subject may be any human or non-human animal subject, but preferably is a mammalian subject. Although human subjects represent a preferred embodiment, the subject may be any domestic, livestock, laboratory, sport, or zoo animal etc., for example a primate or rodent, or cow, sheep, horse, dog, rabbit etc.

The present invention may be better understood through the following Examples and Figures, in which:

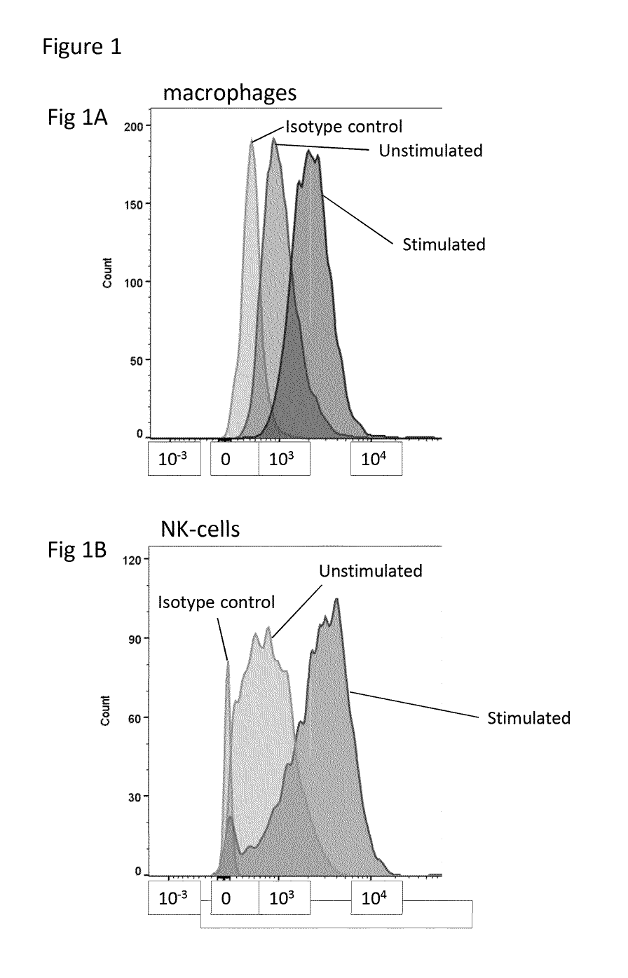

FIG. 1 shows that surface expression of CLPTM1 is increased in different types of immune cell in response to stimulation with LPS for macrophages and CD4/CD28 beads for NK-cells. FIG. 1A—surface expression of CLPTM1 in CD14+ macrophages is increased following stimulation. FIG. 1B—surface expression of CLPTM1 in CD45+ cells is increased following stimulation.

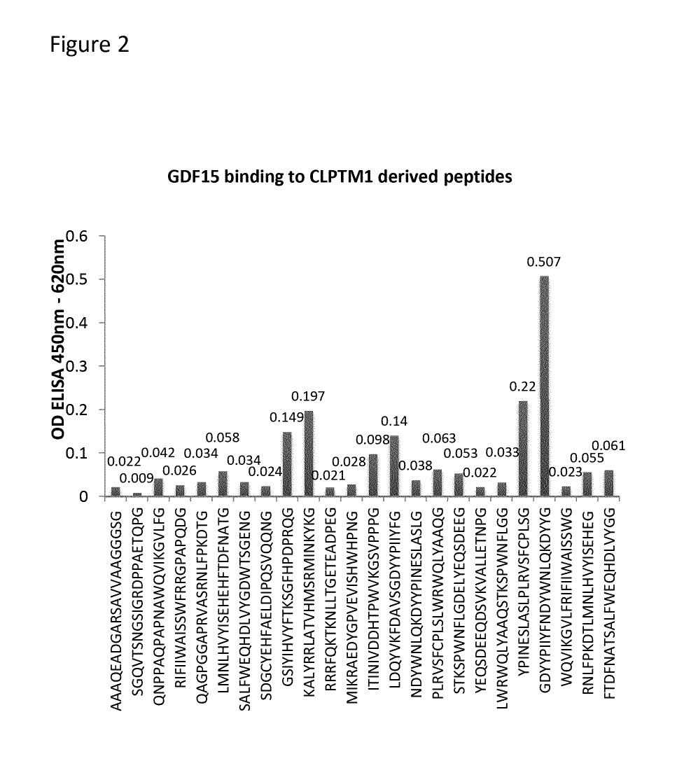

FIG. 2 shows the binding of GDF15 to CLPTM1 derived peptides. Polypeptides having amino acids as set forth in SEQ ID NOs: 10-33 were immobilised in streptavidin-coated well and binding of GDF15 to each peptide was measured.

FIG. 3 shows peptide array data identifying the epitope of the AbCam anti-CLPTM1 mAb tested in various assays in the present application. A core binding motif of DPPA, corresponding to amino acids 34-37 of CLPTM1 was identified. This binding site is situated away from the binding sites for GDF15.

FIG. 4 shows that contacting cells expressing CLPTM1 with GDF15 leads to co-localisation of TMEM173/STING and CLPTM1. FIG. 4A—in the absence of GDF15, only a limited amount of the complex is detectable. FIG. 4B—addition of GDF15 (2 μg/ml for 1 hour) resulted in an increase in the amount of the complex, indicating that TMEM173/STING activity is reduced by GDF15 binding CLPTM1.

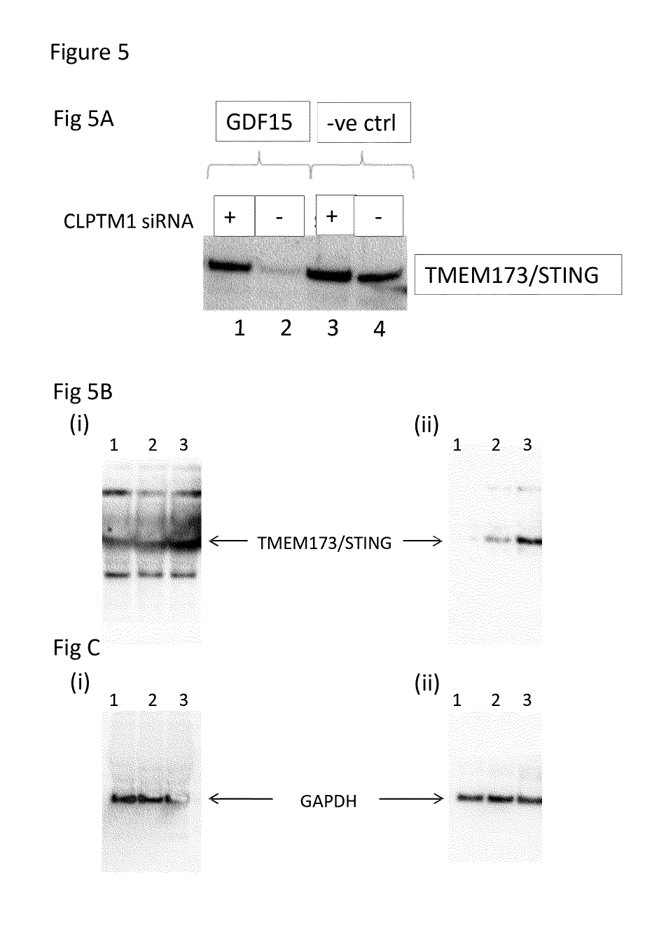

FIG. 5 shows GDF15 and TGB-β3 can induce CLPTM1 degradation. FIG. 5A—CLPTM1 siRNA can reduce GDF15 mediated degradation of TMEM173/STING in MCF7 cells. TMEM173/STING is detected following exposure of cells to CLPTM1 or control siRNA, and GDF15 or a negative control Lane 1: CLPTM1 siRNA and GDF15; Lane 2: control siRNA and GDF15; Lane 3: CLPTM1 siRNA and −ve control; Lane 4: control siRNA and −ve control. In lane 1 CLPTM1 siRNA down-regulates the level of CLPTM1 and prevents GDF15 from binding? CLPTM1 and inducing TMEM173/STING degradation, but in lane 2 where CLPTM1 is still present, GDF15 binds CLPTM1 and induces TMEM173/STING degradation. FIG. 5B—TMEM173/STING degradation can be induced by GDF15 and TGF-β3. The effect of GDF15 or TGF-β3 on CLPTM1 levels in NK92 cells (FIG. 5B (i)) and MCF7 cells (FIG. 5B (ii)) was assessed by Western Blot. Lane 1—cells treated with 10 ng/ml TGF-β3 for 24 hours. Lane 2—cells treated with 2 μg/ml GDF15 for 24 hours. Lane 3—untreated cells. GDF15 and TGF-β3 were both found to cause TMEM173/STING degradation, relative to the control. Following TMEM173/STING detection, the Western Blot membrane was stripped and probed for GAPDH for a loading control (FIG. 5C).

FIG. 6 shows GDF15 and TGB-β3 can induce degradation of PTP1B. The effect of GDF15 or TGF-β3 on PTP1B levels in MCF-7 cells was assessed by detecting PTP1B in whole cell lysates by Western Blot. Lane 1—MCF 7 cells treated with 10 ng/ml TGF-β3 o/n. Lane 2—MCF 7 cells treated with 2 μg/ml GDF15 o/n. Lane 3—untreated MCF7 cells. GDF15 and TGF-β3 were both found to cause PTP1B degradation, relative to the control. Following PTP1B detection, the Western Blot membrane was stripped and probed for GAPDH for a loading control.

FIG. 7 shows that both GDF15 and anti-CLPTM1 antibodies can induce degradation of TMEM173/STING. Cells stimulated with LCP in lanes 2, 4, 6 and 7. Lanes 1-2—negative control. Lanes 3-4—AbCam anti-CLPTM1 antibody. Lanes 5-7—GDF15. Lane 7—3A10 anti-CLPTM1 antibody. The AbCam and 3A10 antibodies and GDF15 all reduce the level of TMEM173/STING compared to the negative control in the presence of LPS. The combination of the 3A10 anti-CLPTM1 antibody and GDF15 leads to a larger decrease than GDF15 alone.

FIG. 8 shows GDF15, TGF-β3 and the AbCam anti-CLPTM1 mAb are capable if inducing internalisation of CLPTM1 into Rab11 endosomes. Rab11-CLPTM1 co-localisation is detected using in situ PLA-RCA (Duolink—Olink Bioscience AB). FIG. 8A—control, no stimulation. FIG. 8B—GDF15 (2 μg/ml for 30 minutes. FIG. 8C—TGF-β3 (10 ng/ml for 30 minutes). FIG. 8D—Quantitation of the degree of Rab11-CLPTM1 co-localisation detected—the AbCam anti-CLPTM1 mAb induces receptor internalisation.

FIG. 9 shows changes in cytokine expression by PBMC culture caused by the AbCam anti-CLPTM1 mAb.

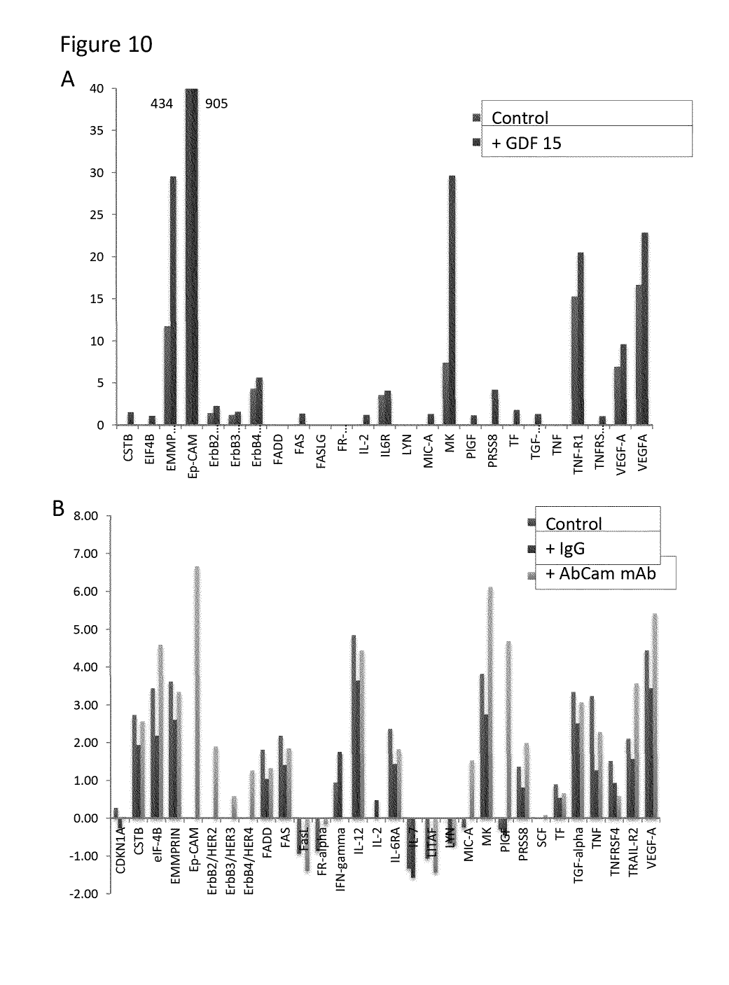

FIG. 10 shows that the AbCam anti-CLPTM1 mAb induces similar changes in the secretion profile of cytokines by PBMC as GDF15. In particular, cytokines that are dis-regulated in Multiple Sclerosis are modulated by both reagents. FIG. 10A—MCF7 cells contacted with GDF15-positive exosomes (data values for Ep-CAM are shown). Expression levels for a negative control are also shown. FIG. 10B—PBMC cells contacted with the AbCam anti-CLPTM1 mAb. Expression levels for a negative control and IgG control are also shown.

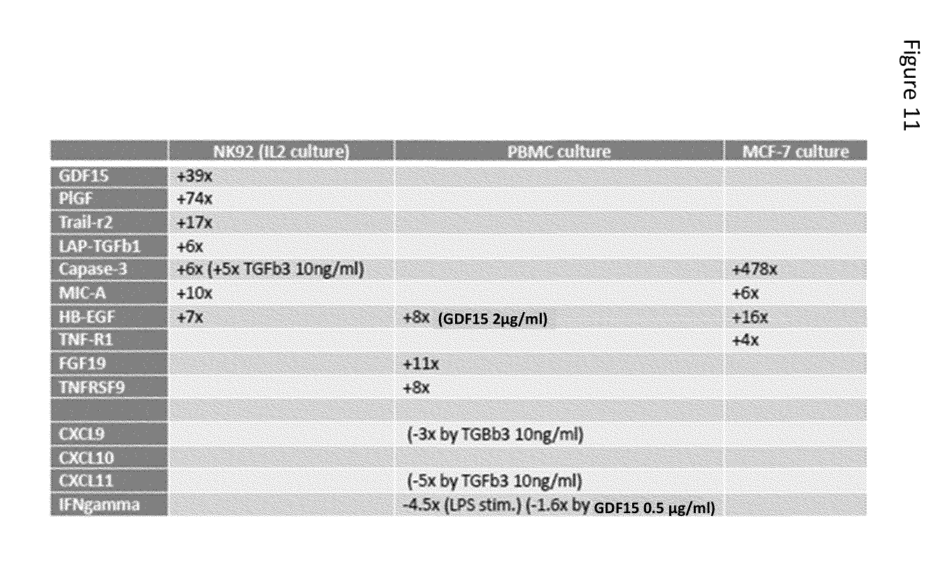

FIG. 11 shows changes in the secretion of certain proteins by NK92, PBMC and MCF7 cells induced by the AbCam anti-CLPTM1 mAb, and GDF and TGF-β3. Data is the fold induction over baseline (control) observed on addition of the AbCam anti-CLPTM1 mAb at 2 μg/ml.

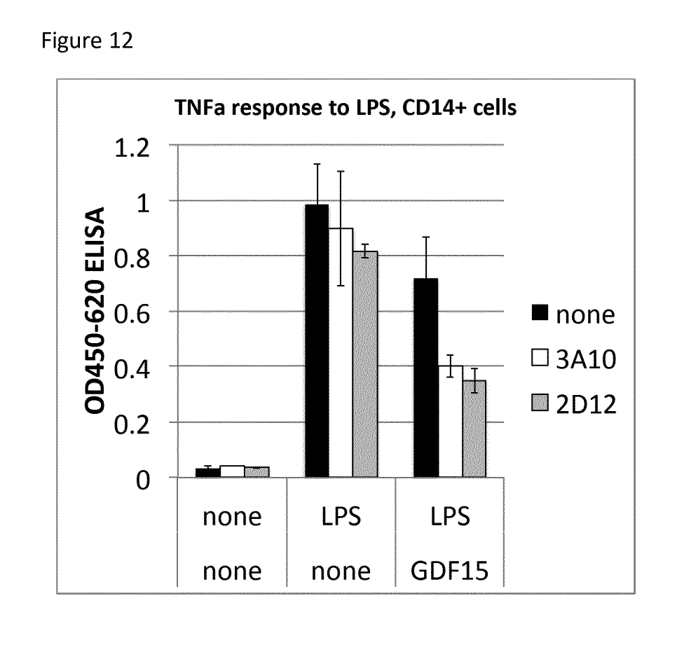

FIG. 12 shows the effect of mouse monoclonal antibodies on the secretion of TNFα in CD14+ cells stimulated with LPS. In the absence of LPS secretion of TNFα is low. Cells contacted with LPS show higher levels of TNFα secretion, but cells pre-incubated with either the 3A10 or 2D12 mouse mAb show lower levels of secretion than cells in the absence of a CLPTM1 binder. Cells pre-incubated with GDF15 and either the 3A10 or 2D12 mouse mAb show substantially lower levels of TNFα secretion compared to cells in the absence of a CLPTM1 agonist, or the antibodies alone.

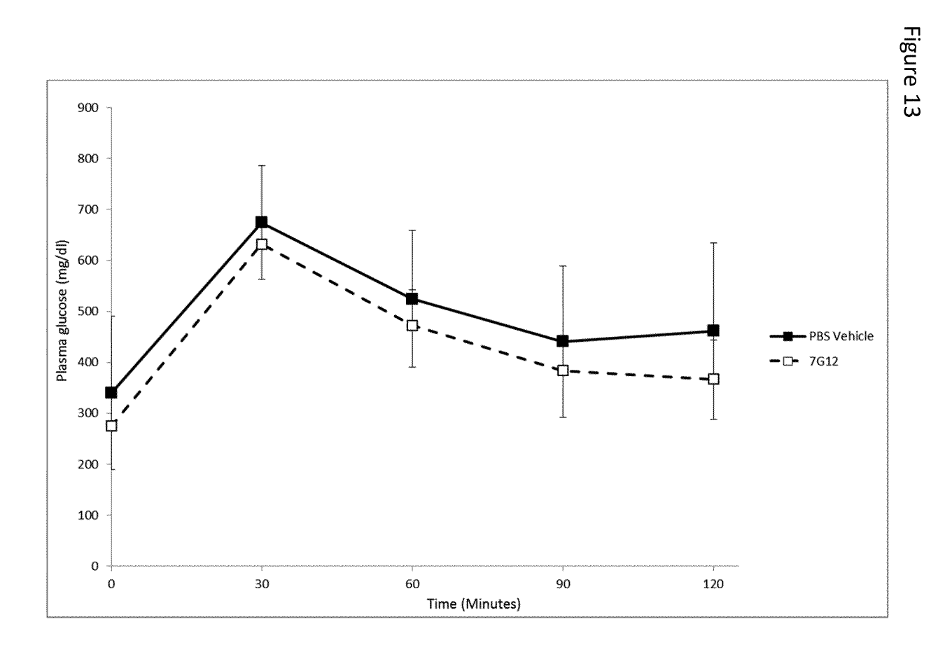

FIG. 13 shows the effect if the 7G12 antibody on glucose tolerance in a mouse model. Glucose response curves for ob/ob mice administered a PBS negative control (solid line) or the 7G12 antibody (dashed line) following administration of 2 g/kg glucose. Mice administered the 7G12 antibody had lower plasma glucose values (mg/dl).

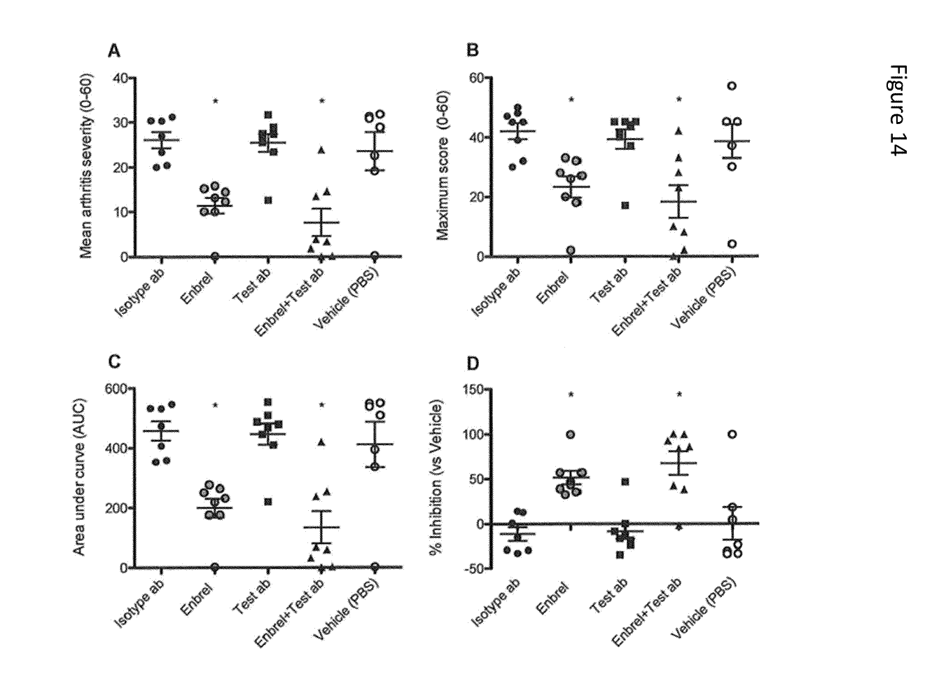

FIG. 14 shows the effect of the 7G12 antibody alone and in combination with a TNF blocker on the severity of disease in an arthritis mouse model. Mean disease severity score (A); maximum severity score (B); area under curve (C) and % inhibition relative to a PBS negative control (D) for a negative control antibody, Enbrel; 7G12 antibody, and combination 7G12+Enbrel.

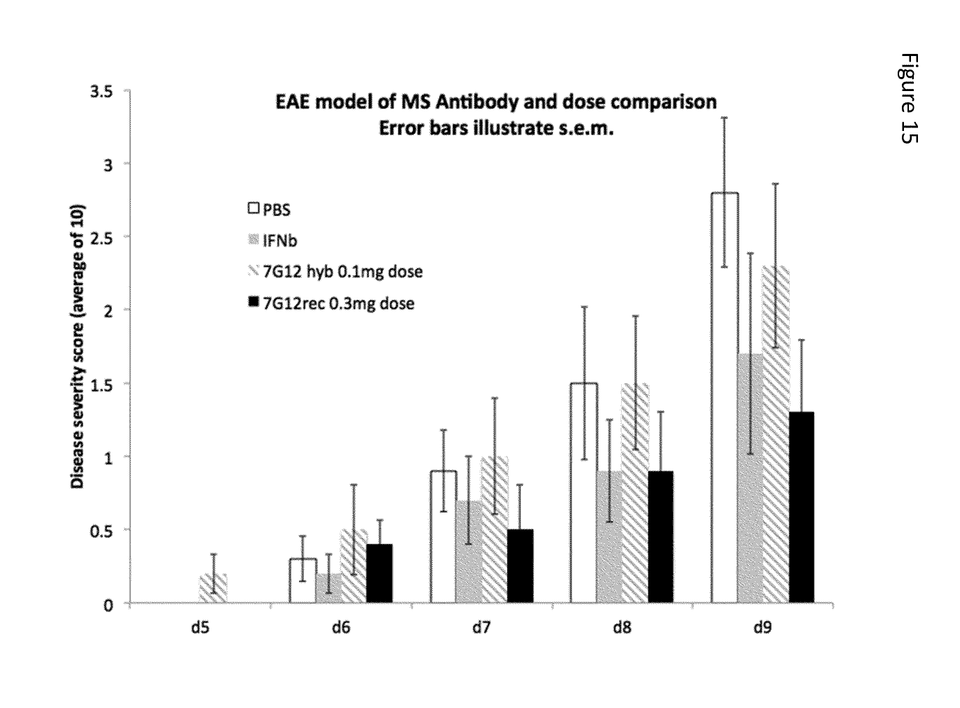

FIG. 15 shows the effect of the 7G12 antibody on the severity of disease in an MS mouse model. Disease severity is shown for a negative control (white); IFNβ (grey), 7G12 hybridoma extract 0.1 mg dose (hatched); and recombinant 7G12 antibody 0.3 mg dose (black). On day 9 the disease severity was significantly lower for the 0.3 mg dose than the negative control.

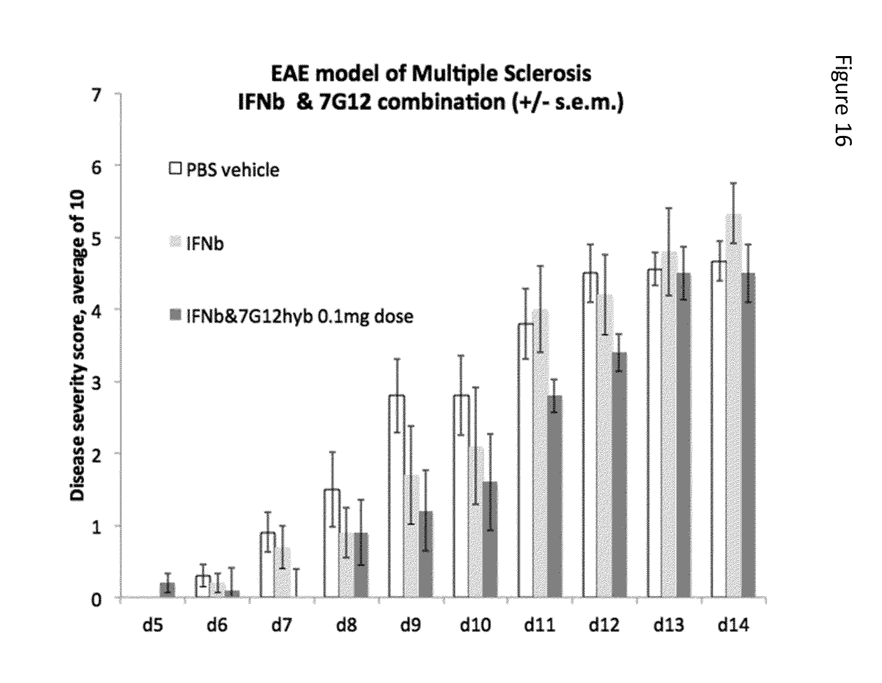

FIG. 16 shows the effect of the 7G12 antibody on the severity of disease in an MS mouse model in a combination therapy with IFNβ. Disease severity is shown for a negative control (white); IGNβ alone (light grey) and combination therapy (dark grey). On days 7 and 9 the disease severity was significantly lower for the combination therapy than the negative control.

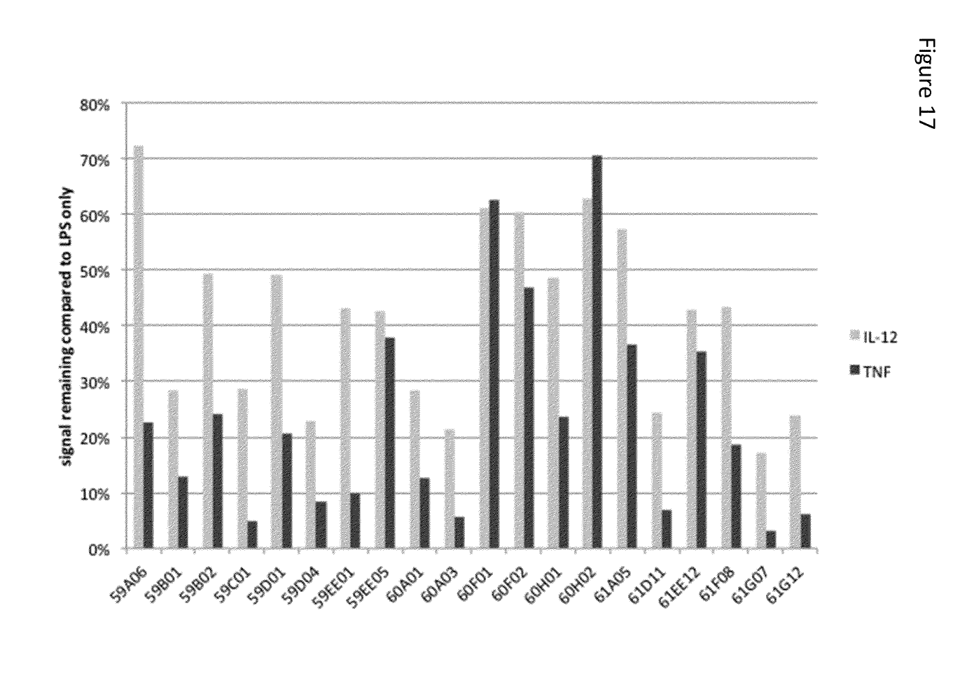

FIG. 17 shows the effect of human antibodies on cytokine secretion by CD14+ cells stimulated with LPS.

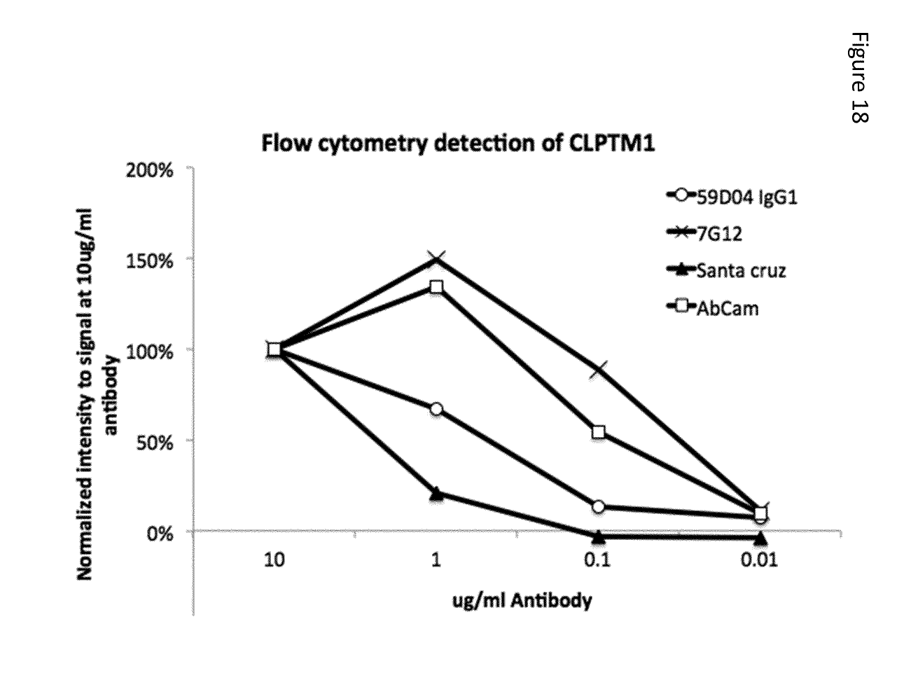

FIG. 18 shows the measurement of the affinity of the 59D04, 7G12, Santa Cruz and AbCam antibodies by flow cytometry.CLPTM1 binding in permeabilised 0-876 cells expressing native CLPTM1 was measured in a 10× dilution series. The highest affinity antibody measured was 7G12, which retained greater than 50% of its maximum binding at 0.1 μg/ml. The AbCam antibody also retained greater than 50% of its maximum binding at 0.1 μg/ml. The 59D04 antibody retained greater than 50% of its maximum binding at 1 μg/ml, whilst the Santa Cruz antibody had less than 50% of its maximum binding at this level.

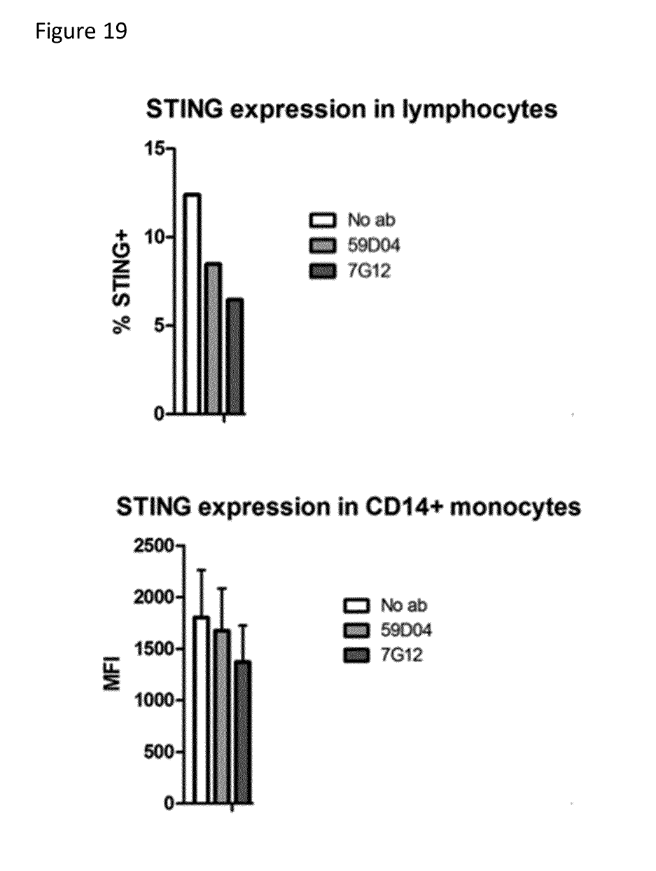

FIG. 19 shows the effect of the 59D04 and 7G12 antibodies on STING expression in lymphocytes. (A) the % of cells having detectable STING expression was reduced when cells were contacted with either antibody. (B) Once CD14+ cells were gated, the mean fluorescence intensity for CD14+ cells contacted with either antibody was reduced.

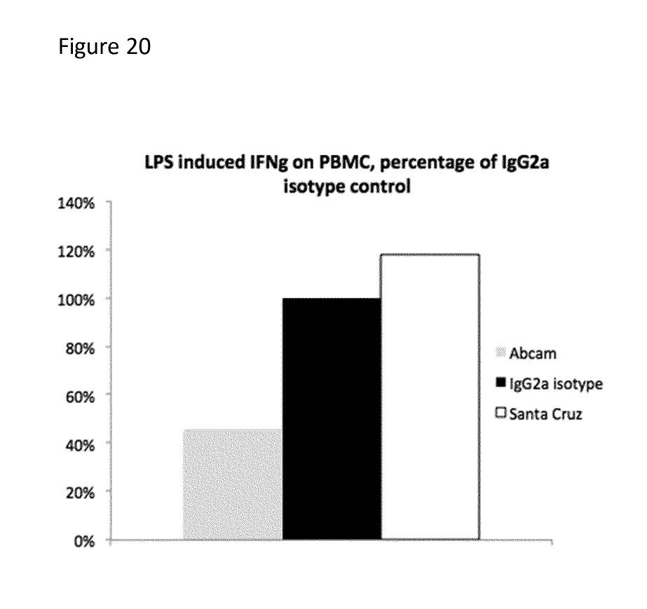

FIG. 20 shows the effect of Santa Cruz and Abcam antibodies on the secretion of IFNγ induce by LPS in PBMC. The figure shows percentage of signal of IFNγ detection in comparison to isotype control antibody (IgG2A antibody).

### EXAMPLES

### Example 1—CLPTM1 Cell Surface Expression in Immune Cells

### Materials and Methods

Cells were stimulated with LPS (macrophages) or CD4/CD28 beads (NK-cells), or a negative control (no stimulation). Cells were counted and used at 1×10⁶ cells/FACS tube. Cells were centrifuged at 300×g for 5 minutes and the supernatant discarded. The cell pellet was resuspended in 500 μl live/dead viability dye blue (diluted 1:1000 in PBS) and incubated for Incubated at 4° C. for 30 min. Following incubation, cells were washed with 2 ml PBS, and pellets were resuspended in 250 μl 10% HUS and incubated for 5 min at room temperature. An Fc-blocking agent (Miltenyi) was added at 20 μl/well. 50 μl antibody cocktail was added to each tube and incubated 30 min at 4° C. 2 ml FACS buffer was added to each tube, and the cells were centrifuged at 300×g for 5 min. Cell pellets were resuspended in PBS and analysed in Fortessa FACS. Compensation for corresponding fluorophores of antibodies was applied. Gating for CD14+ macrophages or CD45RO+ NK cells was applied.

Antibodies used included CD4(Pacific blue) (558116,BD); CLPTM1 (unconjugated at 1 μg/well) Bioss(8018R), secondary A488; CD45(V500); CD14 (PE-Dazzle594) (325634, Biolegend); CD11c (PerCp-Cy5.5) (337210, Biolegend); CD3(A700) (557943,BD); CD56(APC) (555518,BD); CD45RO (APC-Cy7); Isotype control (PE-Cy5) in isotype control panel. Viability dye Blue.

### Results

Both macrophages and NK cells exhibit elevated levels of CLPTM1 on their cell surface, as detectable by flow cytometry, in response to stimulation with an immune stimulating agent (compare “stimulated” with “unstimulated” profiles in FIGS. 1A and 1B).

### Example 2—Identifying the GDF15 Binding Site in CLPTM1

### Materials and Methods

Biotinylated synthetic peptide fragments from CLPTM1 (JPT peptides, Germany) were immobilized in a streptavidin 96 well plate (#15500 Pierce) at 1 μM in PBS overnight at +4° C. Plate was washed 4 times in 300 μl PBS with 0.05% Tween-20 (wash buffer). The plate was blocked with PBS 1% BSA and 0.05% Tween-20 (blocking buffer) for 1.5 hours at room temperature then washed 4 times in wash buffer. GDF15 ligand (Abcam) was added in blocking buffer at 100 ng/ml and incubated at room temperature for 2 hours. The plate was then washed 8 times in 300 μl wash buffer with 2×NaCl. A GDF15 antibody as added at 1 μg/ml (R&D systems goat polyclonal) and incubated in block buffer for 1 hour at room temperature followed by washing with 4×300 μl wash buffer. HAF017 anti-goat-HRP antibody was added and incubated for 1 hour in blocking buffer followed by a wash. TMB substrate was added and stopped after 15 minutes with H2SO4. OD 450-620 was measured in an ELISA reader.

Polypeptides having sequences set forth in SEQ ID NOs:10-33 were assessed for binding to GDF15 (corresponding to the polypeptides indicated from left to right in FIG. 2 and shown in Table 1). These proteins were synthesised with a C-terminal glycine residue that is not present in the corresponding sequences from CLPTM1. The signal measured for each polypeptide is shown in FIG. 2.

### Results

A cut-off of approximately greater than or equal to 0.1 OD in the ELISA readout was identified as indicating binding of the GDF15 to an immobilised polypeptide (i.e. including the 0.098 OD value for GDF15 binding to the polypeptide having the sequence set forth in SEQ ID NO:22). The following peptides showed binding to GDF15:

### (Residue Numbers Relative to SEQ ID NO:1):

(SEQ ID NO: 18)
GSIYIHVYFTKSGFHPDPRQG (162-181)
(SEQ ID NO: 19)
KALYRRLATVHMSRMINKYKG (182-201)
(SEQ ID NO: 22)
ITINIVDDHTPWVKGSVPPPG (242-261)
(SEQ ID NO: 23)
LDQYVKFDAVSGDYYPIIYFG (262-281)
(SEQ ID NO: 29)
YPINESLASLPLRVSFCPLSG (292-311)
SEQ ID NO: 30)
GDYYPIIYFNDYWNLQKDYYG (273-292)

The corresponding polypeptides to SEQ ID NO:18, 19, 22, 23, 29 and 30 lacking the C-terminal glycine residue are 34, 35, 36, 37, 38 and 39, respectively. The binding agents of the present invention may bind to any one of these polypeptides, or parts thereof, as indicated above.

TABLE 1
Peptides used in Example 2 to map GDF15 binding to
CLPTM1. All amino acid residue numbers are
relative to SEQ ID NO: 1

| Peptide | | Amino acid | SEQ |
|---|---|---|---|
| name | Peptide sequence | residues | ID NO: |
| G1 | AAAQEADGARSAVVAAGGGSG | 2-21 | 10 |
| G2 | SGQVTSNGSIGRDPPAETQPG | 22-41 | 11 |
| G3 | QNPPAQPAPNAWQVIKGVLFG | 42-61 | 12 |
| G4 | RIFIIWAISSWFRRGPAPQDG | 62-81 | 13 |
| G5 | QAGPGGAPRVASRNLFPKDTG | 82-101 | 14 |
| G6 | LMNLHVYISEHEHFTDFNATG | 102-121 | 15 |
| G7 | SALFWEQHDLVYGDWTSGENG | 122-141 | 16 |
| G8 | SDGCYEHFAELDIPQSVQQNG | 142-161 | 17 |
| G9 | GSIYIHVYFTKSGFHPDPRQG | 162-181 | 18 |
| G10 | KALYRRLATVHMSRMINKYKG | 182-201 | 19 |
| G11 | RRRFQKTKNLLTGETEADPEG | 202-221 | 20 |
| G12 | MIKRAEDYGPVEVISHWHPNG | 222-241 | 21 |
| G13 | ITINIVDDHTPWVKGSVPPPG | 242-261 | 22 |
| G14 | LDQYVKFDAVSGDYYPIIYFG | 262-281 | 23 |
| G15 | NDYWNLQKDYYPINESLASLG | 282-301 | 24 |
| G16 | PLRVSFCPLSLWRWQLYAAQG | 302-321 | 25 |
| G17 | STKSPWNFLGDELYEQSDEEG | 322-342 | 26 |
| G18 | YEQSDEEQDSVKVALLETNPG | 335-374 | 27 |
| G19 | LWRWQLYAAQSTKSPWNFLGG | 312-331 | 28 |
| G20 | YPINESLASLPLRVSFCPLSG | 292-311 | 29 |
| G21 | GDYYPIIYFNDYWNLQKDYYG | 273-292 | 30 |
| G22 | WQVIKGVLFRIFIIWAISSWG | 53-72 | 31 |
| G23 | RNLFPKDTLMNLHVYISEHEG | 94-113 | 32 |
| G24 | FTDFNATSALFWEQHDLVYGG | 115-134 | 33 |

### Example 3—Epitope Mapping of AbCam Anti-CLPTM1 mAb

### Materials and Methods

The N-terminal part of the extra-cellular domain of CLPTM1 was divided into 86 unique 15-mer peptides with 11-mer overlap and immobilized on glass (array purchased from JPT technologies) and incubated with ligands or antibodies in individual wells. See Table 2 for certain peptide sequences. After extensive washes in PBS-T, bound primary antibodies were detected using goat anti-rabbit (life tech) at 1:60000 and analysed on a G2502 microarray scanner (Agilent technologies).

### Results

The AbCam anti-CLPTM1 (rabbit monoclonal EPR8800; RabMab®) was found to bind well to peptides 6, 7 and 8 (FIG. 3). These peptides share a common core motif (DPPA) as set forth in SEQ ID NO:3, which corresponds to amino acids 34-37 of SEQ ID NO:1 (full length CLPTM1). This position is towards the N-terminal of the receptor, and notably does not overlap with the binding sites for GDF15 which are identified above in Example 2. This suggests that a binding agent of the present invention may be able to bind to portions of the CLPTM1 receptor which are distinct from the binding sites of the natural ligands for CLPTM1, and still induce the downstream effects associated with GDF15 and/or TGFβ.

TABLE 2
Peptides used in Example 3 to map the epitope of
the AbCam anti-CLPTM1mAb binding to CLPTM1
(EPR8800 RabMab®). All amino acid residue numbers
are relative to SEQ ID NO: 1.

| Peptide | | Amino acid | SEQ |
|---|---|---|---|
| name | Peptide sequence | residues | ID NO: |
| A5 | SGQVTSNGSIGRDPP | 22-36 | 5 |
| A6 | TSNGSIGRDPPAETQ | 26-40 | 6 |
| A7 | SIGRDPPAETQPQNP | 30-44 | 7 |
| A8 | DPPAETQPQNPPAQP | 34-48 | 8 |
| A9 | ETQPQNPPAQPAPNA | 38-52 | 9 |

### Example 4—Formation of the CLPTM1-TMEM173/STING Complex

### Materials and Methods

Cell Culture

Cells were cultured in 8-well plates, and contacted with GDF15(AbCam) at 2 μg/ml for 1 hr, or a negative control. Medium was aspirated and the cells were rinsed in PBS before fixed in ice-cold PBS supplemented with 3% Formaldehyde for 20 min on ice. Following fixing, the cells were washed 3 times with PBS, permeabilised with 0.5% Triton x-100 in PBS for 15 min RT and blocked o/n with Duolink blocking solution.

Proximity Ligation Assays

Cells were contacted with primary antibodies (rabbit anti-human CLPTM1 Bioss (bs-8018R) and mouse anti-human TMEM173(MAB7169), Santa Cruz) and incubated overnight at 1 μg/ml and washed extensively in PBS-T. Cells were contacted with a pair of secondary antibodies derived from different species (rabbit and mouse) diluted 1:15 for 1 hour at room temperature, and washed extensively and further developed according to manufacturer's instructions for Duolink (Olink Bioscience, Uppsala, Sweden).

Cells were stained with DAPI(blue) for cell nucleus, and PLA-RCA products were detected using detection oligonucleotides labelled with CY3.5(red). Images were acquired as Z-stacks with a Zeiss epiflouroscent microscope and axiovision software.

### Results

Contacting cells with GDF15 resulted in a large increase in the amount of the CLPTM1-TMEM173/STING complex (FIG. 4B) compared with the negative control (FIG. 4A). This indicates that GDF15 can induce CLPTM1 internalisation, and the sequestration of TMEM173/STING.

### Example 5—Degradation of TMEM173/STING and PTP1B Induced by GDF15 and TFG-β3

### Materials and Methods

Cell Culture and Western Blot

NK92 and MCF7 cells were treated with either control (no stimuli), 2 μg/ml GDF15 or 10 ng/ml TGFB3 overnight.

NK92 cells were pelleted by centrifugation and culture media was removed. Cells were subsequently lysed in PBS supplemented with 1% Triton x-100 and Roche complete protease inhibitor cocktail on ice for 20 min.

MCF7 was cultured in reduced serum (0.5% FBS) o/n prior to ligand stimulation. Medium was aspirated from MCF7 cells and cells were lysed with 1% Triton in PBS supplemented with Roche complete protease inhibitor cocktail in wells on ice and collected by cell scraper before transfer to Eppendorf tubes. Lysed cells were cleared by centrifugation 13000 g 10 min at 4° C., denatured in LDS sample buffer at 95° C. for 5 min, and run on 4-12% Gradient gel in MES running buffer, run at low voltage (100 v).

Transfer to a PVDF membrane was performed using TURBO Tank blot system (Biorad). Following transfer, the membrane was blocked in PBS supplemented with 3% BSA, and incubated with the primary antibody overnight. TMEM173/STING and PTP1B primary antibodies from R&D systems was used at 1 μg/ml in PBS. Mouse anti-human TMEM173/STING (R&D systems (MAB7169)) was used to detect TMEM173/STING. Mouse anti-human PTP1B antibodies (AF1366 and AF13661 (R&D systems) were used to detect PTP1B. Secondary HRP-labelled anti-mouse antibodies (Santa Cruz (sc-2005)) diluted 1:2000.

Following Western Blotting, membranes stripped after development using Glycine-SDS buffer, and re-blocked in 3% BSA. Membranes were re-probed using a-GAPDH 1:5000(Sigma) to establish a loading control.

siRNA

For siRNA experiment a 100 μM siRNA (Invitrogen-Life tech) targeting CLPTM1 was transfected into cells using Lipofectamine RNAiMAX and reverse transfection method in MCF7 cells.

### Results

TMEM173/STING Degradation

Contacting cells with GDF15 results in degradation of TMEM173/STING (FIG. 5A, compare lanes 2 and 4). CLPTM1 siRNA down-regulates expression of CLPTM1 and prevents degradation of TMEM173/STING (FIG. 5A, compare lanes 1 and 2). This indicates that GDF15 effects TMEM173/STING degradation via CLPTM1, and that GDF15 results in degradation of TMEM173/STING.

A similar experiment using TGF-β3 and GDF15 indicated that both of these natural ligands for CLPTM1 are able to induce degradation of TMEM173/STING. TMEM173/STING levels were measured in whole cell lysates from NK92 cells (FIG. 5B(i)) and MCF7 cells (FIG. 5B(ii) which had been exposed to TGF-β3 (lane 1), GDF15 (lane 2) or a negative control (lane 3) by Western Blot. Both natural ligands induced degradation of TMEM173/STING (compare lanes 1 and 2 with lane 3).

After detection, the membrane was stripped and GAPDH was measured as a loading control to ensure that similar levels of cell lysates were loaded into each lane (FIG. 5C (i) and (ii)). This indicated that the changes in the levels of TMEM173/STING that were detected were not due to variations in sample loading.

PTP1B Degradation

TGF-β3 and GDF15 were also found to induce PTP1B degradation. PTP1B levels were measured in whole cell lysates from MCF7 cells (FIG. 6A) which had been exposed to TGF-β3 (lane 1), GDF15 (lane 2) or a negative control (lane 3) by Western Blot. Both natural ligands induced degradation of PTP1B (FIG. 6A compare lanes 1 and 2 with lane 3).

After detection, the membrane was stripped and GAPDH was measured as a loading control to ensure that similar levels of cell lysates were loaded into each lane (FIG. 6B). This indicated that the changes in the levels of TMEM173/STING that were detected were not due to variations in sample loading.

### Example 6—Degradation of TMEM173/STING Induced by Anti-CLPTM1 Antibodies

### Materials and Methods

Cell Culture and Western Blot

PBMC was obtained from healthy donor (Buffy coat) and CD14-positive cells were isolated by positive selection using CD14 capture beads (Stem cell technologies). CD14+ Cells were seeded in Costar 24-well ultra-low attachment plates (Corning) and treated with either control or LPS 1 ng/ml o/n. CD14+ cells were stimulated with either ligand, antibody, or ligand and antibodies or antibodies in individual wells overnight. (Abcam (EPR8800 RabMab®) 2 μg/ml. GDF15 2 μg/ml. 3A10 10 μg/ml).

Wells 2, 4, 6 and 7 were stimulated with LPS to increase CLPTM1 expression at the cell surface. Wells 3 and 4 were contacted with the AbCam anti-CLPTM1 antibody. Wells 5-7 were contacted with GDF15 (2 μg/ml). Well 7 was contacted with a mouse anti-CLPTM1 antibody (3A10). After overnight incubation, cells were harvested and whole cell lysates were collected.

Western Blot was performed as in Example 5, but using goat-hTMEM173 RD systems (AF6516) at 1 μg/ml overnight under mild agitation at 4° C. Secondary antibody rabbit anti-goat HRP (Abcam, ab7105) 1:10000 45 min at room temperature. Detection was performed using SuperSignal™ West Dura Extended Duration Substrate (ThermoFisher #34075).

### Results

Stimulation of the cells with LPS resulted in increased expression of CLPTM1, as shown in lanes 1 and 2. Levels of TMEM173/STING were reduced in lanes 4, 6 and 7, compared to the negative control sample in lane 2, indicating that contacting cells with either the AbCam anti-CLPTM1 antibody or GDF15 resulted in degradation of TMEM173/STING. Notably, the lowest level of TMEM173/STING was detected in lane 7, and in particular the level of TMEM173/STING detected in this lane was substantially lower than in lane 6, representing cells contacted by GDF15 only. This indicates that the 3A10 antibody may be able to act synergistically with GDF15.

### Example 7—Incorporation of CLPTM1 into Rab11+ Endosomes Induced by CLPTM1 Agonists

### Materials and Methods

Cell culture was performed as described above in Example 4, but cells were contacted with GDF15, TGF-β3 or the AbCam anti-CLPTM1 antibody (EPR8800 RabMab®). In situ PLA/RCA was performed as described in Example 4, but using mouse anti-human CLPTM1 ((G-7)(sc-374619)) and goat anti-human RAB11. Secondary antibodies were mouse and goat.

### Results

Both GDF15 and TGF-β3 induced formation of Rab11+ endosomes containing CLPTM1 (FIGS. 8B and 8C compared to FIG. 8A). The AbCam anti-CLPTM1 antibody was also found to induce endosome formation to a greater extent than the negative control (FIG. 8D).

### Example 8—Alteration of the Secretome by CLPTM1 Binding Agents

### Materials and Methods

Proseek

Secreted proteins from cell cultures where quantified using multiplexed proximity extension assay, Proseek (Assarsson E et al PlosOne 2014) by Olink Proteomics AB (Uppsala Sweden). Data is presented as Normalized Protein Expression (NPX log 2-scale). The Oncology I v2 92-plex panel was run.

Cell Culture and Cell Stimulation Experiments

A cell suspension of heparinized peripheral blood was diluted 1:1 and placed on a Ficoll Paque Plus. Cells were centrifuged at 400×g for 30 minutes and the PMBC layer was transferred to a 50 mL tube of PBS and centrifuged for 7 min at 250×G.

CD14+ cells were isolated using EasySep Positive Selection kit CD14 (Stemcell inc) according to manufacturer's instructions.

Cell culture was performed in 48 well cell culture plates where seeded with cell suspension at a 0.833×10\hat{ }6 cells/mL in RPMI medium and 300 μL added to each well. For CD14+ cells these where allowed to adhere for 90 minutes at 37 C in an incubator and non-adherent cells where removed.

NK-92 cells were cultured in RPMI-1640 supplemented with 10% FBS, 12.5% Horse serum, L-glut, IL2, 3-mercaptoethanol.

MCF7 cells were cultured in serum free conditions (No FBS added in DMEM) overnight prior to ligand stimulation, and media was replaced 3 times before stimulation to avoid exosomes from FBS. Cells were either treated with control (PBS) or biological active GDF15(Abcam) at 1.5 μg/ml for 1 hr at 37 C.

For analyses of exosomes in FIG. 10A, conditioned media was pre-cleared by centrifugation at 14000 g for 10 min at 4 C, followed by filtration with 0.45 μM filter to avoid contaminants from cell debris, followed by ultracentrifugation at 100000 g for 2 hrs. Pelleted exosome fraction was lysed with 1% Triton X-100 at 4° C., samples diluted in order to have 0.3% Triton concentration and analysed by Proseek multiplex panel as described above.

### Results

The expression of a number of anti-inflammatory cytokines and other secreted proteins was found to be elevated following exposure to TGF-β3, GDF15 or the AbCam anti-CLPTM1 antibody. Midkine (MK) expression was increased by both GDF15 and the AbCam antibody (FIG. 10). GDF15, PIGF, Trail-r2, LAP-TGF-β1, MIC-A, and HB-EGF were all increased by the AbCam anti-CLPTM1 antibody in NK-92 culture, and HB-EGF was also increased by GDF15 in PBMC culture and by the AbCam antibody in MCF7 culture. Caspase-3, MIC-A and TNF-R1 were also increased by the AbCam antibody in MCF7 culture (FIG. 11).

By contrast, the expression of a number of pro-inflammatory cytokines was decreased following exposure to TGF-β3, GDF15 or the AbCam anti-CLPTM1 antibody (FIG. 9). MMP-12 was reduced in PBMC culture by the AbCam anti-CLPTM1 antibody. CXCL9, CXCL11 and IFNγ expression were reduced by TGFβ3 or GDF15 in PBMC culture (see FIG. 11).

### Example 9—Effect of Antibodies on TNFα Response to LPS

### Materials and Methods

Cell Culture

CD14+ cells where isolated and grown in the presence of GDF15 0.5 μg/ml for 24 hours with and without 20 μg/ml of anti-CLPTM1 antibodies 3A10 or 2D12. Following overnight incubation, the cells were stimulated with 1 ng/ml LPS for 4 hours. Culture supernatants where collected and analysed by TNFα ELISA (Mabtech, Stockholm Sweden).

3A10 & 2D12 Antibodies

Mouse monoclonal antibodies 3A10 and 2D12 were raised against CLPTM1 synthetic peptides PWNFLGDELYEQSDE (SEQ ID NO:40) (3A10) and DEEQDSVKVALLET (SEQ ID NO:50) (2D12) using Rapid-Prime™ method by ImmunoPrecise (Victoria, Canada).

### Results

Stimulation of CD14+ cells with LPS resulted in an increase in the amount of TNFα detectable in the conditioned medium. Contacting the cells with either mouse antibody before the cells were stimulated with LPS resulted in a small decrease in the level of TNFα. Contacting the cells with GDF15 alone resulted in a reduction in the level of TNFα. However, contacting the cells with GDF15 and either of the mouse antibodies before the cells were stimulated with LPS led to a greater reduction in the level of TNFα than either the antibodies or GDF15 alone. This suggests that these antibodies may be able to act synergistically with GDF15 to enhance the downstream effects of GDF15.

### Example 10—In Vivo Effects of Anti-CLPTM1 Binding Agent

Mouse monoclonal antibody 7G12 was raised against a CLTPM1 synthetic peptide PWNFLGDELYEQSDE (SEQ ID NO:40) using Rapid-Prime™ method by ImmunoPrecise (Victoria, Canada).

Antibody 59D04 is a fully human phage display derived antibody developed at Yumab (Germany) using a peptide target portion of CLPTM1 (LWRWQLYAAQSTKSPWNFLGDELYEQSDEEQDSVKVALLETNP) (SEQ ID NO:43) and screening on a naive antibody library.

In vivo data generated using 59D04 was using a scFv-mouse IgG1 Fc version produced and purified by Yumab in CHO cells.

### In Vivo Colitis (DSS)

A colitis study was performed by Hooke labs (US). 12 mice where used in each group and dextran sodium sulfate (DSS) induced colitis model in these C57BL/6 mice, 4% DSS in drinking water for 5 days. The study lasted 11 days, from the day of group assignment (Day 0) to the end of the study on Day 10. 200 ug of 7G12 antibody was given i.p. every three days on day 1, 3, and 7 of the study.

Body weight, stool score was measured during the course of the study and the results are shown in Table 3. The administration of 7G12 gave in some measurements a nearly significant improvement over isotype antibody control. Especially in the combined End of study Disease Activity Index (DAI) being 0.1085 (Wilcoxon non-parametric test). Disease activity index (DAI) was calculated by adding the scores for body weight, stool and blood in stool and then dividing by 3. End body weight of the study was also improved with near statistical significance (p-value 0.0872).

At the end of the study, TNF and IL-6 measurements were made in serum and colon samples, and data are shown in Table 4. Statistical tests for the PBS vehicle are performed relative to no DSS, and statistical tests for 7G12 are performed relative to the PBS vehicle. This shows reductions in TNF and IL6 by 7G12 antibody that are nearly significant (2-tailed Student-t test, unequal variance). In comparison, an alternative statistical test, a one-tailed Mann-Whitney test, showed a p-value of 0.0268 of the TNF reduction in serum by 7G12 compared to Vehicle control.

This data taken together illustrate the usefulness of antibodies to CLPTM1 in reducing inflammation in vivo. An antibody with greater affinity, better pharmaco-kinetic properties, etc. is likely to exert greater potency in these in vivo models.

TABLE 3

| | End % body | | End stool | | End DAI | | End colon weight/length | |
|---|---|---|---|---|---|---|---|---|
| Treatment | weight +/− SD | p-value | score +/− SD | p-value | score +/− SD | p-value | ratio +/− SD | p-value |
| DSS/Isotype | 89.2% +/− 9.3% | | 2.4 +/− 1.0 | | 4.8 +/− 2.0 | | 4.2 +/− 0.5 | |
| control | | | | | | | | |
| DSS/7G12 | 94.8% +/− 5.4% | 0.0872 | 2.1 +/− 1.2 | 0.4156 | 3.6 +/− 1.8 | 0.1085 | 4.3 +/− 0.5 | 0.6132 |

TABLE 4

| | TNF, serum | | IL-6, serum | | TNF, colon | | IL-6, colon | |
|---|---|---|---|---|---|---|---|---|
| Treatment | (pg/mL) +/− SD | p-value | (pg/mL) +/− SD | p-value | (pg/mL) +/− SD | p-value | (pg/mL) +/− SD | p-value |
| PBS | 14.9 +/− 20.7 | 0.0419 | 47.9 +/− 94.5 | 0.0935 | 14.3 +/− 14.2 | 0.0174 | 45.2 +/− 75.9 | 0.0619 |
| Vehicle | | | | | | | | |
| 7G12 | 7.3 +/− 3.6 | 0.2197 | 23.0 +/− 11.3 | 0.3737 | 12.3 +/− 4.1 | 0.6400 | 17.1 +/− 13.8 | 0.2198 |

In Vivo Glucose Tolerance (ob/ob)

As CLPTM1 is linked to PTP1B, a master regulator of glucose tolerance, an antibody binding to CLPTM1 was investigated to see if glucose tolerance is improved in obese mice.

Ten ob/ob mice where dosed 4 times twice weekly s.q. at Crownbio (US) with 200 ug of 7G12 or PBS Vehicle for 13 days until oral glucose tolerance test (OGTT) was performed.

Animals were fasted at 06.00 hours, 0 time glucose measured at noon, then dosed with glucose (2 g/kg) to measure glucose tolerance. Serum glucose concentration was measured every 30 minutes, and the results are shown in Table 5 and FIG. 13. A near significant increase in glucose uptake was seen in the animals dosed with the 7G12 antibody. An improved antibody and/or administration at a higher dose are expected to improve the outcome of such a study and provide further evidence of usefulness in obesity/diabetes.

TABLE 5

| | 0 time | 30 min | 60 min | 90 min | 120 min |
|---|---|---|---|---|---|
| | Stat Strip Glu (mg/dl) | | | | |
| PBS Vehicle Mean | 339.80 | 674.30 | 524.60 | 440.80 | 461.40 |
| St. Dev. | 150.55 | 111.89 | 134.30 | 148.81 | 173.06 |
| 7G12 Mean | 274.80 | 631.90 | 472.80 | 383.60 | 366.90 |
| St. Dev. | 66.80 | 37.26 | 69.06 | 66.92 | 77.03 |
| p-value (T-test, | 0.23 | 0.27 | 0.29 | 0.28 | 0.13 |
| 2-tailed, equal variance) | | | | | |
| p-value (Mann-Whitney | 0.248 | 0.144 | 0.192 | 0.121 | 0.060 |
| (1 sided) | | | | | |

### In Vivo Arthritis (CAIA)

A Collagen Antibody Induced Arthritis (CAIA) study was performed to evaluate the effect of 7G12 on disease activity. Experiments performed by Redoxis AB (Lund, Sweden). DBA/1 male mice where given 2 mg monoclonal anti-CII antibodies (MD Bioproducts) on day 0. Day 3 was boosted with i.p. 50 ug LPS per mouse. 7G12 was given i.v. from day 1 and then every other day 200 ug per mouse and dose to 20 days. Enbrel, a TNFα blocker (Pfizer), was used as a positive control at 5 mg/kg i.p. dose and assayed for potential synergy with 7G12. Eight mice where in each group. Disease was macroscopically evaluated daily in a blinded fashion using a standard scoring system.

Average disease severity score, maximum disease severity score, total disease severity score and inhibition are shown in FIG. 14. These results indicate a possible synergistic effect of TNF blockade by Enbrel combined with 7G12.

In Vivo Sepsis (LPS) #1

5 mice where given i.v. 200 ug of the 7G12 antibody and compared to 5 mice with PBS vehicle control. After 1 hour 100 ng of LPS was injected i.v. After 2 hours blood was drawn and pro-inflammatory cytokine concentrations measured. Experiments and analyses where performed by Hookelabs (US). Nearly significant lower TNF secretion was seen with the CLPTM1 antibody 7G12, two-tailed T-test of unequal variance. A significant lower secretion of IL6 was seen. Measurements of TNF and IL-6 are shown in Table 6

These results indicate a usefulness of CLPTM1 targeting antibodies to dampen inflammation.

In Vivo Sepsis (LPS) #2

An additional in vivo mouse sepsis model test was performed by Redoxis (Lund, Sweden). 20 ng LPS was injected 1 hour after antibody and controls administration by i.v. A one-tailed Mann-Whitney test showed statistically significant immune suppressive properties of 7G12 and 59D04 compared to PBS control. Measurements of TNF and IL-6 are shown in Table 7.

TABLE 6

| | TNF | TNF | IL-6 | IL-6 |
|---|---|---|---|---|
| | average pg/ml | stdev | average pg/ml | stdev |
| Control (PBS) | 511 | 48 | 5655 | 375 |
| 7G12 | 357 | 81 | 3078 | 725 |
| p-value | 0.143 | | 0.013 | |

TABLE 7

| | TNF | TNF | IL-6 | IL-6 |
|---|---|---|---|---|
| | average pg/ml | stdev | average pg/ml | stdev |
| Control (PBS) | 806 | 144 | 73752 | 14278 |
| 7G12 (160 ug) | 541 | 235 | 55734 | 17887 |
| p-value vs PBS | 0.037 | | 0.047 | |
| 59D04 (50 ug) | 681 | 159 | 55408 | 13366 |
| p-value vs PBS | 0.200 | | 0.030 | |

### In Vivo MS (EAE)

Experimental Autoimmune Encephalomyelitis (EAE) is an established model for Multiple Sclerosis in mice. mAb 7G12 was evaluated for efficacy in this model in comparison to IFNb and in combination with IFNb. Recombinant IFNβ is an important drug for treating MS.

7-10 week old C57Bl/6 mice were used in the study. Immunization was initiated with 200 ug MOG35-55 peptide with Incomplete Freund's Adjuvant+ Pertussis toxin (200 ng) on day 0 and 2. Drug was administered i.v. for mAb starting on day 5 and s.c. for mouse IFNβ (PBL) at 10000 U per dose. Work was performed by Redoxis AB (Lund, Sweden).

Disease was evaluated with a blinded scoring system from day 5 using a macroscopic system 0=healthy, 1=tail weakness, 2=tail paralysis, 3=tail paralysis and mild waddle, 4=tail paralysis and severe waddle, 5=tail paralysis and paralysis of one limb, 6=tail paralysis and paralysis of a pair of limbs, 7=tetraparesis or paralysis of three limbs and 8=premorbid or dead.

7G12 mAb was given either as derived from a mouse hybridoma (7G12hyb) at 0.1 mg doses or as a recombinant mAb (7G12rec) at 0.3 mg doses both formulated in sterile PBS. On day 9 the disease severity was significantly lower for 7G12rec at 0.3 mg dose, p-value 0.0496 (Students two tailed T-test with equal variance). Data beyond day 9 did not show significant differences between the groups. Data are shown in FIG. 15.

7G12hyb at 0.1 mg dose and IFNβ treatment in combination was evaluated. On day 7 disease severity was significantly lower with the combination treatment compared to PBS vehicle (p-value 0.004, Students two tailed T-test with equal variance). Disease severity on day 7 was also significantly better with the combination therapy compared to IFNb alone (p-value 0.031, Students two tailed T-test with equal variance). On day 9 the combination treatment also showed improvement over PBS vehicle (p-value 0.014, Students two tailed T-test with equal variance). Data beyond day 9 did not show significant differences between the groups. Data are shown in FIG. 16. The combination treatment show promise in the possibility to delay disease onset.

Variable heavy sequence of 59D04 is
(SEQ ID NO: 41)
EVQLVESGGGVVQPGRSLRLSCAASGFTFSTYAMHWVRQAPGKGLEWVAV
ISYDGTNKYYADSVKGRFTIFRDNSKNTLYLQMNSLRAEDTAVYYCGSGS
YWGQGTLVTVSS
Variable light sequence of 59D04 is
(SEQ ID NO: 42)
QPVLTQPASVSGSPGQSITISCTGTSSDVGGYNYVSWYQQHPGKAPKLMI
YEVTNRPSGVSDRFSGSKSGNTASLTISGLQAEDEADYYCSSYKSSNTVV
FGGGTKVTVL
Variable heavy sequence of 7G12 (IgG1 isotype) is
(SEQ ID NO: 44)
QVQLQQSGTELVRPGTSVKVSCKASGYAFTNYLIEWVKQRPGQGLEWIGV
INPGSGGTRYNEKFKGKATLTADKSSTTAHMQLSSLTSDDSAVYCARWGG
NYSGYAMDYWGQGTSVTVSS
Variable light sequence of 7G12 (Kappa isotype) is
(SEQ ID NO: 45)
QIVLTQSPVIMSASPGEKVTITCSASSSVSYMHWFQQKPGTSPKLWIYST
SNLASGVPARFSGSGSGTSYSLTISRMEAEDAATYYCQQRSSYPPTFGAG
TKL.

Consensus DNA sequences encoding the heavy and light chains for 7G12 are provided as SEQ ID NOs:46 and 47, respectively.

### Example 11—In Vitro Testing of Human Antibodies

A series of fully human naive phage display library derived monoclonal antibodies (Yumab, Germany) selected for CLPTM1 binding were tested for immune suppressive ability on human CD14+ cells stimulated in vitro with LPS and cytokine secretion measured. Antibodies where tested as scFv-mouse IgG1-Fc at 1 ug/ml of antibody was incubated with the cells overnight and LPS 1 ng/ml added and supernatants collected after 4 hours. Supernatants were assayed with Olink Proteomics AB multiplex Oncology v2 panel. TNF and IL12 measurements are shown in FIG. 17 illustrating immunosuppressive activity. Averages of triplicate measurements are shown in FIG. 17.

Antibodies were phage display selected against synthetic peptide portions of CLPTM1.

(SEQ ID NO: 43)
59: LWRWQLYAAQSTKSPWNFLGDELYEQSDEEQDSVKVALLETNP
(SEQ ID NO: 48)
60: DTLMNLHVYISEHEHFTDFNATSALFWEQHDLVYGDWTSG
(SEQ ID NO: 49)
61: SGDYYPIIYFNDYWNLQKDYYPINESLA

### Example 12—Antibody Affinity Determination

Affinity of antibodies was determined by finding the half-maximal binding on the native target in a flow cytometry assay. Since the native target is found inside most cells and rarely on the cell surface, the assay was performed using plasma membrane-permeabilized cells, an intra-cellular staining protocol. Using the native target is more applicable when evaluating and comparing various antibodies as it is the true antigen to be bound in a therapeutic setting in vivo. 0-876 cells were permeabilized with BD-fix and permeabilization buffer (554722 BD-Biosciences). Antibody at various concentrations was incubated with cells for 30 minutes at +4 C, then washed twice with wash buffer (BD-Biosciences 554723). Secondary antibodies a-mouse IgG-PE (Molecular Probes) was used for mouse monoclonal antibodies according to manufacturer's instructions and a-rabbit-IgG-PE (Molecular Probes) for rabbit monoclonal antibody (Abcam ERP8800). Three washes followed and fluorescence intensity in cells was quantified by FACS Cantoll in PBS with cells in 2% FCS buffer.

7G12 had the greatest affinity showing more than having half maximal binding (Flow Signal) at 0.1 μg/mL suggesting a Kd (as defined by half maximal binding) below 1 nM. The assay was performed on 0-876 cancer cell line. Binding data are shown in FIG. 18. Antibodies 7G12, 59D04 and the Abcam antibody showed more half maximal binding at 1 μg/mL.

The Abcam antibody against CLPTM1 has reported affinity according to Biacore data supplied by the manufacturer of 0.0345 nM Kd. This was derived on a non-native recombinant portion of the antigen as test.

Of these antibodies 7G12 and 59D04 showed in vivo immune suppressive properties in an a general immune activation model using LPS. The Abcam and Santa Cruz mAbs have not been tested in vivo but the Abcam antibody has shown immune suppression activity in other tests, while Santa Cruz (G-7) has been inconclusive.

### Example 13—Down-Regulation of STING Expression by CLPTM1 Antibodies

Down-regulation of STING protein is desirable in several autoimmune diseases, most notably interferonopathies such as SLE. Using an immune cell based assay we tested the ability of a series of antibodies to down regulate STING as measured by Flow Cytometry.

PBMCs derived from buffy coats from healthy blood donors were cultured in cRPMI (10% FCS, 1% Glutamine) in the presence of 1 ng/mL LPS, with and without 10 ug/mL antibody (59D04 and 7G12 respectively). After 18 h, cells were harvested and stained for CD14 and STING expression with BD Cytofix/Cytoperm™ and BD Perm/Wash™ buffer (BD Biosciences) according to the manufacturer's instructions. Cell viability was assessed with LIVE/DEAD™ Violet Viability/Vitality Kit (Life Technologies). Data was collected on a BD FACSCanto flow cytometer and analyzed using Flowjo vX software.

Data was evaluated from lymphocytes showing down-regulation of STING protein by 59D04 and 7G12 as seen in the number of STING positive cells.

When the analysis was gated for CD14+ monocytes a reduction in signal intensity was seen in the Mean Fluorescence Intensity (MFI) by mAbs 59D04 and 7G12. Standard deviation in the figure below illustrate the variability of intensity among single cells.

Data for total lymphocytes and CD14+ monocytes are shown in FIG. 19.

### Example 14—Low Affinity Monoclonal Antibody (Santa Cruz G7) is not Capable of Immunosuppression

PBMC were obtained from a healthy donor (Buffy coat). 20,000 cells per well were seeded and grown in 200 uL complete RPMI 1640 medium (10% FBS, 1% Glut, 1% PeSt) with Abcam 5 ug/mL and 10 ug/mL for Santa Cruz and IgG2a isotype control over night. 1 ng/mL LPS was added and supernatant was analyzed for secreted proteins after 4 hours.

Secreted proteins from cell cultures were quantified using a multiplexed proximity extension assay, Proseek by Olink Proteomics AB (Uppsala Sweden). NPX values (log 2) from Proseek data were transformed to linear scale normalized to Isotype control (100%). The results are shown in FIG. 20, from which it can be seen that IFNγ showed lower secretion in the cells treated with the Abcam antibody but not the Santa Cruz antibody illustrating the need for affinity to enable immune suppression by an anti-CLPTM1 antibody.

1. A binding agent capable of binding to the extracellular domain of CLPTM1 for use in the treatment or prevention of a condition which is responsive to, or benefits from, (i) immunosuppression, (ii) the reduction or reversal of one or more pro-inflammatory cytokines or the induction of an anti-inflammatory cytokine), (iii) an increase in insulin sensitivity, or (iv) therapy with GDF15 and/or TGF-β and/or IFNβ, wherein said binding agent has an EC₅₀ value of 1 μg/ml or less when determined by measuring binding to membrane-permeabilised 0-876 cells expressing native CLPTM1 by flow cytometry, and wherein said binding agent is not a natural ligand for CLPTM1.

2. A binding agent for use according to claim 2, wherein said binding agent is for use as an immunosuppressive agent, an anti-inflammatory agent, an anti-insulin resistance agent and/or a cardioprotective agent.

3. A binding agent according to claim 1 or 2, wherein the binding agent when pre-incubated at 1 ug/ml for 16 hours with CD14+ cells induces (i) a 3-fold decrease in the level of secreted TNFα when the CD14+ cells are stimulated with 1 ng/ml LPS for 4 hours, and/or (ii) a 2-fold decrease in the level of secreted IL12 when the CD14+ cells are stimulated with 1 ng/ml LPS for 4 hours.

4. The binding agent for use according to any one of claims 1 to 3, wherein the binding agent is capable of down-regulating TMEM173/STING and/or PTP1B.

5. The binding agent for use according to claim 4, wherein the binding agent is capable of down-regulating TMEM173/STING.

6. The binding agent for use of any one of claims 1 to 4, wherein the binding agent is not GDF15 and/or TGF-β.

7. The binding agent for use of any one of claims 1 to 5, wherein the binding agent is an antibody.

8. The binding agent for use of claim 6, wherein the antibody is a polyclonal or monoclonal antibody or a fragment thereof.

9. The binding agent for use of claim 6 or 7, wherein the antibody is a chimeric or humanised antibody, or a human antibody.

10. The binding agent for use of any one of claims 1 to 8, wherein said binding agent binds to a polypeptide having or comprising an amino acid sequence as set forth in any one of SEQ ID NOs:3-9, 40 or 43.

11. The binding agent for use according to any one of claims 1 to 10 for use in immunosuppression or in the treatment or prevention of an inflammatory condition, a metabolic disorder, including a metabolic disorder associated with insulin resistance, or a condition involving damage to the heart.

12. The binding agent for use according to any one of claims 1 to 11, for use in the treatment or prevention of an autoimmune condition.

13. The binding agent for use according to any one of claims 1 to 12, wherein the condition is multiple sclerosis (MS), Systemic Lupus Erythematosus (SLE), juvenile arthritis or rheumatoid arthritis.

14. The binding agent for use according to any one of claims 1 to 12, wherein the condition is a condition of the GI tract, including Crohn's disease, ulcerative colitis or other inflammatory bowel disease, or wherein the condition is coronary artery disease or atherosclerosis.

15. The binding agent for use according to any one of claims 1 to 11, wherein the condition is a haematological disorder or haematopoietic cancer.

16. The binding agent for use according to claim 15, wherein the condition is leukemia or lymphoma.

17. The binding agent for use according to claim 15, wherein the condition is Waldenström macroglobulinemia.

18. The binding agent for use according to any one of claims 1 to 11, wherein the condition is an infectious disease or an infection with an intracellular pathogen.

19. The binding agent for use according to claim 18, wherein the infectious disease or infection is tuberculosis or malaria.

20. The binding agent for use according to any one of claims 1 to 11, for use in treating or preventing organ or tissue rejection following transplant.

21. The binding agent for use according to any one of claims 1 to 11, for use in the treatment or prevention of local internal inflammation, scars, fibrosis or radiation-induced damage.

22. The binding agent for use according to any one of claims 1 to 11, wherein the condition is an allergy or allergic reaction.

23. The binding agent for use according to any one of claims 1 to 11, wherein the condition is STING-associated vasculopathy with onset in infancy (SAVI).

24. The binding agent for use according to any one of claims 1 to 11, wherein the condition is non-alcoholic fatty liver disease (NAFLD), preferably wherein the condition is NASH.

25. The binding agent for use according to any one of claims 1 to 11, wherein the condition is preeclampsia.

26. The binding agent for use according to any one of claims 1 to 11, wherein the condition is a lung disorder.

27. The binding agent for use according to claim 26, wherein the condition is emphysema or COPD.

28. The binding agent for use according to any one of claims 1 to 11, for use in the treatment or prevention of insulin resistance or a condition associated therewith.

29. The binding agent for use according to claim 28, wherein the condition is type 2 diabetes or metabolic syndrome.

30. The binding agent for use according to any one of claims 1 to 11 for use as a cardioprotective agent.

31. The binding agent for use according to claim 30, for use in treating, reducing or preventing myocardial damage arising from acute myocardial infarction (AMI) or other acute coronary syndrome or ischaemic or hypoxic condition.

32. A method of treating or preventing a condition which is responsive to, or benefits from, (i) immunosuppression, (ii) the reduction or reversal of one or more pro-inflammatory cytokines or the induction of an anti-inflammatory cytokine, (iii) an increase in insulin sensitivity, or (iv) therapy with GDF15 and/or TGF-β3, said method comprising administering to a subject in need thereof an effective amount of a binding agent as defined in any one of claims 1 to 10.

33. Use of a binding agent as defined in any one of claims 1 to 10 in the manufacture of a medicament for use in the treatment or prevention of a condition which is responsive to, or benefits from, (i) immunosuppression, (ii) the reduction or reversal of one or more pro-inflammatory cytokines or the induction of an anti-inflammatory cytokine, (iii) an increase in insulin sensitivity, or (iv) therapy with GDF15 and/or TGF-β3.

34. A binding agent capable of binding to the extracellular domain of CLPTM1, for use in the treatment or prevention of NASH, preeclampsia, haematological disorders, including haematological cancers, lung disorders, including emphysema or COPD, an infection with an intracellular pathogen, nephritis, transplant rejection, GvHD, MODS, MOF, MOFS, inflammatory bowel disease, including Crohn's disease or ulcerative colitis, coronary heart disease or atherosclerosis, or as a cardioprotective agent, wherein said binding agent has an EC₅₀ value of 1 μg/ml or less when determined by measuring binding to membrane-permeabilised 0-876 cells expressing native CLPTM1 by flow cytometry.

35. A binding agent capable of binding to CLPTM1 for use in the treatment or prevention of a metabolic disorder, including insulin resistance or a condition associated therewith.

36. A product comprising a binding agent capable of binding to CLPTM1 and interferon-β as a combined preparation for separate, simultaneous or sequential use in the treatment of an autoimmune or inflammatory condition, including multiple sclerosis.

37. A product comprising a binding agent capable of binding to CLPTM1 and a TNF-blocker as a combined preparation for separate, simultaneous or sequential use in the treatment of an autoimmune or inflammatory condition.

38. A binding agent which binds human CLPTM1 and which comprises the complementarity-determining regions (CDRs) VLCDR1, VLCDR2, VLCDR3, VHCDR1, VHCDR2 and VHCDR3, wherein
(i) each of said CDRs has an amino acid sequence as follows:
VLCDR1 has the sequence set forth in SEQ ID NO: 51;
VLCDR2 has the sequence set forth in SEQ ID NO: 52;
VLCDR3 has the sequence set forth in SEQ ID NO: 53;
VHCDR1 has the sequence set forth in SEQ ID NO: 54;
VHCDR2 has the sequence set forth in SEQ ID NO: 55; and
VHCDR3 has the sequence set forth in SEQ ID NO: 56;
or, for each sequence, an amino acid sequence with at least 85% sequence identity thereto,
or wherein one or more of said CDR sequences of SEQ ID NOs: 51 to 56 may optionally be modified by substitution, addition or deletion of 1 to 3 amino acids; or
(ii) each of said CDRs has an amino acid sequence as follows:
VLCDR1 has the sequence set forth in SEQ ID NO: 57;
VLCDR2 has the sequence set forth in SEQ ID NO: 58;
VLCDR3 has the sequence set forth in SEQ ID NO: 59;
VHCDR1 has the sequence set forth in SEQ ID NO: 60;
VHCDR2 has the sequence set forth in SEQ ID NO: 61; and
VHCDR3 has the sequence set forth in SEQ ID NO: 62;
or, for each sequence, an amino acid sequence with at least 85% sequence identity thereto,
or wherein one or more of said CDR sequences of SEQ ID NOs: 57 to 62 may optionally be modified by substitution, addition or deletion of 1 to 3 amino acids.

39. The binding agent of claim 38 wherein said binding agent comprises
(i) a VL region having an amino acid sequence as set forth in SEQ ID NO: 45, or an amino acid sequence having at least 80% sequence identity thereto, and a VH region having an amino acid sequence as set forth in SEQ ID NO: 44, or an amino acid sequence having at least 80% sequence identity thereto; or
(ii) a VL region having an amino acid sequence as set forth in SEQ ID NO: 42, or an amino acid sequence having at least 80% sequence identity thereto, and a VH region having an amino acid sequence as set forth in SEQ ID NO: 41, or an amino acid sequence having at least 80% sequence identity thereto.

40. The binding agent of claim 38 or claim 39 wherein said binding agent is an antibody.


SEQUENCE LISTING (62 sequences)

SEQ ID NO: 1 - protein, 669 aa, Homo sapiens
MAAAQEADGARSAVVAAGGGSSGQVTSNGSIGRDPPAETQPQNPPAQPAPNAWQVIKGVLFRIFIIWAISSWFRRGPAPQDQAGPGGAPRVASRNLFPKDTLMNLHVYISEHEHFTDFNATSALFWEQHDLVYGDWTSGENSDGCYEHFAELDIPQSVQQNGSIYIHVYFTKSGFHPDPRQKALYRRLATVHMSRMINKYKRRRFQKTKNLLTGETEADPEMIKRAEDYGPVEVISHWHPNITINIVDDHTPWVKGSVPPPLDQYVKFDAVSGDYYPIIYFNDYWNLQKDYYPINESLASLPLRVSFCPLSLWRWQLYAAQSTKSPWNFLGDELYEQSDEEQDSVKVALLETNPYLLALTIIVSIVHSVFEFLAFKNDIQFWNSRQSLEGLSVRSVFFGVFQSFVVLLYILDNETNFVVQVSVFIGVLIDLWKITKVMDVRLDREHRVAGIFPRLSFKDKSTYIESSTKVYDDMAFRYLSWILFPLLGCYAVYSLLYLEHKGWYSWVLSMLYGFLLTFGFITMTPQLFINYKLKSVAHLPWRMLTYKALNTFIDDLFAFVIKMPVMYRIGCLRDDVVFFIYLYQRWIYRVDPTRVNEFGMSGEDPTAAAPVAEVPTAAGALTPTPAPTTTTATREEASTSLPTKPTQGASSASEPQEAPPKPAEDKKKD

SEQ ID NO: 2 - protein, 353 aa, Homo sapiens
AAAQEADGARSAVVAAGGGSSGQVTSNGSIGRDPPAETQPQNPPAQPAPNAWQVIKGVLFRIFIIWAISSWFRRGPAPQDQAGPGGAPRVASRNLFPKDTLMNLHVYISEHEHFTDFNATSALFWEQHDLVYGDWTSGENSDGCYEHFAELDIPQSVQQNGSIYIHVYFTKSGFHPDPRQKALYRRLATVHMSRMINKYKRRRFQKTKNLLTGETEADPEMIKRAEDYGPVEVISHWHPNITINIVDDHTPWVKGSVPPPLDQYVKFDAVSGDYYPIIYFNDYWNLQKDYYPINESLASLPLRVSFCPLSLWRWQLYAAQSTKSPWNFLGDELYEQSDEEQDSVKVALLETNP

SEQ ID NO: 3 - protein, 4 aa, Homo sapiens
DPPA

SEQ ID NO: 4 - protein, 9 aa, Homo sapiens
GRDPPAETQ

SEQ ID NO: 5 - protein, 15 aa, Homo sapiens
SGQVTSNGSIGRDPP

SEQ ID NO: 6 - protein, 15 aa, Homo sapiens
TSNGSIGRDPPAETQ

SEQ ID NO: 7 - protein, 15 aa, Homo sapiens
SIGRDPPAETQPQNP

SEQ ID NO: 8 - protein, 15 aa, Homo sapiens
DPPAETQPQNPPAQP

SEQ ID NO: 9 - protein, 15 aa, Homo sapiens
ETQPQNPPAQPAPNA

SEQ ID NO: 10 - protein, 21 aa, Homo sapiens
AAAQEADGARSAVVAAGGGSG

SEQ ID NO: 11 - protein, 21 aa, Homo sapiens
SGQVTSNGSIGRDPPAETQPG

SEQ ID NO: 12 - protein, 21 aa, Homo sapiens
QNPPAQPAPNAWQVIKGVLFG

SEQ ID NO: 13 - protein, 21 aa, Homo sapiens
RIFIIWAISSWFRRGPAPQDG

SEQ ID NO: 14 - protein, 21 aa, Homo sapiens
QAGPGGAPRVASRNLFPKDTG

SEQ ID NO: 15 - protein, 21 aa, Homo sapiens
LMNLHVYISEHEHFTDFNATG

SEQ ID NO: 16 - protein, 21 aa, Homo sapiens
SALFWEQHDLVYGDWTSGENG

SEQ ID NO: 17 - protein, 21 aa, Homo sapiens
SDGCYEHFAELDIPQSVQQNG

SEQ ID NO: 18 - protein, 21 aa, Homo sapiens
GSIYIHVYFTKSGFHPDPRQG

SEQ ID NO: 19 - protein, 21 aa, Homo sapiens
KALYRRLATVHMSRMINKYKG

SEQ ID NO: 20 - protein, 21 aa, Homo sapiens
RRRFQKTKNLLTGETEADPEG

SEQ ID NO: 21 - protein, 21 aa, Homo sapiens
MIKRAEDYGPVEVISHWHPNG

SEQ ID NO: 22 - protein, 21 aa, Homo sapiens
ITINIVDDHTPWVKGSVPPPG

SEQ ID NO: 23 - protein, 21 aa, Homo sapiens
LDQYVKFDAVSGDYYPIIYFG

SEQ ID NO: 24 - protein, 21 aa, Homo sapiens
NDYWNLQKDYYPINESLASLG

SEQ ID NO: 25 - protein, 21 aa, Homo sapiens
PLRVSFCPLSLWRWQLYAAQG

SEQ ID NO: 26 - protein, 21 aa, Homo sapiens
STKSPWNFLGDELYEQSDEEG

SEQ ID NO: 27 - protein, 21 aa, Homo sapiens
YEQSDEEQDSVKVALLETNPG

SEQ ID NO: 28 - protein, 21 aa, Homo sapiens
LWRWQLYAAQSTKSPWNFLGG

SEQ ID NO: 29 - protein, 21 aa, Homo sapiens
YPINESLASLPLRVSFCPLSG

SEQ ID NO: 30 - protein, 21 aa, Homo sapiens
GDYYPIIYFNDYWNLQKDYYG

SEQ ID NO: 31 - protein, 21 aa, Homo sapiens
WQVIKGVLFRIFIIWAISSWG

SEQ ID NO: 32 - protein, 21 aa, Homo sapiens
RNLFPKDTLMNLHVYISEHEG

SEQ ID NO: 33 - protein, 21 aa, Homo sapiens
FTDFNATSALFWEQHDLVYGG

SEQ ID NO: 34 - protein, 20 aa, Homo sapiens
GSIYIHVYFTKSGFHPDPRQ

SEQ ID NO: 35 - protein, 20 aa, Homo sapiens
KALYRRLATVHMSRMINKYK

SEQ ID NO: 36 - protein, 20 aa, Homo sapiens
ITINIVDDHTPWVKGSVPPP

SEQ ID NO: 37 - protein, 20 aa, Homo sapiens
LDQYVKFDAVSGDYYPIIYF

SEQ ID NO: 38 - protein, 20 aa, Homo sapiens
YPINESLASLPLRVSFCPLS

SEQ ID NO: 39 - protein, 20 aa, Homo sapiens
GDYYPIIYFNDYWNLQKDYY

SEQ ID NO: 40 - protein, 15 aa, Homo sapiens
PWNFLGDELYEQSDE

SEQ ID NO: 41 - protein, 112 aa, Homo sapiens
EVQLVESGGGVVQPGRSLRLSCAASGFTFSTYAMHWVRQAPGKGLEWVAVISYDGTNKYYADSVKGRFTIFRDNSKNTLYLQMNSLRAEDTAVYYCGSGSYWGQGTLVTVSS

SEQ ID NO: 42 - protein, 110 aa, Homo sapiens
QPVLTQPASVSGSPGQSITISCTGTSSDVGGYNYVSWYQQHPGKAPKLMIYEVTNRPSGVSDRFSGSKSGNTASLTISGLQAEDEADYYCSSYKSSNTVVFGGGTKVTVL

SEQ ID NO: 43 - protein, 43 aa, Homo sapiens
LWRWQLYAAQSTKSPWNFLGDELYEQSDEEQDSVKVALLETNP

SEQ ID NO: 44 - protein, 120 aa, Mus musculus
QVQLQQSGTELVRPGTSVKVSCKASGYAFTNYLIEWVKQRPGQGLEWIGVINPGSGGTRYNEKFKGKATLTADKSSTTAHMQLSSLTSDDSAVYCARWGGNYSGYAMDYWGQGTSVTVSS

SEQ ID NO: 45 - protein, 103 aa, Mus musculus
QIVLTQSPVIMSASPGEKVTITCSASSSVSYMHWFQQKPGTSPKLWIYSTSNLASGVPARFSGSGSGTSYSLTISRMEAEDAATYYCQQRSSYPPTFGAGTKL

SEQ ID NO: 46 - DNA, 363 bp, Mus musculus
caggtccagctgcagcagtctggaactgaactggtgaggcctgggacttcagtgaaggtgtcctgcaaggcttctggatacgccttcactaattacttgatagagtgggtaaagcagaggcctggacagggccttgagtggattggagtgattaatcctggaagtggtggtactaggtacaatgagaagttcaagggcaaggcaacactgactgcagacaaatcctccaccactgcccacatgcagctcagcagcctgacatctgatgactctgcggtctatttctgtgcaagatgggggggaaactactctggctatgctatggactactggggtcaaggaacctcagtcaccgtctcctca

SEQ ID NO: 47 - DNA, 318 bp, Mus musculus
caaattgttctcacccagtctccagtaatcatgtctgcatctccaggggagaaggtcaccataacctgcagtgccagctcaagtgtaagttacatgcactggttccagcagaagccaggcacttctcccaaactctggatttatagcacatccaacctggcttctggagtccctgctcgcttcagtggcagtggatctgggacctcttactctctcacaatcagccgaatggaggctgaagatgctgccacttattactgccagcaaaggagtagttacccacccacgttcggtgctgggaccaagctggagctgaaa

SEQ ID NO: 48 - protein, 40 aa, Homo sapiens
DTLMNLHVYISEHEHFTDFNATSALFWEQHDLVYGDWTSG

SEQ ID NO: 49 - protein, 28 aa, Homo sapiens
SGDYYPIIYFNDYWNLQKDYYPINESLA

SEQ ID NO: 50 - protein, 14 aa, Homo sapiens
DEEQDSVKVALLET

SEQ ID NO: 51 - protein, 10 aa, Mus musculus
SASSSVSYMH

SEQ ID NO: 52 - protein, 7 aa, Mus musculus
STSNLAS

SEQ ID NO: 53 - protein, 9 aa, Mus musculus
QQRSSYPPT

SEQ ID NO: 54 - protein, 5 aa, Mus musculus
NYLIE

SEQ ID NO: 55 - protein, 17 aa, Mus musculus
VINPGSGGTRYNEKFKG

SEQ ID NO: 56 - protein, 11 aa, Mus musculus
ARWGGNYSGYA

SEQ ID NO: 57 - protein, 14 aa, Homo sapiens
TGTSSDVGGYNYVS

SEQ ID NO: 58 - protein, 8 aa, Homo sapiens
YEVTNRPS

SEQ ID NO: 59 - protein, 10 aa, Homo sapiens
SSYKSSNTVV

SEQ ID NO: 60 - protein, 13 aa, Homo sapiens
AASGFTFSTYAMH

SEQ ID NO: 61 - protein, 10 aa, Homo sapiens
VISYDGTNKY

SEQ ID NO: 62 - protein, 5 aa, Homo sapiens
GSGSY